(19) United States
(12) Patent Application Publication
(10) Pub. No.: US 2026/0335286 A1
(43) Pub. Date: Oct. 29, 2026

(54) COMPOSITIONS AND METHODS FOR TREATING WEIGHT CONDITIONS

(71) Applicant: Corbus Pharmaceuticals, Inc., Norwood, MA (US)

(72) Inventors: Marshall MORNINGSTAR, Framingham, MA (US); Andrew KOLODZIEJ, Winchester, MA (US); Thomas F. PARSONS, Waltham, MA (US); Laura TRESPIDI, Il-Qala (MT); William MOORE, Folkestone (GB)

(21) Appl. No.: 19/629,313

(22) Filed: Mar. 26, 2026

Related U.S. Application Data
(60) Provisional application No. 63/778,185, filed on Mar. 26, 2025.
(60) Provisional application No. 63/938,005, filed on Dec. 10, 2025.
(60) Provisional application No. 63/989,873, filed on Feb. 24, 2026.

(51) Int. Cl. A61K 31/415; A61P 3/04

(52) U.S. Cl. CPC A61K 31/415; A61P 3/04

(57) ABSTRACT

The present invention features compositions of CRB-913 and methods for treating weight conditions, weight related conditions, and co-morbidities of weight conditions with CRB-913 and compositions thereof.

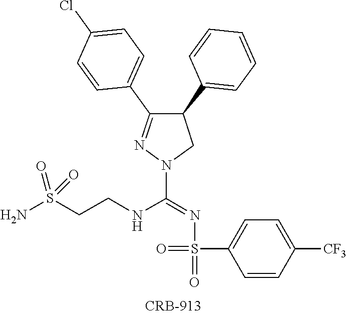 [SMILES: NS(=O)(=O)CCN/C(=N\S(=O)(=O)c1ccc(C(F)(F)F)cc1)N1C[C@H](c2ccccc2)C(c2ccc(Cl)cc2)=N1]

### BACKGROUND

Weight conditions, such as obesity, are associated with numerous dangerous co-morbidities.

Obesity is associated with an increase in the overall amount of adipose tissue (i.e., body fat), especially adipose tissue localized in the abdominal area. Obesity has reached epidemic proportions in the United States. The prevalence of obesity has steadily increased over the years among all racial and ethnic groups. The most recent data from the Centers for Disease Control and Prevention, and the National Center for Health Statistics report 66% of the adult population overweight (BMI, 25.0-29.9 kg/m²), 31% obese (BMI, 30-39.9 kg/m²), and 5% extremely obese (BMI, ≥40.0 kg/m²). Among children aged 6 through 19 years, 32% were overweight and 17% were obese. This translates to 124 million Americans medically overweight, and 44 million of these deemed obese. Obesity is responsible for more than 300,000 deaths annually and will soon overtake tobacco usage as the primary cause of preventable death in the United States. Obesity is a chronic disease that contributes directly to numerous dangerous co-morbidities, including type 2 diabetes, cardiometabolic diseases, hepatic disorders, cardiovascular diseases, inflammatory diseases, sleeping disorders, premature aging, and some forms of cancer. Type 2 diabetes, a serious and life-threatening disorder with growing prevalence in both adult and childhood populations, is currently the 7^{th} leading cause of death in the United States. Since more than 80% of patients with type 2 diabetes are overweight, obesity is the greatest risk factor for developing type 2 diabetes. Increasing clinical evidence indicates that the best way to control type 2 diabetes is to reduce weight. Accordingly, there is a continuing need for the development of improved medications that treat or prevent obesity.

Cannabinoid receptors (CB1 and CB2) and their endogenous ligands (e.g., anandamide, 2-AG) play a prominent role in the control of food intake and energy metabolism. CB1 receptors are widely expressed in the brain, including cortex, hippocampus, amygdala, pituitary and hypothalamus. CB1 receptors have also been identified in numerous peripheral organs and tissues, including thyroid gland, adrenal gland, reproductive organs, adipose tissue, liver, muscle, pancreas, kidney, and gastrointestinal tract. CB2 receptors are localized almost exclusively in immune and blood cells (Endocrine Reviews 2006, 27, 73).

The plant-derived cannabinoid agonist Δ⁹-tetrahydrocannabinol (Δ⁹-THC), the main psychoactive component of marijuana, binds to both CB1 and CB2 receptors. Δ⁹-THC is widely reported to increase appetite and food intake (hyperphagia) in humans and in animals. This hyperphagic effect is largely blocked by pretreatment with selective CB1 receptor blockers (i.e., CB1 blockers), strongly supporting the belief that CB1 receptor activation mediates the hyperphagic effect of Δ⁹-THC (Endocrine Reviews 2006, 27, 73).

The CB1 receptor is one of the most abundant and widely distributed G protein-coupled receptors in the mammalian brain. It is known that the appetite-suppressant properties of CB1 antagonists can be mediated through either a direct action with CB1 receptors in brain regions associated with hunger and satiety (e.g., hypothalamus, mesolimbic regions), or a direct action with CB1 receptors in peripheral tissues (e.g., adipose tissue, kidney) or a combination of these central and peripheral actions (J. Clin Invest 2010, 120:2953; Obesity 2011, 19:1325).

Given the wide variety of medical disorders that are related to obesity, it would be beneficial to provide additional advantageous formulations and methods thereof for advantageous delivery that may increase therapeutic activity and/or stability of CB1 receptor blockers.

### SUMMARY OF THE INVENTION

CRB-913 is a cannabinoid receptor type 1 (CB1) inverse agonist (CB1-IA) that is useful for the treatment of weight conditions such as obesity. The present invention is directed to compositions and formulations (e.g., pharmaceutical compositions) of CRB-913, and methods of using CRB-913 for the treatment of weight conditions, e.g., as described herein.

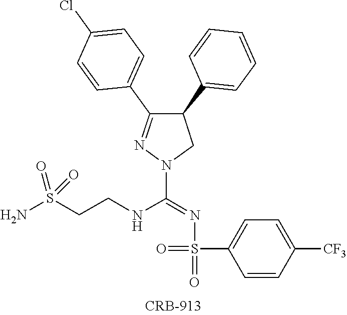 [SMILES: NS(=O)(=O)CCN/C(=N\S(=O)(=O)c1ccc(C(F)(F)F)cc1)N1C[C@H](c2ccccc2)C(c2ccc(Cl)cc2)=N1]

The formulations of CRB-913 can improve the oral bioavailability, solubility, and/or formulation shelf stability of CRB-913.

In one aspect, the present disclosure features a solid dispersion including: (i) substantially amorphous CRB-913; and (ii) a pharmaceutically acceptable polymer, wherein the weight to weight (w/w) ratio of substantially amorphous CRB-913 to pharmaceutically acceptable polymer in the solid dispersion is from 1:10 to 4:1 (e.g., from 1:10 to 4:1; from 3:1 to 7:1, from 2:5 to 5:5, or from 2:5 to 3:5). In certain embodiments, the (w/w) ratio of CRB-913 to the pharmaceutically acceptable polymer in the solid dispersion is from 1:4 to 1:1. In certain embodiments, the (w/w) ratio of CRB-913 to the pharmaceutically acceptable polymer in the solid dispersion is from 2:5 to 3:5. For example, in one embodiment, the (w/w) ratio of substantially amorphous CRB-913 to pharmaceutically acceptable polymer in the solid dispersion is 1:1.

In certain embodiments, the percentage loading of CRB-913 in the solid dispersion is from 10% to 70% (w/w) (e.g., from 10% to 15%, from 12% to 20%, from 15% to 25%, from 25% to 30%, from 25% to 40%, from 25% to 50%, from 25% to 60%, from 25% to 70%, from 30% to 40%, from 30% to 50%, from 30% to 60%, from 30% to 70%, from 40% to 50%, from 40% to 60%, from 40% to 70%, from 45% to 55%, from 48% to 52%, from 50% to 60%, from 50% to 70%, and from 60% to 70% (w/w)). For example, in one embodiment, the percentage loading of CRB-913 in the solid dispersion is from 40% to 60% (w/w). In another embodiment, the percentage loading of CRB-913 in the solid dispersion is 50% (w/w).

In certain embodiments, at least 90% (e.g., at least 95%, 96%, 97%, 98%, 99%, 99.5%, or even 99.9%, such as from 90% to 99.9%, from 90% to 99.5%, from 90% to 99%, from 90% to 98%, from 90% to 97%, from 90% to 96%, from 90% to 95%, from 95% to 99.9%, from 95% to 99.5%, from 95% to 99%, from 95% to 98%, from 95% to 97%, and from 95% to 96%) of the CRB-913 in the solid dispersion is in amorphous form, e.g., as determined by X-ray powder diffraction. The formulations of the present disclosure can exhibit shelf-life stability with respect to the amorphous nature of CRB-913 in the solid dispersions of the present disclosure.

In certain embodiments, the pharmaceutically acceptable polymer in the solid dispersion includes a polymer selected from a cellulose derivative, a polyacrylate, a polyvinyl pyrrolidone, a polyvinyl acetate, a copolymer of a polyvinyl pyrrolidone and a polyvinyl acetate, and combinations thereof.

In further embodiments, the pharmaceutically acceptable polymer in the solid dispersion is a cellulose derivative including cellulose acetate from 10% to 50% by weight (e.g., from 10% to 15%, from 10% to 20%, from 10% to 25%, from 10% to 30%, from 10% to 35%, from 10% to 40%, from 10% to 45%, from 20% to 25%, from 20% to 30%, from 20% to 35%, from 20% to 40%, from 20% to 45%, from 20% to 50%, from 25% to 30%, from 25% to 35%, from 25% to 40%, from 25% to 45%, from 25% to 50%, from 30% to 35%, from 30% to 40%, from 30% to 45%, from 30% to 50%, from 35% to 40%, from 35% to 45%, from 35% to 50%, from 40% to 45%, from 40% to 50%, and from 45% to 50% (w/w)) acetyl. In further embodiments, the cellulose derivative in the solid dispersion is selected from an alkyl cellulose (e.g., methyl cellulose or ethyl cellulose), a hydroxyalkyl cellulose (e.g., hydroxymethyl cellulose; hydroxyethyl cellulose; hydroxypropyl cellulose, such as those having 11% hydroxypropyl substituted or 8% hydroxypropyl substituted; or hydroxybutyl cellulose), a hydroxyalkylalkyl cellulose (e.g., hydroxyethylmethyl cellulose; or hydroxypropylmethyl cellulose (HPMC) having 19-24% methoxyl substituted/7-12% hydroxypropoxyl substituted, 28-30% methoxyl substituted/7-12% hydroxypropoxyl substituted, 23% methoxyl substituted/10% hydroxypropoxyl substituted, 23%-29% methoxyl substituted/8%-9% hydroxypropoxyl substituted, 29% methoxyl/9% hydroxypropoxyl substituted, or 23% methoxyl substituted/6% hydroxypropoxyl substituted), a hydroxyalkylalkyl cellulose ester (e.g., hydroxypropylmethyl cellulose phthalate (HPMCP)), a carboxyalkyl cellulose (e.g., carboxymethyl cellulose or an alkali metal salt thereof, such as a sodium salt), a carboxyalkylalkyl cellulose (e.g., carboxymethylethyl cellulose), and a carboxyalkyl cellulose ester (e.g., carboxymethyl cellulose butyrate, carboxymethyl cellulose propionate, carboxymethyl cellulose acetate butyrate, or carboxymethyl cellulose acetate propionate). In further embodiments, the cellulose derivative in the solid dispersion is cross-linked or copolymerized (e.g., with any polymer described herein). In further embodiments, the cellulose derivative in the solid dispersion is HPMC. In further embodiments, the cellulose derivative in the solid dispersion is HPMC E3. In further embodiments, the cellulose derivative in the solid dispersion is HPMC E5.

In further embodiments, the cellulose acetate in the solid dispersion is cellulose acetate phthalate (CAP) (e.g., having 35% phthalyl/24% acetyl), methylcellulose acetate phthalate, hydroxypropylmethyl cellulose acetate, and hydroxypropylmethyl cellulose acetate succinate (HPMCAS). In certain embodiments, the HPMCAS used in the solid dispersions of the present disclosure comprises 4-15% acetyl by weight, 4-18% succinoyl by weight, 12-28% methoxyl by weight (e.g., 20-26% methoxyl by weight), and 4-28% hydroxypropoxy by weight (e.g., 5-14% hydroxypropoxy by weight). For example, the cellulose acetate polymer in the solid dispersion can be HPMCAS selected from grade L (HPMCAS-L), grade H (HPMCAS-H), or grade M (HPMCAS-M). In some embodiments, the HPMCAS polymer is selected from grade LG (HPMCAS-LG), grade HG (HPMCAS-HG), and grade MG (HPMCAS-MG). The HPMCAS-L polymer may comprise 5-9% acetyl by weight, 14-18% succinoyl by weight, 20-24% methoxyl by weight, and 5-9% hydroxypropoxy by weight. The HPMCAS-M polymer may comprise 7-11% acetyl by weight, 10-14% succinoyl by weight, 21-25% methoxyl by weight, and 5-9% hydroxypropoxy by weight. The HPMCAS-H polymer may comprise 10-14% acetyl by weight, 4-8% succinoyl by weight, 22-26% methoxyl by weight, and 6-10% hydroxypropoxy by weight. In some embodiments, the HPMCAS polymer is HPMCAS-MG.

In some embodiments, the pharmaceutically acceptable polymer in the solid dispersion is a polyacrylate selected from a polymethacrylate, a methacrylate copolymer (e.g., a methacrylic acid-methyl methacrylate copolymer having a 1:1 ratio of free carboxyl groups to ester groups and a 1:2 ratio of free carboxyl groups to ester groups, a dimethylaminoethyl methacrylate-butyl methacrylate-methyl methacrylate copolymer, and a diethylaminoethyl methacrylic acid-methyl methacrylate copolymer), and an ethacrylate copolymer (e.g., a methacrylic acid ethacrylate copolymer having a 50:50 ratio of methacrylic acid to ethacrylate). In further embodiments, the methacrylate copolymer is EUDRAGIT® E-100.

In some embodiments, the pharmaceutically acceptable polymer in the solid dispersion is a polyvinyl acetate selected from a polyvinyl pyrrolidone (e.g., povidone) having a molecular weight of more than 2,000 Da (e.g., 2,500, 9,000, 50,000, or 1,250,000 Da), a polyvinyl acetate ester (e.g., polyvinyl acetate phthalate (PVAP)), and a polyethylene glycol polyvinylacetate copolymer (e.g., polyethylene glycol-polyvinylcaprolactam-polyvinylacetate copolymer).

In other embodiments, the pharmaceutically acceptable polymer in the solid dispersion is a copolymer of a polyvinyl pyrrolidone and a polyvinyl acetate and the copolymer has from 10:90 to 70:30 ratio (e.g., 20:80, 30:70, 40:60, 50:50, and 60:40) of N-vinyl-2-pyrrolidone to vinyl acetate.

In another aspect, the present disclosure features a pharmaceutical composition in a capsule or tablet for oral administration that includes any one of the solid dispersions disclosed herein, optionally in combination with a pharmaceutically acceptable excipient. The pharmaceutical composition may contain from 5 mg to 800 mg (e.g., from 5 mg to 10 mg, from 9 mg to 20 mg, from 20 mg to 40 mg, from 20 mg to 50 mg, from 20 mg to 75 mg, from 20 mg to 100 mg, from 20 mg to 125 mg, from 20 mg to 150 mg, from 20 mg to 175 mg, from 20 mg to 200 mg, from 20 mg to 225 mg, from 30 mg to 40 mg, from 30 mg to 50 mg, from 30 mg to 75 mg, from 30 mg to 100 mg, from 30 mg to 125 mg, from 30 mg to 150 mg, from 30 mg to 175 mg, from 30 mg to 200 mg, from 40 mg to 50 mg, from 40 mg to 75 mg, from 40 mg to 100 mg, from 40 mg to 125 mg, from 40 mg to 150 mg, from 40 mg to 175 mg, from 40 mg to 200 mg, from 50 mg to 75 mg, from 50 mg to 100 mg, from 50 mg to 125 mg, from 50 mg to 150 mg, from 50 mg to 175 mg, from 50 mg to 200 mg, from 60 mg to 75 mg, from 60 mg to 100 mg, from 60 mg to 125 mg, from 60 mg to 150 mg, from 60 mg to 175 mg, from 60 mg to 200 mg, from 70 mg to 75 mg, from 70 mg to 100 mg, from 70 mg to 125 mg, from 70 mg to 150 mg, from 70 mg to 175 mg, from 70 mg to 200 mg, from 80 mg to 100 mg, from 80 mg to 125 mg, from 80 mg to 150 mg, from 80 mg to 175 mg, from 80 mg to 200 mg, from 90 mg to 100 mg, from 90 mg to 125 mg, from 90 mg to 150 mg, from 90 mg to 175 mg, from 90 mg to 200 mg, from 100 mg to 125 mg, from 100 mg to 150 mg, from 100 mg to 175 mg, from 100 mg to 200 mg, from 125 mg to 200 mg, from 150 mg to 200 mg, from 175 mg to 200 mg, from 200 mg to 300 mg, from 225 mg to 300 mg, from 250 mg to 300 mg, from 275 mg to 300 mg, from 300 mg to 325 mg, from 300 mg to 350 mg, from 300 mg to 375 mg, from 400 mg to 500 mg, from 425 mg to 450 mg, from 450 mg to 475 mg, from 500 mg to 600 mg, from 500 mg to 525 mg, from 500 mg to 550 mg, from 500 mg to 575 mg, from 600 mg to 700 mg, from 625 mg to 700 mg, from 650 mg to 700 mg, from 675 mg to 700 mg, from 700 mg to 800 mg, from 725 mg to 800 mg, from 750 mg to 800 mg, and from 775 mg to 800 mg) of substantially amorphous CRB-913. In some embodiments, the pharmaceutical composition contains 5 mg substantially amorphous CRB-913. In some embodiments, the pharmaceutical composition contains 9 mg substantially amorphous CRB-913. In some embodiments, the pharmaceutical composition contains 10 mg substantially amorphous CRB-913. In some embodiments, the pharmaceutical composition contains 15 mg substantially amorphous CRB-913. In some embodiments, the pharmaceutical composition contains 20 mg substantially amorphous CRB-913. In some embodiments, the pharmaceutical composition contains 25 mg substantially amorphous CRB-913. In some embodiments, the pharmaceutical composition contains 30 mg substantially amorphous CRB-913. In some embodiments, the pharmaceutical composition contains 35 mg substantially amorphous CRB-913. In some embodiments, the pharmaceutical composition contains 40 mg substantially amorphous CRB-913. In some embodiments, the pharmaceutical composition contains 45 mg substantially amorphous CRB-913. In some embodiments, the pharmaceutical composition contains 50 mg substantially amorphous CRB-913. In some embodiments, the pharmaceutical composition contains 55 mg substantially amorphous CRB-913. In some embodiments, the pharmaceutical composition contains 60 mg substantially amorphous CRB-913. In some embodiments, the pharmaceutical composition contains 65 mg substantially amorphous CRB-913. In some embodiments, the pharmaceutical composition contains 70 mg substantially amorphous CRB-913. In some embodiments, the pharmaceutical composition contains 75 mg substantially amorphous CRB-913. In some embodiments, the pharmaceutical composition contains 80 mg substantially amorphous CRB-913. In some embodiments, the pharmaceutical composition contains 85 mg substantially amorphous CRB-913. In some embodiments, the pharmaceutical composition contains 90 mg substantially amorphous CRB-913. In some embodiments, the pharmaceutical composition contains 95 mg substantially amorphous CRB-913. In some embodiments, the pharmaceutical composition contains 100 mg substantially amorphous CRB-913. In some embodiments, the pharmaceutical composition contains 105 mg substantially amorphous CRB-913. In some embodiments, the pharmaceutical composition contains 110 mg substantially amorphous CRB-913. In some embodiments, the pharmaceutical composition contains 115 mg substantially amorphous CRB-913. In some embodiments, the pharmaceutical composition contains 120 mg substantially amorphous CRB-913. In some embodiments, the pharmaceutical composition contains 125 mg substantially amorphous CRB-913. In some embodiments, the pharmaceutical composition contains 130 mg substantially amorphous CRB-913. In some embodiments, the pharmaceutical composition contains 135 mg substantially amorphous CRB-913. In some embodiments, the pharmaceutical composition contains 140 mg substantially amorphous CRB-913. In some embodiments, the pharmaceutical composition contains 145 mg substantially amorphous CRB-913. In some embodiments, the pharmaceutical composition contains 150 mg substantially amorphous CRB-913. In some embodiments, the pharmaceutical composition contains 155 mg substantially amorphous CRB-913. In some embodiments, the pharmaceutical composition contains 160 mg substantially amorphous CRB-913. In some embodiments, the pharmaceutical composition contains 165 mg substantially amorphous CRB-913. In some embodiments, the pharmaceutical composition contains 170 mg substantially amorphous CRB-913. In some embodiments, the pharmaceutical composition contains 175 mg substantially amorphous CRB-913. In some embodiments, the pharmaceutical composition contains 180 mg substantially amorphous CRB-913. In some embodiments, the pharmaceutical composition contains 185 mg substantially amorphous CRB-913. In some embodiments, the pharmaceutical composition contains 190 mg substantially amorphous CRB-913. In some embodiments, the pharmaceutical composition contains 195 mg substantially amorphous CRB-913. In some embodiments, the pharmaceutical composition contains 200 mg substantially amorphous CRB-913. In some embodiments, the pharmaceutical composition contains 205 mg substantially amorphous CRB-913. In some embodiments, the pharmaceutical composition contains 210 mg substantially amorphous CRB-913. In some embodiments, the pharmaceutical composition contains 215 mg substantially amorphous CRB-913. In some embodiments, the pharmaceutical composition contains 220 mg substantially amorphous CRB-913. In some embodiments, the pharmaceutical composition contains 225 mg substantially amorphous CRB-913. In some embodiments, the pharmaceutical composition contains 230 mg substantially amorphous CRB-913. In some embodiments, the pharmaceutical composition contains 235 mg substantially amorphous CRB-913. In some embodiments, the pharmaceutical composition contains 240 mg substantially amorphous CRB-913. In some embodiments, the pharmaceutical composition contains 245 mg substantially amorphous CRB-913. In some embodiments, the pharmaceutical composition contains 250 mg substantially amorphous CRB-913. In some embodiments, the pharmaceutical composition contains 255 mg substantially amorphous CRB-913. In some embodiments, the pharmaceutical composition contains 260 mg substantially amorphous CRB-913. In some embodiments, the pharmaceutical composition contains 265 mg substantially amorphous CRB-913. In some embodiments, the pharmaceutical composition contains 270 mg substantially amorphous CRB-913. In some embodiments, the pharmaceutical composition contains 275 mg substantially amorphous CRB-913. In some embodiments, the pharmaceutical composition contains 280 mg substantially amorphous CRB-913. In some embodiments, the pharmaceutical composition contains 285 mg substantially amorphous CRB-913. In some embodiments, the pharmaceutical composition contains 290 mg substantially amorphous CRB-913. In some embodiments, the pharmaceutical composition contains 295 mg substantially amorphous CRB-913. In some embodiments, the pharmaceutical composition contains 300 mg substantially amorphous CRB-913. In some embodiments, the pharmaceutical composition contains 305 mg substantially amorphous CRB-913. In some embodiments, the pharmaceutical composition contains 310 mg substantially amorphous CRB-913. In some embodiments, the pharmaceutical composition contains 315 mg substantially amorphous CRB-913. In some embodiments, the pharmaceutical composition contains 320 mg substantially amorphous CRB-913. In some embodiments, the pharmaceutical composition contains 325 mg substantially amorphous CRB-913. In some embodiments, the pharmaceutical composition contains 330 mg substantially amorphous CRB-913. In some embodiments, the pharmaceutical composition contains 335 mg substantially amorphous CRB-913. In some embodiments, the pharmaceutical composition contains 340 mg substantially amorphous CRB-913. In some embodiments, the pharmaceutical composition contains 345 mg substantially amorphous CRB-913. In some embodiments, the pharmaceutical composition contains 350 mg substantially amorphous CRB-913. In some embodiments, the pharmaceutical composition contains 355 mg substantially amorphous CRB-913. In some embodiments, the pharmaceutical composition contains 360 mg substantially amorphous CRB-913. In some embodiments, the pharmaceutical composition contains 365 mg substantially amorphous CRB-913. In some embodiments, the pharmaceutical composition contains 370 mg substantially amorphous CRB-913. In some embodiments, the pharmaceutical composition contains 375 mg substantially amorphous CRB-913. In some embodiments, the pharmaceutical composition contains 380 mg substantially amorphous CRB-913. In some embodiments, the pharmaceutical composition contains 385 mg substantially amorphous CRB-913. In some embodiments, the pharmaceutical composition contains 390 mg substantially amorphous CRB-913. In some embodiments, the pharmaceutical composition contains 395 mg substantially amorphous CRB-913. In some embodiments, the pharmaceutical composition contains 400 mg substantially amorphous CRB-913. In some embodiments, the pharmaceutical composition contains 405 mg substantially amorphous CRB-913. In some embodiments, the pharmaceutical composition contains 410 mg substantially amorphous CRB-913. In some embodiments, the pharmaceutical composition contains 415 mg substantially amorphous CRB-913. In some embodiments, the pharmaceutical composition contains 420 mg substantially amorphous CRB-913. In some embodiments, the pharmaceutical composition contains 425 mg substantially amorphous CRB-913. In some embodiments, the pharmaceutical composition contains 430 mg substantially amorphous CRB-913. In some embodiments, the pharmaceutical composition contains 435 mg substantially amorphous CRB-913. In some embodiments, the pharmaceutical composition contains 440 mg substantially amorphous CRB-913. In some embodiments, the pharmaceutical composition contains 445 mg substantially amorphous CRB-913. In some embodiments, the pharmaceutical composition contains 450 mg substantially amorphous CRB-913. In some embodiments, the pharmaceutical composition contains 455 mg substantially amorphous CRB-913. In some embodiments, the pharmaceutical composition contains 460 mg substantially amorphous CRB-913. In some embodiments, the pharmaceutical composition contains 470 mg substantially amorphous CRB-913. In some embodiments, the pharmaceutical composition contains 475 mg substantially amorphous CRB-913. In some embodiments, the pharmaceutical composition contains 480 mg substantially amorphous CRB-913. In some embodiments, the pharmaceutical composition contains 485 mg substantially amorphous CRB-913. In some embodiments, the pharmaceutical composition contains 490 mg substantially amorphous CRB-913. In some embodiments, the pharmaceutical composition contains 495 mg substantially amorphous CRB-913. In some embodiments, the pharmaceutical composition contains 500 mg substantially amorphous CRB-913. In some embodiments, the pharmaceutical composition contains 505 mg substantially amorphous CRB-913. In some embodiments, the pharmaceutical composition contains 510 mg substantially amorphous CRB-913. In some embodiments, the pharmaceutical composition contains 515 mg substantially amorphous CRB-913. In some embodiments, the pharmaceutical composition contains 520 mg substantially amorphous CRB-913. In some embodiments, the pharmaceutical composition contains 525 mg substantially amorphous CRB-913. In some embodiments, the pharmaceutical composition contains 530 mg substantially amorphous CRB-913. In some embodiments, the pharmaceutical composition contains 535 mg substantially amorphous CRB-913. In some embodiments, the pharmaceutical composition contains 540 mg substantially amorphous CRB-913. In some embodiments, the pharmaceutical composition contains 545 mg substantially amorphous CRB-913. In some embodiments, the pharmaceutical composition contains 550 mg substantially amorphous CRB-913. In some embodiments, the pharmaceutical composition contains 555 mg substantially amorphous CRB-913. In some embodiments, the pharmaceutical composition contains 560 mg substantially amorphous CRB-913. In some embodiments, the pharmaceutical composition contains 565 mg substantially amorphous CRB-913. In some embodiments, the pharmaceutical composition contains 570 mg substantially amorphous CRB-913. In some embodiments, the pharmaceutical composition contains 575 mg substantially amorphous CRB-913. In some embodiments, the pharmaceutical composition contains 580 mg substantially amorphous CRB-913. In some embodiments, the pharmaceutical composition contains 585 mg substantially amorphous CRB-913. In some embodiments, the pharmaceutical composition contains 590 mg substantially amorphous CRB-913. In some embodiments, the pharmaceutical composition contains 595 mg substantially amorphous CRB-913. In some embodiments, the pharmaceutical composition contains 600 mg substantially amorphous CRB-913. In some embodiments, the pharmaceutical composition contains 605 mg substantially amorphous CRB-913. In some embodiments, the pharmaceutical composition contains 610 mg substantially amorphous CRB-913. In some embodiments, the pharmaceutical composition contains 615 mg substantially amorphous CRB-913. In some embodiments, the pharmaceutical composition contains 620 mg substantially amorphous CRB-913. In some embodiments, the pharmaceutical composition contains 625 mg substantially amorphous CRB-913. In some embodiments, the pharmaceutical composition contains 630 mg substantially amorphous CRB-913. In some embodiments, the pharmaceutical composition contains 635 mg substantially amorphous CRB-913. In some embodiments, the pharmaceutical composition contains 640 mg substantially amorphous CRB-913. In some embodiments, the pharmaceutical composition contains 645 mg substantially amorphous CRB-913. In some embodiments, the pharmaceutical composition contains 650 mg substantially amorphous CRB-913. In some embodiments, the pharmaceutical composition contains 655 mg substantially amorphous CRB-913. In some embodiments, the pharmaceutical composition contains 660 mg substantially amorphous CRB-913. In some embodiments, the pharmaceutical composition contains 665 mg substantially amorphous CRB-913. In some embodiments, the pharmaceutical composition contains 670 mg substantially amorphous CRB-913. In some embodiments, the pharmaceutical composition contains 675 mg substantially amorphous CRB-913. In some embodiments, the pharmaceutical composition contains 680 mg substantially amorphous CRB-913. In some embodiments, the pharmaceutical composition contains 685 mg substantially amorphous CRB-913. In some embodiments, the pharmaceutical composition contains 690 mg substantially amorphous CRB-913. In some embodiments, the pharmaceutical composition contains 695 mg substantially amorphous CRB-913. In some embodiments, the pharmaceutical composition contains 700 mg substantially amorphous CRB-913. In some embodiments, the pharmaceutical composition contains 705 mg substantially amorphous CRB-913. In some embodiments, the pharmaceutical composition contains 710 mg substantially amorphous CRB-913. In some embodiments, the pharmaceutical composition contains 715 mg substantially amorphous CRB-913. In some embodiments, the pharmaceutical composition contains 720 mg substantially amorphous CRB-913. In some embodiments, the pharmaceutical composition contains 725 mg substantially amorphous CRB-913. In some embodiments, the pharmaceutical composition contains 730 mg substantially amorphous CRB-913. In some embodiments, the pharmaceutical composition contains 735 mg substantially amorphous CRB-913. In some embodiments, the pharmaceutical composition contains 740 mg substantially amorphous CRB-913. In some embodiments, the pharmaceutical composition contains 745 mg substantially amorphous CRB-913. In some embodiments, the pharmaceutical composition contains 750 mg substantially amorphous CRB-913. In some embodiments, the pharmaceutical composition contains 755 mg substantially amorphous CRB-913. In some embodiments, the pharmaceutical composition contains 760 mg substantially amorphous CRB-913. In some embodiments, the pharmaceutical composition contains 765 mg substantially amorphous CRB-913. In some embodiments, the pharmaceutical composition contains 770 mg substantially amorphous CRB-913. In some embodiments, the pharmaceutical composition contains 775 mg substantially amorphous CRB-913. In some embodiments, the pharmaceutical composition contains 780 mg substantially amorphous CRB-913. In some embodiments, the pharmaceutical composition contains 785 mg substantially amorphous CRB-913. In some embodiments, the pharmaceutical composition contains 790 mg substantially amorphous CRB-913. In some embodiments, the pharmaceutical composition contains 795 mg substantially amorphous CRB-913. In some embodiments, the pharmaceutical composition contains 800 mg substantially amorphous CRB-913.

In some embodiments, the pharmaceutical composition further includes a plasticizer. Exemplary plasticizers include, but are not limited to, a polyalkylene oxide (e.g., polyethylene glycols, such as PEG 300, PEG 400, PEG 4000, and PEG 8000, and polypropylene glycols), a copolymer of ethylene oxide and propylene oxide (e.g., ethoxylated propoxylated block copolymers having the formula H(OCH₂CH₂)ₐ(OCHCH₃CH₂)_{b}(OCH₂CH₂)ₐOH, in which a is between 10 and 150 and b is between 10 and 100 (e.g., a is 12 and b is 20, a is 38 and b is 29, a is 80 and b is 27, a is 64 and b is 37, a is 141 and b is 44, a is 49 and b is 57, and a is 101 and b is 56), and a polyethoxylated glyceryl ester (e.g., polyoxyl 35 castor oil and polyoxyl 40 castor oil having 40-45 moles of ethylene oxide).

In some embodiments, the pharmaceutical composition further includes pharmaceutically acceptable binders, such as starch, gelatin, natural sugars (e.g., glucose or beta-lactose), corn sweeteners, natural and synthetic gums (e.g., acacia, tragacanth, or sodium alginate), carboxymethylcellulose, polyethylene glycol, and waxes.

In some embodiments, the pharmaceutical composition further includes a filler (e.g., sucrose; sorbitol; mannitol; microcrystalline cellulose; a starch, e.g., potato starch; kaolin, calcium carbonate; sodium chloride; lactose; calcium phosphate; calcium sulphate; and sodium phosphate).

In some embodiments, the pharmaceutical composition further includes a pharmaceutically acceptable carrier. Exemplary pharmaceutically acceptable carriers include, but are not limited to sugars, starches, celluloses, powdered tragacanth, malt, gelatin, talc, and vegetable oils.

In some embodiments, the pharmaceutical composition further includes a pharmaceutically acceptable excipient, e.g., lactose monohydrate, such as granulated alpha-lactose monohydrate (e.g., Tablettose 80); fumed silica, such as Aerosil; magnesium stearate, croscarmellose sodium, colloidal silicon dioxide, and microcrystalline cellulose, such as AVICEL® PH101 and AVICEL® PH102; a colorant; a flavoring agent; a plasticizer; a humectant; a buffering agent; an antioxidant; a coating or a film former; a compression aid; an emollient; an emulsifier; a fragrance; a preservative; a printing ink; a sorbent; a suspending or dispersing agent; a sweetener; and waters of hydration. In some embodiments, the pharmaceutical composition further includes croscarmellose sodium. In some embodiments, the pharmaceutical composition further includes magnesium stearate. In some embodiments, the pharmaceutical composition further includes fumed silica. In some embodiments, the pharmaceutical composition further includes granulated alpha-lactose monohydrate. In some embodiments, the pharmaceutical composition further includes microcrystalline cellulose. In some embodiments, the microcrystalline cellulose is microcrystalline cellulose PH101. In some embodiments, the microcrystalline cellulose is microcrystalline cellulose PH102. In some embodiments, the pharmaceutical composition further includes croscarmellose sodium, magnesium stearate, fumed silica, lactose monohydrate, microcrystalline cellulose PH101, microcrystalline cellulose PH102, lactose, sodium lauryl sulphate (i.e., sodium dodecyl sulphate), and colloidal silicon dioxide.

In some embodiments, the pharmaceutical composition further includes a disintegrant. Exemplary disintegrants include, but are not limited to sodium starch glycolate, crospovidone, cross-linked alginic acid, crosslinked starch, crosslinked alginate sodium, carmellose, carmellose calcium, croscarmellose sodium, glycerin fatty acid ester, low-substituted sodium carboxymethyl starch, and partially pregelatinized starch.

In some embodiments, the pharmaceutical composition further includes a wetting agent. Exemplary wetting agents include, but are not limited to pluronics, PEG, sorbitan esters, and polysorbates such as polysorbate 20, polysorbate 80 (polyoxyethylene (20) sorbitan monooleate).

In some embodiments, the pharmaceutical composition further includes a lubricant. Exemplary lubricants include, but are not limited to glyceryl behenate, glyceryl behaptate, sodium stearyl fumarate, stearic acid, magnesium stearate, calcium stearate, sodium stearate, hydrogenated vegetable oils, colloidal silica, talc, waxes, boric acid, sodium benzoate, sodium acetate, sodium fumarate, sodium chloride, DL-leucine, polyethylene glycol, sodium oleate, sodium lauryl sulphate (i.e., sodium dodecyl sulphate), and magnesium lauryl sulphate.

In some embodiments, the pharmaceutical composition further includes a glidant. Exemplary glidants include those selected from silicon dioxide (e.g., colloidal), hydrated sodium sulfoaluminate, and talc.

In some embodiments, the pharmaceutical composition further includes a surfactant. Exemplary surfactants include those selected from a polyethoxylated ester of one or more fatty acids (e.g., sodium lauryl sulphate (SLS) (i.e., sodium dodecyl sulphate (SDS)), sodium laureth sulphate), a polyethoxylated alkyl ether, a polyethoxylated glyceryl ester, a polyoxyethylene glyceryl ester of one or more fatty acids, a sorbitan ester, a polyethoxylated sorbitan ester, a polyethoxylated vitamin analog (e.g., a TPGS such as D-alpha-tocopheryl PEG 1000 succinate), and an ethoxylated propoxylated block copolymer.

In other embodiments, the solid dispersion in the pharmaceutical composition is formed by spray drying or hot melt extruding a liquid mixture including CRB-913, the pharmaceutically acceptable polymer (e.g., any one of the pharmaceutically acceptable polymers disclosed herein), a pharmaceutically acceptable solvent (e.g., acetone, dichloromethane, methanol, and/or water), and pharmaceutically acceptable surfactant (e.g., sodium lauryl sulphate or sodium dodecyl sulphate (SDS)). In some embodiments, the polymer is HPMCAS-H, HPMCAS-L, HPMCAS-M, HPMCAS-HG, HPMCAS-LG, HPMCAS-MG, polyvinyl pyrrolidone, polyvinyl pyrrolidone/vinyl acetate, HPMC E3, HPMC E5, or EUDRAGIT® E-100. In some embodiments, the solid dispersion in the pharmaceutical composition is formed by spray drying. In other embodiments, the solid dispersion in the pharmaceutical composition is formed by other methods known in the art, e.g., fluid bed bead layering, solvent/anti-solvent precipitation, or wet granulation.

In other embodiments, the pharmaceutical composition is formulated as a capsule (e.g., a hard hydroxy propyl methylcellulose capsule, and hard gelatin capsule) wherein the capsule includes a powder including the solid dispersion. In some embodiments, the pharmaceutical composition is formulated as a tablet.

In other embodiments, the pharmaceutical composition includes a solid dispersion including 1:1 (w/w) CRB-913/HPMCAS-MG (i.e., 50:50 (w/w) CRB-913/HPMCAS-MG).

In another aspect, the present disclosure features a method of preparing any one of the solid dispersions disclosed herein. The method includes: (i) dissolving CRB-913 and a pharmaceutically acceptable polymer (e.g., any one of the pharmaceutically acceptable polymers disclosed herein) in an organic solvent (e.g., one or more of acetone, methanol, ethanol, dichloromethane, and tetrahydrofuran) to form a solution; and (ii) spray drying said solution to form the solid dispersion. In some embodiments, the method includes spray drying the solution from step (i) to form the solid dispersion. In further embodiments, the organic solvent is an organic solvent with a boiling point of less than 150 °C.

In other embodiments, the present disclosure features a method of treating obesity in a subject in need of such treatment. The method includes orally administering to the subject an effective amount of the pharmaceutical composition.

In other embodiments, the present disclosure features a method of treating a co-morbidity of obesity in a subject in need of such treatment. The method includes orally administering to the subject an effective amount of the pharmaceutical composition. In some embodiments, the co-morbidity of obesity is diabetes, dyslipidemia, Metabolic Syndrome, dementia, a cardiovascular disease, or a hepatic disease. In some embodiments, the co-morbidity of obesity is hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; or increased incidence of malignancy of the ovaries, cervix, uterus, breasts, prostrate, or gallbladder.

In further embodiments, the present disclosure features a method where the pharmaceutical composition is orally administered once, twice, or three times per day. In some embodiments, the pharmaceutical composition is orally administered once daily with 5 mg to 800 mg of CRB-913 in a pharmaceutical composition.

In further embodiments, any of the above methods of treating further includes administering to the subject a second therapeutic agent. In some embodiments, the second therapeutic agent is a PPAR-γ agonist, a biguanide, insulin or an insulin mimetic, a sulfonylurea, an α-glucosidase inhibitor, an HMG-CoA reductase inhibitor, a sequestrant, nicotinyl alcohol, nicotinic acid or a salt thereof, a PPAR-α agonist, an inhibitor of cholesterol absorption, an acyl CoA: cholesterol acyltransferase inhibitor, probucol, a PPAR-α/γ agonist, an ileal bile acid transporter inhibitor, an insulin receptor activator, a dipeptidyl peptidase IV inhibitor, exenatide, pramlintide, an FBPase inhibitor, a glucagon receptor antagonist, glucagon-like peptide 1, a glucagon-like peptide 1 receptor agonist, a growth hormone secretagogue, a growth hormone secretagogue receptor agonist, a growth hormone secretagogue receptor antagonist, a melanocortin agonist, a melanocortin 4 receptor agonist, a beta-3 agonist, a serotonin receptor 2C agonist, an orexin antagonist, a melanin concentrating hormone 1 antagonist, a melanin concentrating hormone 2 agonist, a melanin concentrating hormone 2 antagonist, a galanin antagonist, a CCK agonist, a CCK-A agonist, a corticotropin-releasing hormone agonist, an NPY 5 antagonist, an NPY 1 antagonist, a histamine receptor-3 modulator, a histamine receptor-3 blocker, a β-hydroxy steroid dehydrogenase-1 inhibitor, a phosphodiesterase inhibitor, a phosphodiesterase-3B inhibitor, a norepinephrine transport inhibitor, a non-selective serotonin/norepinephrine transport inhibitor, a ghrelin antagonist, a leptin derivative, a bombesin receptor subtype 3 agonist, a ciliary neurotrophic factor or a derivative thereof, a monoamine reuptake inhibitor, an uncoupling protein-1 activator, an uncoupling protein-2 activator, an uncoupling protein-3 activator, a thyroid hormone beta agonist, a fatty acid synthase inhibitor, a diacylglycerol acetyltransferase 2 inhibitor, an acetyl-CoA carboxylase-2 inhibitor, a glucocorticoid antagonist, an acyl-estrogen, a lipase inhibitor, a fatty acid transporter inhibitor, a dicarboxylate transporter inhibitor, a glucose transporter inhibitor, a sodium-glucose co-transporter, a phosphate transporter inhibitor, a serotonin reuptake inhibitor, a thiazolidinedione, Metformin, Topiramate, an opiate antagonist, a non-selective transport inhibitor, a MAO inhibitor, glucose-dependent insulinotropic polypeptide (GIP) modulator, or an amylin receptor agonist. In some embodiments, the second therapeutic agent is a glucagon-like peptide 1 receptor agonist (e.g., liraglutide, semaglutide, exenatide, lixisenatide, dulaglutide, or tirzepatide). In some embodiments, the second therapeutic agent is semaglutide. In some embodiments, the second therapeutic agent is tirzepatide. In other embodiments, the second therapeutic agent is a glucose-dependent insulinotropic polypeptide (GIP) modulator, either as an agonist (e.g., tirzepatide) or antagonist (e.g., AMG133). In some embodiments, the second therapeutic agent is an amylin receptor agonist (e.g., pramlintide or cagrilintide).

In another aspect, the present disclosure features a solid dispersion composition including:
at least one polymeric component (e.g., as described herein); and
substantially amorphous CRB-913:

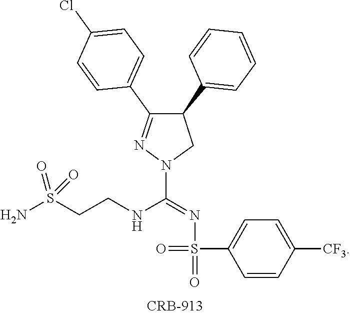 [SMILES: NS(=O)(=O)CCN/C(=N\S(=O)(=O)c1ccc(C(F)(F)F)cc1)N1C[C@H](c2ccccc2)C(c2ccc(Cl)cc2)=N1]

In some embodiments, the percentage loading of CRB-913 is from 20% w/w to 80% w/w. In some embodiments, the percentage loading of CRB-913 is 50%±5% w/w. In some particular embodiments, the percentage loading of CRB-913 is 50%±1% w/w.

In some embodiments, the polymeric component includes a cellulose derivative, a polyacrylate, a polyvinyl pyrrolidone, a polyvinyl acetate, a polyether, or a copolymer thereof, or any combination thereof. In some embodiments, the polymeric component includes Methyl cellulose (MC), Ethyl cellulose (EC), Hydroxyethyl cellulose (HEC), Hydroxypropyl cellulose (HPC), Hydroxypropylmethyl cellulose (HPMC), Hydroxyethylmethyl cellulose (HEMC), hydroxypropylmethyl cellulose succinate (HPMCAS), carboxymethylethylcellulose, sodium carboxymethylcellulose, potassium carboxymethyl cellulose, cellulose acetate succinate, cellulose acetate phthalate, hydroxypropylmethyl cellulose phthalate, polyacrylic acid copolymer, poly(meth)acrylic acid, poly(hydroxyalkyl acrylate), poly (hydroxyalkyl methacrylate), Polyvinylpyrrolidone (PVP), Polyethylene glycol (PEG), Polyvinyl alcohol (PVA), polyvinylcaprolactam, amino methacrylate copolymer, or any combination thereof.

In some embodiments, the polymeric component includes PEG, PVP, dimethylaminoethyl methacrylate, butyl methacrylate, methyl methacrylate, HPMCAS, or a copolymer thereof, or any combination thereof.

In some embodiments, the polymeric component is HPMCAS. In some embodiments, the HPMCAS polymer is selected from grade L (HPMCAS-L), grade H (HPMCAS-H), and grade M (HPMCAS-M). In some specific embodiments, the HPMCAS polymer is HPMCAS-M.

In some embodiments, the percentage loading of the polymer component is from 20% w/w to 80% w/w. In some embodiments, the percentage loading of the polymer component is 50%±5% w/w. In some particular embodiments, the percentage loading of the polymer component is 50%±1% w/w.

In some embodiments, the composition includes:
50% w/w HPMCAS-M; and
50% w/w CRB-913.

In some embodiments, the composition is prepared by spray drying.

In another aspect, the disclosure features a pharmaceutical composition including:
CRB-913:

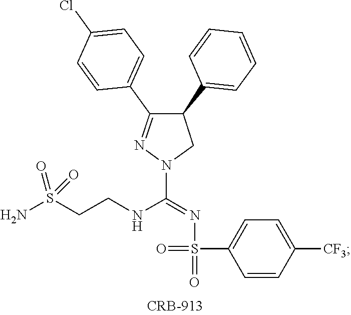 [SMILES: NS(=O)(=O)CCN/C(=N\S(=O)(=O)c1ccc(C(F)(F)F)cc1)N1C[C@H](c2ccccc2)C(c2ccc(Cl)cc2)=N1]

and
at least one pharmaceutically acceptable excipient selected from filler, binder, disintegrant, lubricant, and glidant,
wherein the pharmaceutical composition is in the form of a tablet.

In some embodiments, the composition includes a CRB-913 solid dispersion composition, e.g., as described here. In some embodiments, the composition includes from 5% to 60% w/w of the solid dispersion composition.

In some embodiments, the composition includes 25% to 75% w/w of the at least one filler. In some embodiments, the at least one filler includes microcrystalline cellulose, lactose monohydrate, or a combination thereof.

In some embodiments, the composition includes 3% to 9% w/w of the at least one disintegrant. In some embodiments, the disintegrant is croscarmellose sodium.

In some embodiments, the composition includes at least one glidant. In some embodiments, the composition includes 0.3% to 3% w/w of the at least one glidant.

In some embodiments, the composition includes at least one lubricant. In some embodiments, the composition includes 0.1% to 2% w/w of the at least one lubricant.

In some specific embodiments, the composition includes:
25% to 75% w/w of at least one filler (e.g., 25% to 75% w/w of one or more filers);
3% to 9% w/w disintegrant;
0.3% to 3% w/w glidant; and
0.1% to 2% w/w lubricant.

In some specific embodiments, the composition includes:
filler comprising microcrystalline cellulose, lactose monohydrate, or a combination thereof;
disintegrant comprising croscarmellose sodium;
glidant comprising silicon dioxide; and
lubricant comprising magnesium stearate.

In some specific embodiments, the composition includes:
25% to 75% w/w filler, wherein the filler is microcrystalline cellulose, lactose monohydrate, or a combination thereof
3% to 9% w/w croscarmellose sodium;
0.3% to 3% silicon dioxide; and
0.1% to 2% w/w magnesium stearate.

In some specific embodiments, the composition includes:
36%±1% w/w microcrystalline cellulose;
36%±1% w/w lactose monohydrate;
6%±1% w/w croscarmellose sodium;
1%±0.5% w/w silicon dioxide; and
0.5%±0.1% w/w magnesium stearate.

In some specific embodiments, the composition includes:
21%±1% w/w microcrystalline cellulose;
21%±1% w/w lactose monohydrate;
6%±1% w/w croscarmellose sodium;
1%±0.5% w/w silicon dioxide; and
0.5%±0.1% w/w magnesium stearate.

In some embodiments, the composition includes from 9±5 mg to 600±5 mg of CRB-913. In some specific embodiments, the composition includes from 10±2 mg to 150±2 mg of CRB-913.

In some embodiments, the composition includes 20±2 mg of CRB-913.

In some embodiments, the composition includes 40±2 mg of CRB-913.

In some embodiments, the composition includes 50±2 mg of CRB-913.

In some embodiments, the composition includes 60±2 mg of CRB-913.

In some embodiments, the composition includes 80±2 mg of CRB-913.

In some embodiments, the composition includes 100±2 mg of CRB-913.

In some embodiments, the composition includes 150±2 mg of CRB-913.

In some embodiments, the composition includes 200±2 mg of CRB-913.

In some embodiments, the composition includes 300±2 mg of CRB-913.

In some embodiments, the composition includes 400±2 mg of CRB-913.

In some embodiments, the composition includes 600±2 mg of CRB-913.

In some embodiments, the tablet includes a film coating (e.g., as described herein). In some embodiments, the film coating comprises polyvinyl alcohol (PVA), polyethylene glycol (PEG), or a combination thereof. In some embodiments, the film coating includes PVA, PEG/Macrogol, titanium dioxide, talc, ferric oxide red, ferric oxide yellow, or ferrosoferric oxide NF/black iron oxide, or any combination thereof. In some embodiments, the film coating includes PVA, PEG/Macrogol, titanium dioxide, talc, ferric oxide red, ferric oxide yellow, and ferrosoferric oxide NF/black iron oxide.

In another aspect, the present disclosure features a solid dispersion composition, e.g., as described herein, for use in reducing weight in a subject in need thereof.

In another aspect, the present disclosure features a pharmaceutical composition, e.g., as described herein, for use in reducing weight in a subject in need thereof.

In another aspect, the present disclosure features a solid dispersion composition, e.g., as described herein, for use in treating a weight condition or a co-morbidity thereof in a subject in need thereof.

In another aspect, the present disclosure features a pharmaceutical composition, e.g., as described herein, for use in treating a weight condition or a co-morbidity thereof in a subject in need thereof.

In another aspect, the present disclosure features a method of treating a weight condition (e.g., as described herein) in a subject in need thereof, the method includes orally administering to the subject a total daily dose of 9±5 mg to 600±5 mg of CRB-913 (e.g., 10±5 mg, 15±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5 mg, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, 300±5 mg, 310±5 mg, 320±5 mg, 330±5 mg, 340±5 mg, 350±5 mg, 360±5 mg, 370±5 mg, 380±5 mg, 390±5 mg, 400±5 mg, 410±5 mg, 420±5 mg, 430±5 mg, 440±5 mg, 450±5 mg, 460±5 mg, 470±5 mg, 480±5 mg, 490±5 mg, 500±5 mg, 510±5 mg, 520±5 mg, 530±5 mg, 540±5 mg, 550±5 mg, 560±5 mg, 570±5 mg, 580±5 mg, 590±5 mg, or 600±5 mg).

In another aspect, the present disclosure features a method of treating a weight-related condition (e.g., as described herein) in a subject in need thereof, the method includes orally administering to the subject a total daily dose of 9±5 mg to 600±5 mg of CRB-913 (e.g., 10±5 mg, 15±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5 mg, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, 300±5 mg, 310±5 mg, 320±5 mg, 330±5 mg, 340±5 mg, 350±5 mg, 360±5 mg, 370±5 mg, 380±5 mg, 390±5 mg, 400±5 mg, 410±5 mg, 420±5 mg, 430±5 mg, 440±5 mg, 450±5 mg, 460±5 mg, 470±5 mg, 480±5 mg, 490±5 mg, 500±5 mg, 510±5 mg, 520±5 mg, 530±5 mg, 540±5 mg, 550±5 mg, 560±5 mg, 570±5 mg, 580±5 mg, 590±5 mg, or 600±5 mg).

In another aspect, the present disclosure features a method of maintaining a BMI in a subject previously treated for a weight condition (e.g., as described herein), the method includes orally administering to the subject a total daily dose of 9±5 mg to 600±5 mg of CRB-913 (e.g., 10±5 mg, 15±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5 mg, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, 300±5 mg, 310±5 mg, 320±5 mg, 330±5 mg, 340±5 mg, 350±5 mg, 360±5 mg, 370±5 mg, 380±5 mg, 390±5 mg, 400±5 mg, 410±5 mg, 420±5 mg, 430±5 mg, 440±5 mg, 450±5 mg, 460±5 mg, 470±5 mg, 480±5 mg, 490±5 mg, 500±5 mg, 510±5 mg, 520±5 mg, 530±5 mg, 540±5 mg, 550±5 mg, 560±5 mg, 570±5 mg, 580±5 mg, 590±5 mg, or 600±5 mg).

In another aspect, the present disclosure features a method of maintaining a BMI in a subject previously treated for a weight-related condition (e.g., as described herein), the method includes orally administering to the subject a total daily dose of 9±5 mg to 600±5 mg of CRB-913 (e.g., 10±5 mg, 15±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5 mg, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, 300±5 mg, 310±5 mg, 320±5 mg, 330±5 mg, 340±5 mg, 350±5 mg, 360±5 mg, 370±5 mg, 380±5 mg, 390±5 mg, 400±5 mg, 410±5 mg, 420±5 mg, 430±5 mg, 440±5 mg, 450±5 mg, 460±5 mg, 470±5 mg, 480±5 mg, 490±5 mg, 500±5 mg, 510±5 mg, 520±5 mg, 530±5 mg, 540±5 mg, 550±5 mg, 560±5 mg, 570±5 mg, 580±5 mg, 590±5 mg, or 600±5 mg).

In some embodiments, the subject previously received a weight loss therapy selected from: GLP-1 receptor agonist, a CB1 inverse agonist, a glucose-dependent insulinotropic polypeptide (GIP) modulator, a dual glucose-dependent insulinotropic polypeptide (GIP) modulator and glucagon-like peptide 1 receptor agonist, a triple agonist for weight loss (e.g. a single drug that activates three different hormone receptors such as GLP-1, GIP, and Glucagon), an amylin receptor agonist, an appetite suppressant, a caloric restriction regimen, gastric bypass, endoscopic sleeve, intragastric balloon, or a fat-absorption inhibitor.

In some embodiments, the weight condition or weight related condition is a metabolic/weight issue (e.g. obesity, overweight, weight gain, binge eating disorder, bulimia, antipsychotic medication-induced weight gain, delaying diabetes progress), non-metabolic/weight issue (e.g., anxiety and depression in schizophrenia), weight management, weight loss (e.g. appetite suppression, satiety), sleep apnea, cardiovascular disease (e.g. MACE, high blood pressure, heart failure, low HDL-C), NASH/MASH/NAFLD (e.g. fatty liver), Alzheimer's, gastrointestinal conditions (e.g. increasing/reducing gastric motility, accelerating/delaying gastric emptying), blood sugar/lipids level imbalances (e.g. cholesterol), substance use disorders (alcohol and nicotine), and diabetic nephropathy.

In some embodiments, the weight condition is obesity, overweight, weight gain, binge eating disorder, bulimia, or antipsychotic-induced weight gain.

In some embodiments, the weight condition is obesity.

In some embodiments, the method further includes treating a co-morbidity of obesity. In some embodiments, the co-morbidity of obesity is diabetes, dyslipidemia, Metabolic Syndrome, dementia, a cardiovascular disease, or a hepatic disease. In some embodiments, the co-morbidity of obesity is hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; or increased incidence of malignancy of the ovaries, cervix, uterus, breasts, prostrate, or gallbladder.

In some embodiments, the weight condition is a BMI of ≥27 kg/m².

In some embodiments, the subject suffers from an additional weight-related condition. In some specific embodiments, the subject suffers from type 1 diabetes. In other specific embodiments, the subject suffers from type 2 diabetes.

In another aspect, the present disclosure features a method of treating a weight-related condition (e.g., as described herein) in a subject in need thereof, the method comprising orally administering to the subject a total daily dose of 9±5 mg to 600±5 mg of CRB-913. In some embodiments, the weight-related condition is sleep apnea, cardiovascular disease, Alzheimer's, high cholesterol, or gastrointestinal issues.

In some embodiments, the subject is a human subject.

In some embodiments, the CRB-913 administered is part of a solid dispersion composition or pharmaceutical composition, e.g., a solid dispersion composition or pharmaceutical composition as described herein.

In some embodiments, the total daily dose of CRB-913 administered is 9±2 mg to 150±2 mg (e.g., 10±5 mg, 15±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5 mg, 140±5 mg, or 150±5 mg).

In some embodiments, the total daily dose of CRB-913 administered is 9±2 mg to 300±2 mg (e.g., 10±5 mg, 15±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5 mg, 140±5 mg, 150±5 mg, 110±5 mg, 120±5 mg, 130±5 mg, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, or 300±5 mg).

In some embodiments, the method includes administering a total daily dose of 20±2 mg to 80±2 mg of CRB-913 (e.g., 20±2 mg, 25±5 mg, 30±5 mg, 40±5 mg, 50±5 mg, 60±5 mg, 70±5 mg, 75±5 mg, or 80±2 mg).

In some embodiments, the method includes administering a total daily dose of 70±2 mg to 150±2 mg of CRB-913 (e.g., 70±5 mg, 80±5 mg, 90±5 mg, 100±5 mg, 110±5 mg, 120±5 mg, 130±5 mg, 140±5 mg, 150±5 mg, 110±5 mg, 120±5 mg, 130±5 mg, 140±5 mg, or 150±5 mg).

In some embodiments, the method includes administering a total daily dose of 20±2 mg of CRB-913.

In some embodiments, the method includes administering a total daily dose of 40±2 mg of CRB-913.

In some embodiments, the method includes administering a total daily dose of 60±2 mg of CRB-913.

In some embodiments, the method includes administering a total daily dose of 80±2 mg of CRB-913.

In some embodiments, the method includes administering a total daily dose of 100±2 mg of CRB-913.

In some embodiments, the method includes administering a total daily dose of 120±2 mg of CRB-913.

In some embodiments, the method includes administering a total daily dose of 140±2 mg of CRB-913.

In some embodiments, the method includes administering a total daily dose of 150±2 mg of CRB-913.

In some embodiments, the method includes administering a total daily dose of 300±2 mg of CRB-913.

In some embodiments, the method includes administering a total daily dose of 450±2 mg of CRB-913.

In some embodiments, the method includes administering a dose of CRB-913 at least once a week. In some embodiments, the method includes administering a dose of CRB-913 once a week. In some embodiments, the method includes administering a dose of CRB-913 twice a week. In some embodiments, the method includes administering a dose of CRB-913 three times a week. In some embodiments, each dose individually comprises either 20±2 mg, 40±2 mg, 60±2 mg, 80±2 mg, 100±2 mg, 150±2 mg, 200±2 mg, 300±2 mg, 400±2 mg, or 600±2 mg of CRB-913.

In some embodiments, the method includes administering a dose of CRB-913 at least once a day. In some embodiments, the method includes administering a dose of CRB-913 once a day. In some embodiments, the daily dose includes either 20±2 mg, 40±2 mg, 60±2 mg, 80±2 mg, 100±2 mg, 150±2 mg, 200±2 mg, 300±2 mg, 400±2 mg, or 600±2 mg of CRB-913. In some embodiments, the method includes administering a dose of CRB-913 twice a day. In some embodiments, the method includes administering a dose of CRB-913 three times a day. In some embodiments, each dose individually comprises either 20±2 mg, 40±2 mg, 60±2 mg, 80±2 mg, 100±2 mg, or 150±2 mg of CRB-913.

In some embodiments, the method includes administering a 20±2 mg dose of CRB-913 to the subject once a day.

In some embodiments, the method includes administering a 20±2 mg dose of CRB-913 to the subject twice a day.

In some embodiments, the method includes administering a 20±2 mg dose of CRB-913 to the subject three times a day.

In some embodiments, the method includes administering a 40±2 mg dose of CRB-913 to the subject once a day.

In some embodiments, the method includes administering a 40±2 mg dose of CRB-913 to the subject twice a day.

In some embodiments, the method includes administering a 40±2 mg dose of CRB-913 to the subject three times a day.

In some embodiments, the method includes administering a 50±2 mg dose of CRB-913 to the subject once a day.

In some embodiments, the method includes administering a 50±2 mg dose of CRB-913 to the subject twice a day.

In some embodiments, the method includes administering a 50±2 mg dose of CRB-913 to the subject three times a day.

In some embodiments, the method includes administering a 60±2 mg dose of CRB-913 to the subject once a day.

In some embodiments, the method includes administering a 60±2 mg dose of CRB-913 to the subject twice a day.

In some embodiments, the method includes administering a 60±2 mg dose of CRB-913 to the subject three times a day.

In some embodiments, the method includes administering a 75±2 mg dose of CRB-913 to the subject once a day.

In some embodiments, the method includes administering a 75±2 mg dose of CRB-913 to the subject twice a day.

In some embodiments, the method includes administering a 75±2 mg dose of CRB-913 to the subject three times a day.

In some embodiments, the method includes administering a 80±2 mg dose of CRB-913 to the subject once a day.

In some embodiments, the method includes administering a 80±2 mg dose of CRB-913 to the subject twice a day.

In some embodiments, the method includes administering a 80±2 mg dose of CRB-913 to the subject three times a day.

In some embodiments, the method includes administering a 90±2 mg dose of CRB-913 to the subject once a day.

In some embodiments, the method includes administering a 90±2 mg dose of CRB-913 to the subject twice a day.

In some embodiments, the method includes administering a 90±2 mg dose of CRB-913 to the subject three times a day.

In some embodiments, the method includes administering a 100±2 mg dose of CRB-913 to the subject once a day.

In some embodiments, the method includes administering a 100±2 mg dose of CRB-913 to the subject twice a day.

In some embodiments, the method includes administering a 100±2 mg dose of CRB-913 to the subject three times a day.

In some embodiments, the method includes administering a 120±2 mg dose of CRB-913 to the subject once a day.

In some embodiments, the method includes administering a 120±2 mg dose of CRB-913 to the subject twice a day.

In some embodiments, the method includes administering a 120±2 mg dose of CRB-913 to the subject three times a day.

In some embodiments, the method includes administering a 140±2 mg dose of CRB-913 to the subject once a day.

In some embodiments, the method includes administering a 140±2 mg dose of CRB-913 to the subject twice a day.

In some embodiments, the method includes administering a 140±2 mg dose of CRB-913 to the subject three times a day.

In some embodiments, the method includes administering a 150±2 mg dose of CRB-913 to the subject once a day.

In some embodiments, the method includes administering a 150±2 mg dose of CRB-913 to the subject twice a day.

In some embodiments, the method includes administering a 150±2 mg dose of CRB-913 to the subject three times a day.

In some embodiments, CRB-913 is chronically administered to the subject.

In some embodiments, CRB-913 is administered to the subject in ascending doses (e.g., as described herein).

In some embodiments, the ascending dose administration includes:
(i) an initial administration of an average daily dose over a period of 1 to 24 weeks;
(ii) after (i), administering 1.5 to 4 times the average daily dose over a period of 1 to 24 weeks;
(iii) after (ii), administering 1.5 to 4 times the dose previously administered over a period of 1 to 24 weeks; and
(iv) repeating (iii) until at least the subject is a desired weight (e.g., a BMI >30 kg/m², a BMI >28 kg/m², or a target body weight for the subject).

In some embodiments, the method further includes (v) continuing to administer CRB-913 to maintain the desired weight of the subject.

In some embodiments, the ascending dose administration includes:
(i) an initial administration of an average daily dose over a first period of at least 7 days; and
(ii) following step (i), administration of 1.5 to 4 times the average daily dose over a second period of at least 7 days.

In some embodiments, step (i) includes administering to the subject during the first period an average daily dose of from 9±5 mg to 600±5 mg of CRB-913. In some embodiments, step (i) includes administering to the subject during the first period an average daily dose of from 10±5 mg to 100±5 mg (e.g., 10±5 mg, 15±5 mg, 20±5 mg, 25±5 mg, 30±5 mg, 35±5 mg, 40±5 mg, 45±5 mg, 50±5 mg, 55±5 mg, 60±5 mg, 65±5 mg, 70±5 mg, 75±5 mg, 80±5 mg, 85±5 mg, 90±5 mg, 95±5 mg, or 100±5 mg) of CRB-913. In some embodiments, step (i) includes administering to the subject during the first period an average daily dose of from 10±5 mg to 60±5 mg of CRB-913. In some embodiments, step (i) includes administering to the subject during the first period an average daily dose of from 60±5 mg to 100±5 mg of CRB-913.

In some embodiments, step (ii) includes administering to the subject during the second period an average daily dose of from 20±5 mg to 200±5 mg (e.g., 20±5 mg, 25±5 mg, 30±5 mg, 35±5 mg, 40±5 mg, 45±5 mg, 50±5 mg, 55±5 mg, 60±5 mg, 65±5 mg, 70±5 mg, 75±5 mg, 80±5 mg, 85±5 mg, 90±5 mg, 95±5 mg, 100±5 mg, 105±5 mg, 110±5 mg, 115±5 mg, 120±5 mg, 125±5 mg, 130±5 mg, 135±5 mg, 140±5 mg, 145±5 mg, 150±5 mg, 155±5 mg, 160±5 mg, 165±5 mg, 170±5 mg, 175±5 mg, 180±5 mg, 185±5 mg, 190±5 mg, 195±5 mg, or 200±5 mg) of CRB-913. In some embodiments, step (ii) includes administering to the subject during the second period an average daily dose of from 20±5 mg to 100±5 mg of CRB-913. In some embodiments, step (ii) includes administering to the subject during the second period an average daily dose of from 100±5 mg to 200±5 mg of CRB-913.

In some embodiments, the method further includes: (iii) following step (ii), administration of 1.5 to 8 times the average daily dose over a third period of at least 7 days, wherein the average daily dose over the second period is less than the average daily dose administered over a third period.

In some embodiments, step (iii) includes administering to the subject during the third period an average daily dose of from 40±5 mg to 450±5 mg (e.g., 40±5 mg, 55±5 mg, 60±5 mg, 65±5 mg, 70±5 mg, 75±5 mg, 80±5 mg, 85±5 mg, 90±5 mg, 95±5 mg, 100±5 mg, 105±5 mg, 110±5 mg, 115±5 mg, 120±5 mg, 125±5 mg, 130±5 mg, 135±5 mg, 140±5 mg, 145±5 mg, 150±5 mg, 155±5 mg, 160±5 mg, 165±5 mg, 170±5 mg, 175±5 mg, 180±5 mg, 185±5 mg, 190±5 mg, 195±5 mg, 200±5 mg, 205±5 mg, 210±5 mg, 215±5 mg, 220±5 mg, 225±5 mg, 230±5 mg, 235±5 mg, 240±5 mg, 245±5 mg, 250±5 mg, 255±5 mg, 260±5 mg, 265±5 mg, 270±5 mg, 275±5 mg, 280±5 mg, 285±5 mg, 290±5 mg, 295±5 mg, 300±5 mg, 305±5 mg, 310±5 mg, 315±5 mg, 320±5 mg, 325±5 mg, 330±5 mg, 335±5 mg, 340±5 mg, 345±5 mg, 350±5 mg, 355±5 mg, 360±5 mg, 365±5 mg, 370±5 mg, 375±5 mg, 380±5 mg, 385±5 mg, 390±5 mg, 395±5 mg, 400±5 mg, 405±5 mg, 410±5 mg, 415±5 mg, 420±5 mg, 425±5 mg, 430±5 mg, 435±5 mg, 440±5 mg, 445±5 mg, or 450±5 mg) of CRB-913. In some embodiments, step (iii) includes administering to the subject during the third period an average daily dose of from 40±5 mg to 150±5 mg of CRB-913. In some embodiments, step (iii) includes administering to the subject during the third period an average daily dose of from150±5 mg to 450±5 mg of CRB-913.

In some embodiments, the method further includes: (iv) following step (iii), administration of 4 to 10 times the average daily dose over a fourth period of at least 7 days, wherein the average daily dose over the third period is less than the average daily dose administered over a fourth period.

In some embodiments, step (iv) includes administering to the subject during the fourth period an average daily dose of from 80±5 mg to 600±5 mg (e.g., 80±5 mg, 85±5 mg, 90±5 mg, 95±5 mg, 100±5 mg, 105±5 mg, 110±5 mg, 115±5 mg, 120±5 mg, 125±5 mg, 130±5 mg, 135±5 mg, 140±5 mg, 145±5 mg, 150±5 mg, 155±5 mg, 160±5 mg, 165±5 mg, 170±5 mg, 175±5 mg, 180±5 mg, 185±5 mg, 190±5 mg, 195±5 mg, 200±5 mg, 205±5 mg, 210±5 mg, 215±5 mg, 220±5 mg, 225±5 mg, 230±5 mg, 235±5 mg, 240±5 mg, 245±5 mg, 250±5 mg, 255±5 mg, 260±5 mg, 265±5 mg, 270±5 mg, 275±5 mg, 280±5 mg, 285±5 mg, 290±5 mg, 295±5 mg, 300±5 mg, 305±5 mg, 310±5 mg, 315±5 mg, 320±5 mg, 325±5 mg, 330±5 mg, 335±5 mg, 340±5 mg, 345±5 mg, 350±5 mg, 355±5 mg, 360±5 mg, 365±5 mg, 370±5 mg, 375±5 mg, 380±5 mg, 385±5 mg, 390±5 mg, 395±5 mg, 400±5 mg, 405±5 mg, 410±5 mg, 415±5 mg, 420±5 mg, 425±5 mg, 430±5 mg, 435±5 mg, 440±5 mg, 445±5 mg, 450±5 mg, 455±5 mg, 460±5 mg, 465±5 mg, 470±5 mg, 475±5 mg, 480±5 mg, 485±5 mg, 490±5 mg, 495±5 mg, 500±5 mg, 505±5 mg, 510±5 mg, 515±5 mg, 520±5 mg, 525±5 mg, 530±5 mg, 535±5 mg, 540±5 mg, 545±5 mg, 550±5 mg, 555±5 mg, 560±5 mg, 565±5 mg, 570±5 mg, 575±5 mg, 580±5 mg, 585±5 mg, 590±5 mg, 595±3 mg, or 600±2 mg) of CRB-913. In some embodiments, step (iv) includes administering to the subject during the fourth period an average daily dose of from 80±5 mg to 300±5 mg of CRB-913. In some embodiments, step (iv) includes administering to the subject during the fourth period an average daily dose of from 300±5 mg to 600±5 mg of CRB-913.

In some embodiments, the method further includes administering each does of CRB-913 without food.

In some embodiments, the method further includes administering each dose of CRB-913 with food.

In some embodiments, the method further includes administering each dose of CRB-913 within 0 to 300 minutes (e.g., 0 to 10 minutes, 0 to 30 minutes, 0 to 60 minutes, 0 to 120 minutes, 0 to 240 minutes, 15 to 45 minutes, 30 to 60 minutes, 30 to 120 minutes, 60 to 120 minutes, 60 to 240 minutes, 120 to 240 minutes, or 120 to 300 minutes) after the subject has consumed food.

In some embodiments, the method further includes administering each dose of CRB-913 within 0 to 120 minutes after the subject has consumed food.

In some embodiments, the method further includes administering to the subject a second therapeutic agent. In some embodiments, the second therapeutic agent is a PPAR-γ agonist, a biguanide, insulin or an insulin mimetic, a sulfonylurea, an α-glucosidase inhibitor, an HMG-CoA reductase inhibitor, a sequestrant, nicotinyl alcohol, nicotinic acid or a salt thereof, a PPAR-α agonist, an inhibitor of cholesterol absorption, an acyl CoA: cholesterol acyltransferase inhibitor, probucol, a PPAR-α/γ agonist, an ileal bile acid transporter inhibitor, an insulin receptor activator, a dipeptidyl peptidase IV inhibitor, exenatide, pramlintide, an FBPase inhibitor, a glucagon receptor antagonist, glucagon-like peptide 1, a glucagon-like peptide 1 receptor agonist, a growth hormone secretagogue, a growth hormone secretagogue receptor agonist, a growth hormone secretagogue receptor antagonist, a melanocortin agonist, a melanocortin 4 receptor agonist, a beta-3 agonist, a serotonin receptor 2C agonist, an orexin antagonist, a melanin concentrating hormone 1 antagonist, a melanin concentrating hormone 2 agonist, a melanin concentrating hormone 2 antagonist, a galanin antagonist, a CCK agonist, a CCK-A agonist, a corticotropin-releasing hormone agonist, an NPY 5 antagonist, an NPY 1 antagonist, a histamine receptor-3 modulator, a histamine receptor-3 blocker, a β-hydroxy steroid dehydrogenase-1 inhibitor, a phosphodiesterase inhibitor, a phosphodiesterase-3B inhibitor, a norepinephrine transport inhibitor, a non-selective serotonin/norepinephrine transport inhibitor, a ghrelin antagonist, a leptin derivative, a bombesin receptor subtype 3 agonist, a ciliary neurotrophic factor or a derivative thereof, a monoamine reuptake inhibitor, an uncoupling protein-1 activator, an uncoupling protein-2 activator, an uncoupling protein-3 activator, a thyroid hormone beta agonist, a fatty acid synthase inhibitor, a diacylglycerol acetyltransferase 2 inhibitor, an acetyl-CoA carboxylase-2 inhibitor, a glucocorticoid antagonist, an acyl-estrogen, a lipase inhibitor, a fatty acid transporter inhibitor, a dicarboxylate transporter inhibitor, a glucose transporter inhibitor, a sodium-glucose co-transporter, a phosphate transporter inhibitor, a serotonin reuptake inhibitor, a thiazolidinedione, Metformin, Topiramate, an opiate antagonist, a non-selective transport inhibitor, a MAO inhibitor, a glucose-dependent insulinotropic polypeptide (GIP) modulator, or an amylin receptor agonist. In some embodiments, the second therapeutic agent is a glucagon-like peptide 1 receptor agonist, a glucose-dependent insulinotropic polypeptide (GIP) modulator, or a dual glucose-dependent insulinotropic polypeptide (GIP) modulator and glucagon-like peptide 1 receptor agonist. In some embodiments, the GIP modulator is a GIP agonist. In some embodiments, the GIP modulator is a GIP antagonist. In some specific embodiments, the second therapeutic agent is selected from a group consisting of: liraglutide, semaglutide, exenatide, lixisenatide, dulaglutide, AMG133, and tirzepatide. In some embodiments, the second therapeutic agent is amylin receptor agonist. In some specific embodiments, the amylin receptor agonist is pramlintide or cagrilintide.

In some embodiments of any one of the previous methods, the subject has sarcopenic obesity.

In some embodiments of any one of the previous methods, the subject has osteosarcopenic adiposity syndrome (OSA).

In some embodiments of any one of the previous methods, the subject has weight-loss-associated loss of muscle mass. In some specific embodiments, the subject has weight-loss-associated loss of muscle mass from at least one weight-loss activity selected from: dieting, bariatric surgery, and treatment with GLP-1R agonist or SGLT2 inhibitor.

In another aspect, the present disclosure features a method of treating sarcopenic obesity in a subject in need thereof, the method includes administering CRB-913, or a salt thereof, in an amount sufficient to treat the sarcopenic obesity.

In another aspect, the present disclosure features a method of treating osteosarcopenic adiposity syndrome in a subject in need thereof, the method includes administering CRB-913, or a salt thereof, in an amount sufficient to treat the osteosarcopenic adiposity syndrome.

In some embodiments, the subject suffers from diabetes.

In some embodiments, the subject suffers from weight-loss-associated loss of muscle mass.

In some embodiments, the subject has previously been treated with a weight loss drug selected from: a glucagon-like peptide-1 (GLP-1) receptor (GLP1 R) agonist, a glucosedependent insulinotropic polypeptide (GIP) receptor (GIPR) agonist, a glucagon receptor (GCGR) agonist, and a SGLT2 inhibitor. In some specific embodiments, the weight loss drug is the GLP1 R agonist. In some further specific embodiments, the GLP1 R agonist is selected from the group consisting of: exenatide (BYETTA® and BYDUREON BCISE® (exenatide extended-release)), lixisenatide (ADLYXIN®), liraglutide (VICTOZA®, SAXENDA®), dulaglutide (TRULICITY®), semaglutide (WEGOVY®, OZEMPIC®, RYBELSUS®), tirzepatide (MOUNJARO®), survodutide, orforglipron, danuglipron, and Retatrutide (LY3437943), or a pharmaceutically acceptable salt of any of the foregoing. In other specific embodiments, the weight loss drug is the SGLT2 inhibitor. In some further specific embodiments, the SGLT2 inhibitor is selected from the group consisting of: empagliflozin, canagliflozin, dapagliflozin, and ipragliflozin, or a pharmaceutically acceptable salt of any of the foregoing.

In some embodiments, CRB-913 is administered in an amount effective to achieve weight loss in which more than 33%, 40%, 45%, or 50% (e.g., 50.1% to 100%) of the total weight lost is from fat mass. In some embodiments, the total weight lost is from fat mass is 36±3%, 40±3%, 45±3%, 50±3%, 55±3%, or 60±3%.

### DEFINITIONS

All methods described herein can be performed in any suitable order unless otherwise indicated herein or otherwise clearly contradicted by context. The use of any and all examples, or exemplary language (e.g., “such as”), is intended merely for illustration and does not pose a limitation on the scope of the present disclosure unless otherwise claimed.

The terms “a” and “an” do not denote a limitation of quantity but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

By “ascending dose regimen” or “ascending doses” is meant a regiment in which the dose administered, e.g., the average daily dose, is increased by 1.5 to 10 times incrementally over a period of time. For example, an ascending dose regimen may include a regimen in which any of the following criteria are met: (i) the average daily dose administered from the first day of treatment to at least Day 7 is less than 75% of the average daily dose administered on or after Day 28 (e.g., 70%±1%, 65±1%, 60±1%, 55±1%, or 50±1% of the average daily dose administered); or (ii) the dose administered on Day 1 is less than 75% of the dose administered on Day 28 (e.g., 70%±1%, 65±1%, 60±1%, 55±1%, or 50±1% of the average daily dose administered).

By “average daily dose” is meant the administered dose, in milligrams, of CRB-913 per unit time. The average daily dose is calculated from the instructed regimen. For example, 25 mg twice weekly is an average daily dose of 7.14 mg/day (50 mg/7 days); 10 mg weekly followed by 20 mg weekly followed by 20 mg twice weekly is an average daily dose in week 1 of 1.43 mg/day (10 mg/7 days), in week 2 of 2.86 mg/day (20 mg/7 days), and in week 3 of 5.71 mg/day (40 mg/7 days). For regimens of indefinite intervals, the average daily dose is calculated from the average of the high and low values. For example, a regimen that calls for 15 mg every seven to ten days has an average daily dose of 1.82 mg/day {(15 mg/7 days+15 mg/10 days)/2}. Average daily doses are in the range of from, for example, 1 to 25 mg/day, 1 to 50 mg/day, 5 to 50 mg/day, 1 to 100 mg/day, or 1 to 300 mg/day of CRB-913.

By “AUC_{∞}” “AUC_{0-∞}” or “Area Under the Curve_{∞}” is meant the mean integrated area under the curve for the plasma concentration of a drug, versus time from t=0 to ∞ following dosing.

As used herein, “bioavailability” refers to the fraction of drug absorbed following administration to a subject or patient under fed or fasted conditions.

The term “carrier” means a diluent, excipient, or vehicle with which a solid dispersion of CRB-913 is provided.

By “coefficient of variation” is the arithmetic standard deviation divided by the arithmetic mean for a particular pharmacokinetic parameter, where the data is obtained from a pharmacokinetic study involving 10, 12, or more subjects or patients.

By “Cₘₐₓ” is meant the mean peak concentration of a drug achieved in plasma after dosing.

By “descending dose regimen” or “descending doses” is meant a regimen in which the dose administered, e.g., the average daily dose, is decreased by at least 10% for 7 to 28 days. For example, a dose of 100 mg administered daily is reduced to 90 mg for 7 to 28 days after which time the dose is further decreased to 80 mg for 7 to 28 days.

As used herein, the term “dosage form” refers to a physically discrete unit of a compound (e.g., a CRB-913) for administration to a subject. Each unit contains a predetermined quantity of the compound. In some embodiments, such quantity is a unit dosage amount (or a whole fraction thereof) appropriate for administration in accordance with a dosing regimen that has been determined to correlate with a desired or beneficial outcome when administered to a relevant population. Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. The appropriate dosing regimen may be selected by the practicing physician based on the subject's (e.g., patient's) specific needs, e.g., tolerability, BMI, and other health conditions. In some embodiments, a given therapeutic compound, e.g., CRB-913, or formulation thereof, has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population.

By “dose administered on Day 1” is meant the sum total of all CRB-913 administered over the first 24 hours of the initial administration.

By “dosing day” is meant a day on which CRB-913 is administered to a subject, wherein the dose administered on a dosing day is the sum total of all CRB-913 administered over a 24-hour period beginning from the first administration on this day.

By “initial administration” is meant initiating administration of CRB-913 to a subject to whom a CRB-913 has not been administered in the previous 7 days. Desirably, CRB-913 has not been administered in the previous 22 days, 1 month, 2 months, or 3 months.

By “mean” is meant the arithmetic mean for a particular pharmacokinetic parameter, where the data is obtained from a pharmacokinetic study involving 10, 12, or more subjects or patients.

“Pharmaceutical compositions” are compositions including at least one active agent, such as CRB-913, and at least one other substance, such as a carrier. Pharmaceutical compositions optionally contain more than one active agent.

A “pharmaceutically acceptable excipient” means an excipient that is useful in preparing a pharmaceutical composition/combination that is generally safe, is sufficiently non-toxic, and neither biologically nor otherwise undesirable. A “pharmaceutically acceptable excipient” as used in the present application includes both one and more than one such excipient.

By “pharmaceutically acceptable polymer” is meant a polymer suitable for pharmaceutical formulation and capable of forming a solid dispersion.

As used herein, the term “solid dispersion” solid formulations including (i) amorphous CRB-913 dispersed in a polymer. In certain dosage forms of the present disclosure, the solid dispersion is a form having homogenously dispersed CRB-913 throughout the polymer in a manner that results in a single glass transition temperature T_{g}.

A “subject”, “patient”, or “host” is a human or non-human animal, including, but not limited to, simian, avian, feline, canine, bovine, equine or porcine in need of medical treatment. Medical treatment can include treatment of an existing condition, such as a disease or disorder, or a prophylactic or diagnostic treatment. In a particular embodiment, the patient or host is a human patient. In an alternative embodiment, the patient such as a host is treated to prevent a disorder or disease described herein. The term “isolated” as used herein refers to the material in substantially pure form. An isolated compound does not have another component that materially affects the properties of the compound. In particular embodiments, an isolated form is at least 60, 70, 80, 90, 95, 98 or 99% pure.

As used herein, the term “substantially amorphous,” in reference to CRB-913, refers to a sample of CRB-913 in which in which less than 20% (w/w) (e.g., less than 15%, 12%, 10%, 8%, 5%, 3%, 2%, or 1% (w/w)) is present in a crystalline form, as determined using the X-ray powder diffraction (as described in the Examples). In some embodiments, a substantially amorphous solid dosage form is a solid dosage form in which less than 2% (w/w) of CRB-913 is present in a crystalline form. In particular embodiments, a substantially amorphous solid dosage form is a solid dosage form in which there is no detectable crystalline form of CRB-913.

By “TPGS (tocopherol polyethylene glycol succinate)” is meant a compound or mixture of compounds containing one or more vitamin E moieties (e.g., a tocopherol, tocomonoenol, tocodienol, or tocotrienol) bonded to (e.g., by an ester, amide, or thioester bond) to one or more polyethylene glycol (PEG) moieties via a linker (e.g., a dicarboxylic or tricarboxylic acid). The vitamin E moiety can be any naturally occurring or synthetic form of vitamin E, including α, β, γ, and δ isoforms, and all stereoisomers of tocopherol, tocomonoenol, tocodienol, and tocotrienol. Linkers include, for example, dicarboxylic acids (e.g., succinic acid, sebacic acid, dodecanedioic acid, suberic acid, or azelaic acid, citraconic acid, methylcitraconic acid, itaconic acid, maleic acid, glutaric acid, glutaconic acid, fumaric acids, and phthalic acids). Exemplary tocopherol polyethylene glycol diesters are D-alpha-tocopheryl PEG succinate, tocopherol sebacate polyethylene glycol, tocopherol dodecanodioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol, and tocopherol phthalate polyethylene glycol. Each of the PEG moieties of the TPGS compound can be any polyethylene glycol or any PEG derivative, and can have a molecular weight of 200-6,000 kDa (e.g., 400-4,000 kDa, 500-2,000 kDa, 750-1,500 kDa, 800-1,200 kDa, 900-1,100 kDa, or 1,000 kDa). The PEG moieties can be polydisperse; that is, they can have a variety of molecular weights. PEG derivatives include, for example, methylated PEG, propylene glycol, PEG-NHS, PEG-aldehyde, PEG-SH, PEG-NH₂, PEG-CO₂H, PEG-OMe and other ethers, branched PEGs, and PEG copolymers (e.g., PEG-b-PPG-b-PEG-1100, PEG-PPG-PEG-1900, PPG-PEG-MBE-1700, and PPG-PEG-PPG-2000). Any known source of TPGS can be used in the present disclosure. An exemplary TPGS compound is tocopheryl PEG-1000 succinate (TPGS-1000), which has a PEG moiety having a molecular weight of 1,000 kDa. This TPGS is water-soluble form of natural-source vitamin E, which is prepared by esterification of crystalline D-α-tocopheryl acid succinate with polyethylene glycol 1000 (PEG 1000), and contains between 260 and 300 mg/g total tocopherol. Another exemplary TPGS compound is Water Soluble Natural Vitamin E (ZMC-USA, The Woodlands, Texas). Methods of preparing pegylated vitamin E are described in U.S. Pat. Nos. 2,680,749 and 3,102,078 and in U.S. Publication Nos. 2007/0184117 and 2007/0141203, which are herein incorporated by reference. TPGS compounds also include analogs that differ in chemical composition from tocopheryl PEG succinate (e.g., TPGS-1000) by the substitution, addition, or removal of one or more atoms, methylene (CH₂)ₙ units, or functional groups. TPGS compounds also include chromanol derivatives (e.g., 6-chromanol PEG-1000 succinate and 6-chromanol PEG-400 succinate), steroid derivatives (e.g., cholesteryl PEG-1000 succinate, cholic acid PEG-1000, dihydro cholic acid PEG-1000, litho-cholic acid PEG-1000, ursodeoxycholic acid PEG-1000, chenodeoxycholic acid PEG-1000), and others (e.g., indomethacin PEG-1000, chromone-2-carboxylic acid PEG-1000, chromone-2-carboxylic acid PEG-1100-OMe, chromone-2-carboxylic acid PEG-1500, chromone-2-carboxylic acid PEG-2000, naproxen PEG-1000, probenecid PEG-1000, 7-carboxymethoxy-4-methyl-coumarin PEG-1000, 5-(4-chlorophenyl)-2-furoic acid PEG-1000, probenecid tocopheryl PEG-1000 succinate, lithocholic acid PEG-1000, and chromone-3-carboxylic acid PEG-1000, 7-hydroxy-coumarinyl-4-acetic acid PEG-1000).

By “Tₘₐₓ” is meant the mean time after oral administration of a drug when the maximum plasma concentration of the drug or Cₘₐₓ is reached.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

The term “treatment” (also “treat” or “treating”), in its broadest sense, refers to any administration of a substance (e.g., CRB-913) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

Other features and advantages of the present disclosure will be apparent from the following detailed description, the drawings, and the claims.

### DETAILED DESCRIPTION OF THE DRAWINGS

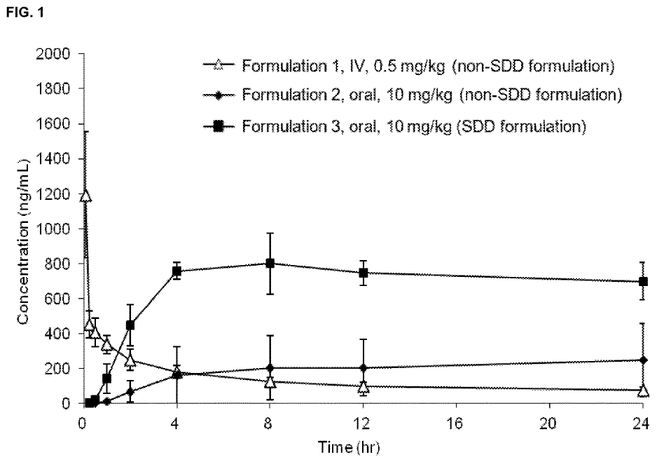

FIG. 1 is a graph showing the mean plasma concentration-time profiles of CRB-913 after an IV dose at 0.5 mg/kg (Formulation 1, see Example 5 for details), an oral dose at 10 mg/kg (Formulation 2, see Example 5 for details), an oral dose of CRB-913 in an SDD formulation (Formulation 3, see Example 5 for details) at 10 mg/kg in male Cynomolgus monkeys (n=3/time point).

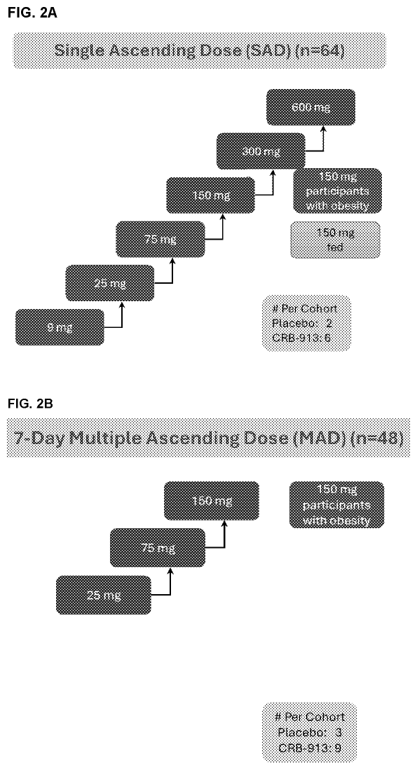

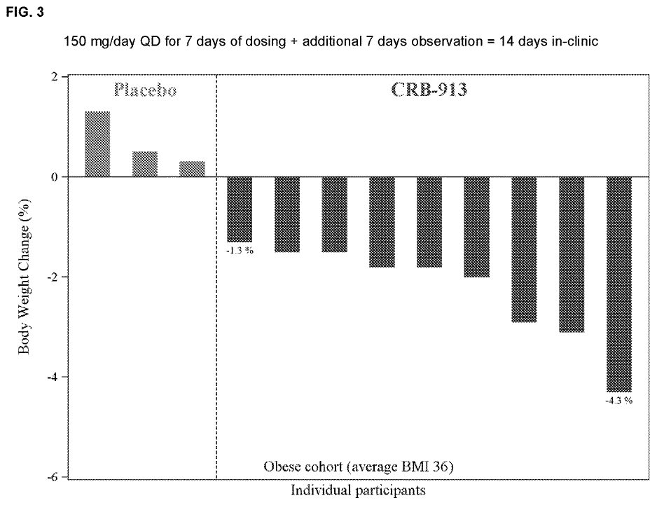

FIGS. 2A and 2B are schematics describing the dosing utilized in clinical trial phase 1a (see Example 6). FIG. 2A is a schematic describing the single ascending dose (SAD). FIG. 2B is a schematic describing the multiple ascending dose (MAD).

FIG. 3 is a bar graph of the percentage body weight change in obese subjects on day 14 after receiving a total daily dose of 150 mg for only 7 days.

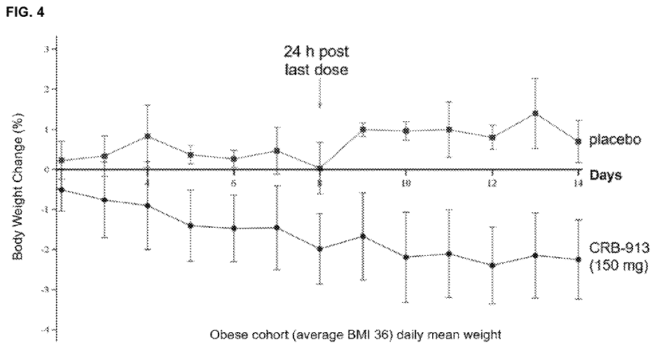

FIG. 4 contains two line graphs, one line represents the percentage body weight change in obese subjects after receiving a total daily dose of 150 mg for 7 days and the second line is the placebo results over the same time period.

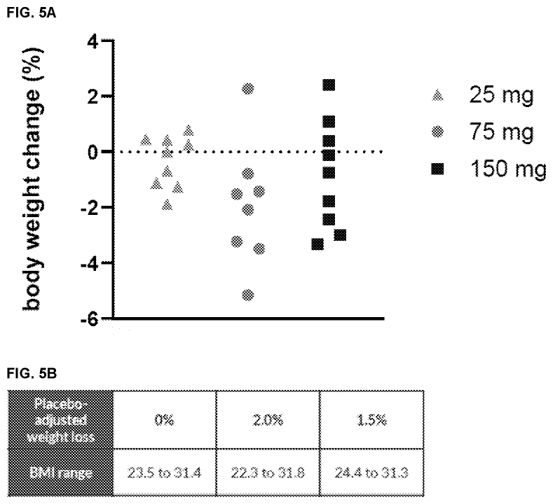

FIG. 5A is a plot of the weight change in non-obese subjects administered 25 mg, 75 mg, and 150 mg of CRB-913. FIG. 5B is a table summarizing the placebo adjusted weight loss at different BMI ranges (BMI ranges are kg/m²). The data shows a lower potential for weight loss was observed in subjects with an average BMI of 28 kg/m².

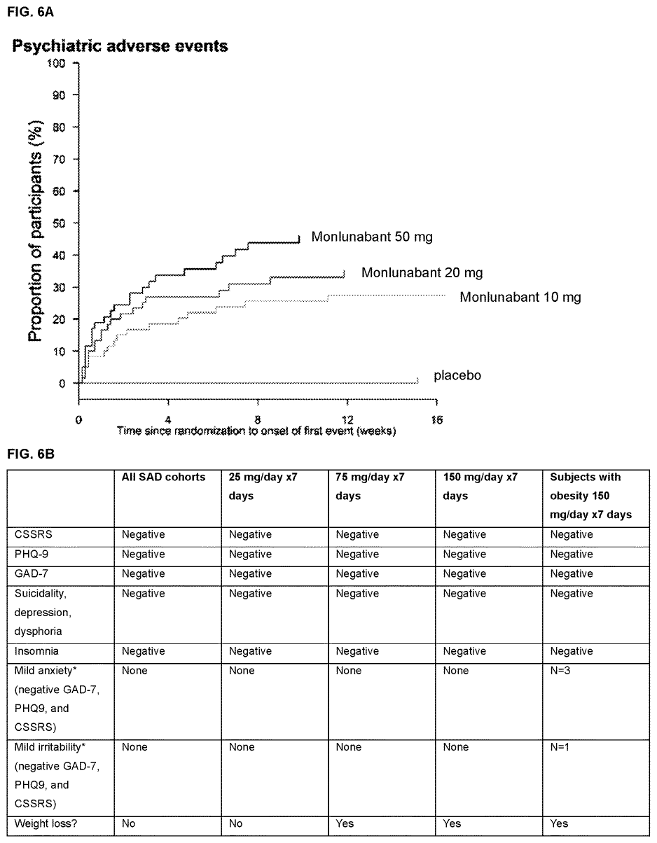

FIG. 6A is a graph depicting the psychiatric adverse events for 50 mg, 20 mg, and 10 mg doses of Monlunabant observed during Phase 2a clinical trial relative to the placebo. The trial included 240 participants. FIG. 6B is a table summarizing the CRB-913 daily neuropsychic assessments results, which are negative at all timepoints.

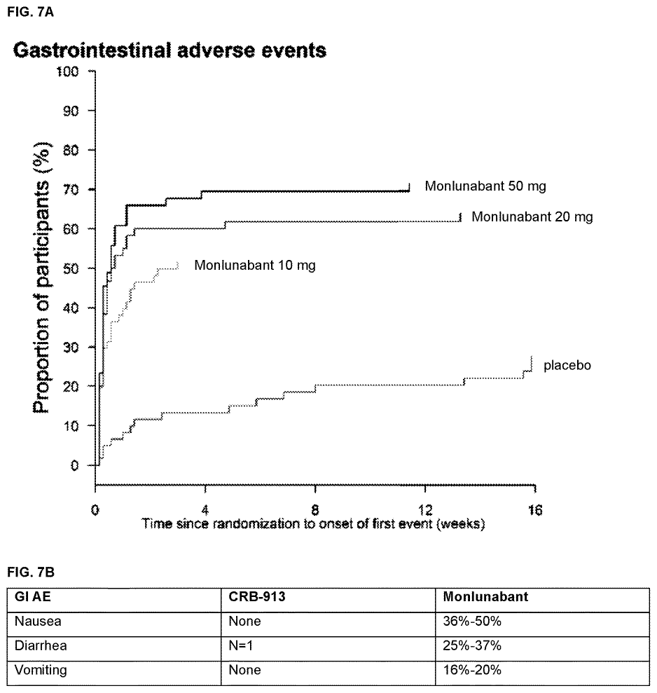

FIG. 7A is a graph depicting the gastrointestinal (GI) adverse events for 50 mg, 20 mg, and 10 mg doses of Monlunabant observed during Phase 2a clinical trial relative to the placebo. The trial included 240 participants. FIG. 7B is a table summarizing the comparative analysis between the Monlunabant results in FIG. 7A and the results from CRB-913 studies.

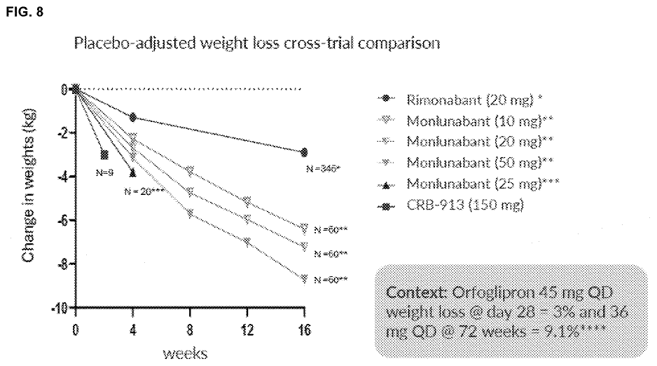

FIG. 8 is an overlay of line graphs depicting the placebo adjusted weight loss cross-trial comparison of the efficacy of CRB-913, Rimonabant, and Monlunabant. The Rimonabant and Monlunabant comparative data is taken from: doi: 10.1001/jama.295.7.761*; doi: 10.1111/dom.15353**; doi: 10.1016/S2213-8587 (25) 00216-5***; and doi: 10.1056/NEJMoa2511774****.

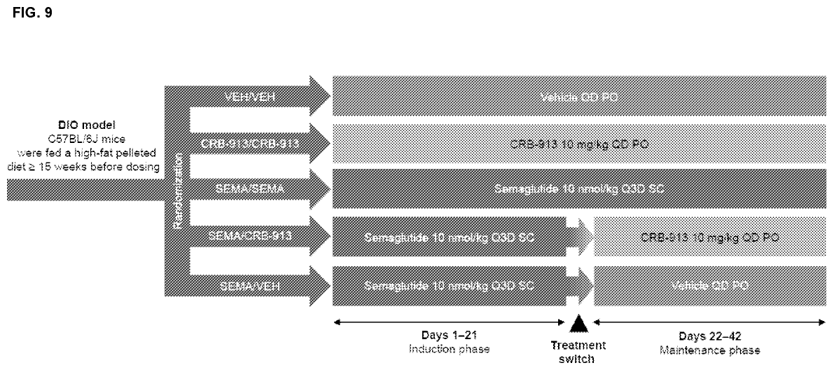

FIG. 9 is a schematic of the study design described in Example 10.

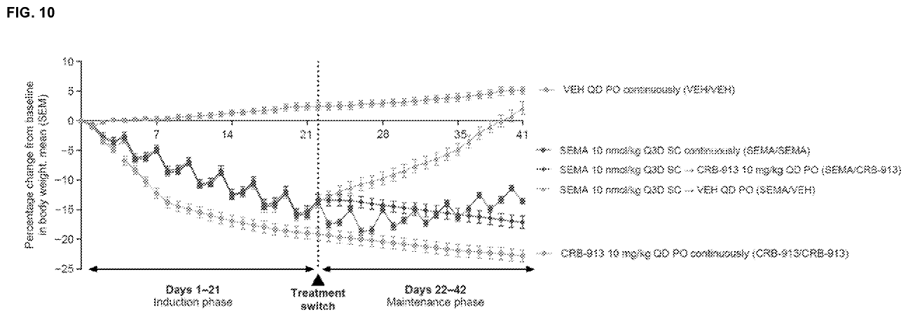

FIG. 10 is a line graph depicting percentage change from baseline to day 41 in body weight. Data is presented as mean±SEM from 10 mice/group. Statistical comparisons were conducted using ANOVA. Statistically significant changes in body weight were observed between the continuous vehicle group (VEH/VEH) and the CRB-913/CRB-913 group from days 3-42 (p<0.001), the SEMA/SEMA group at day 4 (p<0.05) and from days 6-42 (p<0.001), the SEMA/CRB-913 group from days 4-5 (p<0.05) and 6-42 (p<0.001), and the SEMA/VEH group at day 4 (p<0.01), day 5 (p<0.05) and from days 6-42 (p<0.001). Statistically significant changes in body weight were also observed between the SEMA/SEMA group and the SEMA/CRB-913 group from days 24-25 (p<0.05), days 27-28 (p<0.01) and at day 41 (p <0.01). ANOVA, analysis of variance; PO, orally; Q3D, every 3 days; QD, every day; SC, subcutaneously; SEM, standard error of the mean; SEMA, semaglutide; VEH, vehicle.

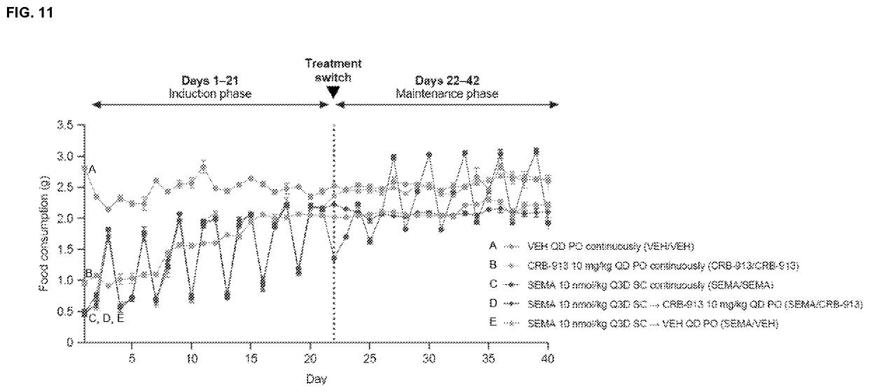

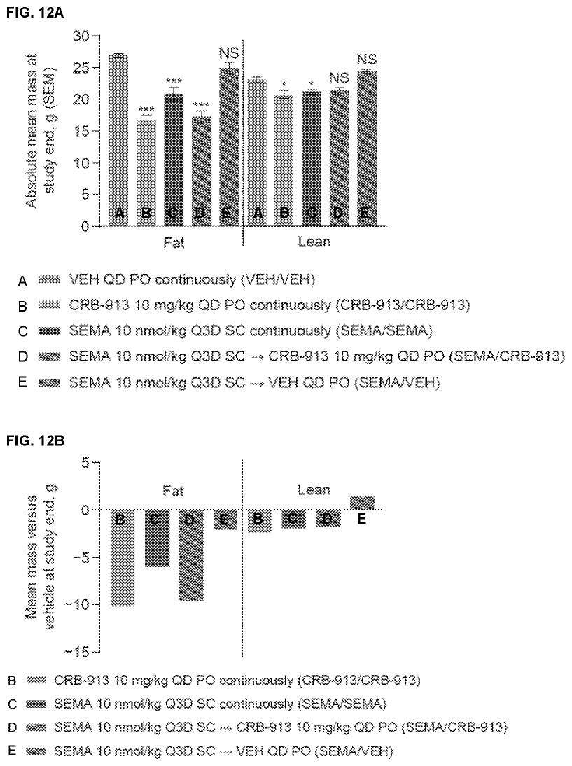

FIG. 11 is a line graph depicting percentage change from baseline to day 40 in food consumption. Data is presented as mean±SEM from 10 mice/group. Statistical comparisons were conducted using ANOVA. Statistically significant changes in food consumption were observed between the VEH/VEH group and: the CRB-913/CRB-913 group from days 1-34 and 36-40 (p >0.001 for all) and at day 35 (p<0.05); the SEMA/SEMA group from days 1-19, 21-31, 33-34, 37, and 39-40 (p<0.001 for all) and at day 36 (p<0.01); the SEMA/CRB-913 group from days 1-19 and 21-40 (p<0.001 for all) and at day 20 (p<0.05); and the SEMA/VEH group from days 1-6 and 8-19 (p<0.001 for all) and at day 7 and day 20 (p<0.01).

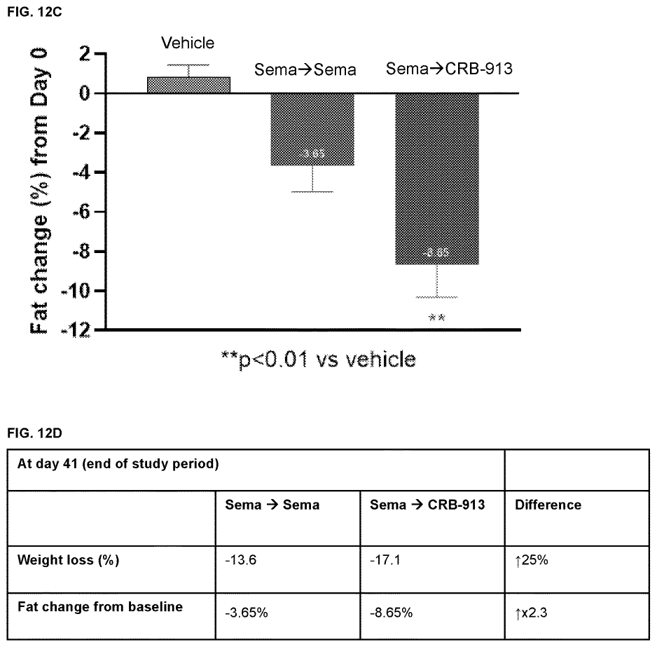

FIGS. 12A and 12B are graphs summarizing the end of study body composition. FIG. 12A shows absolute mean fat and lean mass at end of study. FIG. 12B shows Treatment effect vs. VEH/VEH in mean fat and lean body mass at end of study. Data are presented as mean±SEM from 10 mice/group. Statistical comparisons were conducted using ANOVA; * p<0.05 vs. VEH/VEH, ** p<0.01 vs. VEH/VEH, *** p<0.001 vs. VEH/VEH.

FIGS. 12C and 12D show the weight loss from CRB-913 is driven by more fat loss than semaglutide. FIG. 12C is a bar graph of the fat change from day 0 as a percentage for the vehicle, SEMA/SEMA, and SEMA/CRB-913 groups. FIG. 12D is a chart summarizing the corresponding data, which demonstrates the weight loss was from more fat loss when CRB-913 was utilized.

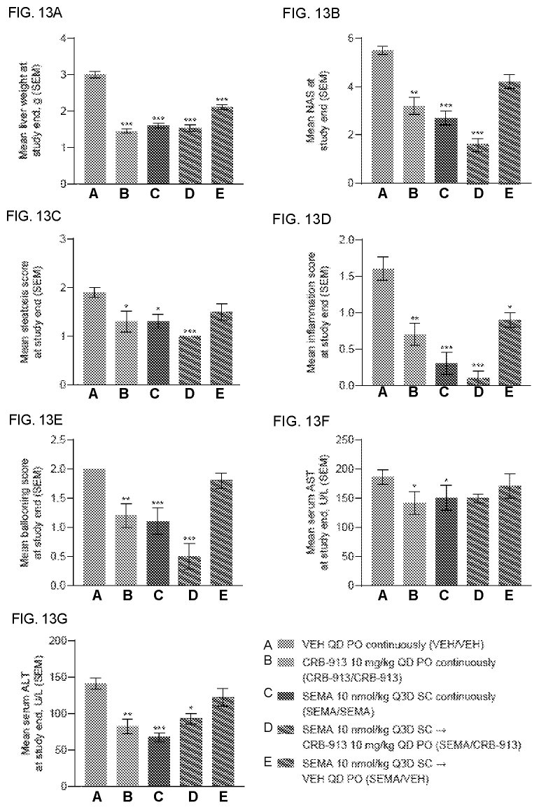

FIGS. 13A-13G are graphs summarizing the end of study liver profiling. FIG. 13A shows liver organ weight. FIG. 13B shows mean NAS. FIG. 13C shows mean steatosis score. FIG. 13D shows mean inflammation score. FIG. 13E shows mean ballooning score. FIG. 13F shows mean serum AST concentration. FIG. 13G shows mean serum ALT concentration. Data are presented as mean±SEM from 10 mice/group. Statistical comparisons were conducted using ANOVA; * p<0.05 vs. VEH/VEH, ** p<0.01 vs. VEH/VEH, *** p<0.001 vs. VEH/VEH. ALT, alanine aminotransferase; ANOVA, analysis of variance; AST, aspartate aminotransferase; H&E, hematoxylin and eosin; NAS, nonalcoholic fatty liver disease activity score; PO, orally; Q3D, every 3 days; QD, every day; SC, subcutaneously; SEM, standard error of the mean; SEMA, semaglutide; VEH, vehicle.

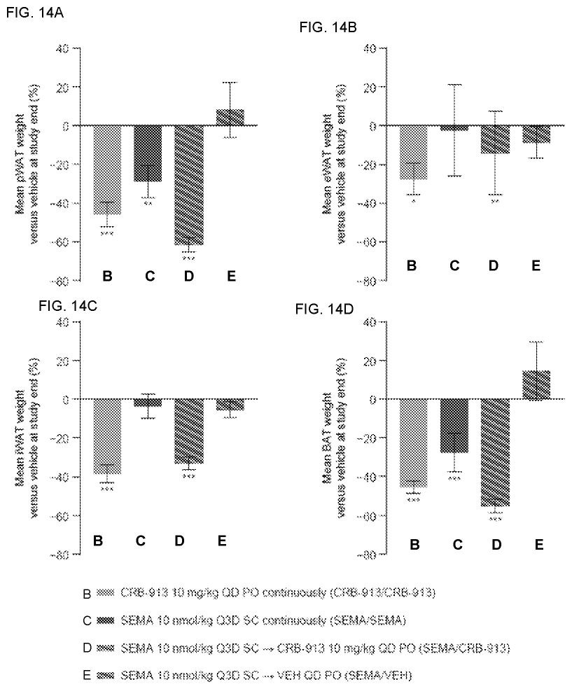

FIGS. 14A-14D are graphs summarizing the end of study body fat tissue deposits. Treatment effect vs. VEH/VEH in mean weight of pWAT (FIG. 14A), eWAT (FIG. 14B), iWAT (FIG. 14C), and BAT (FIG. 14D). Treatment effects are expressed as percentage change relative to the VEH/VEH group. Data are presented as the mean±SEM from 10 mice/group. Mean (SEM) pWAT, eWAT, iWAT, and BAT weights in the VEH/VEH group were 1521.99 (97.25), 1795.26 (137.01), 2926.43 (120.78), and 315.21 (20.89) mg, respectively. Statistical comparisons were conducted using ANOVA; * p<0.05 vs. VEH/VEH,** p<0.01 vs. VEH/VEH, *** p<0.001 vs. VEH/VEH. ANOVA, analysis of variance; BAT, scapular brown adipose tissue; eWAT, epididymal white adipose tissue; iWAT, inguinal white adipose tissue; PO, orally; pWAT, perirenal white adipose tissue; Q3D, every 3 days; QD, every day; SC, subcutaneously; SEM, standard error of the mean; SEMA, semaglutide; VEH, vehicle.

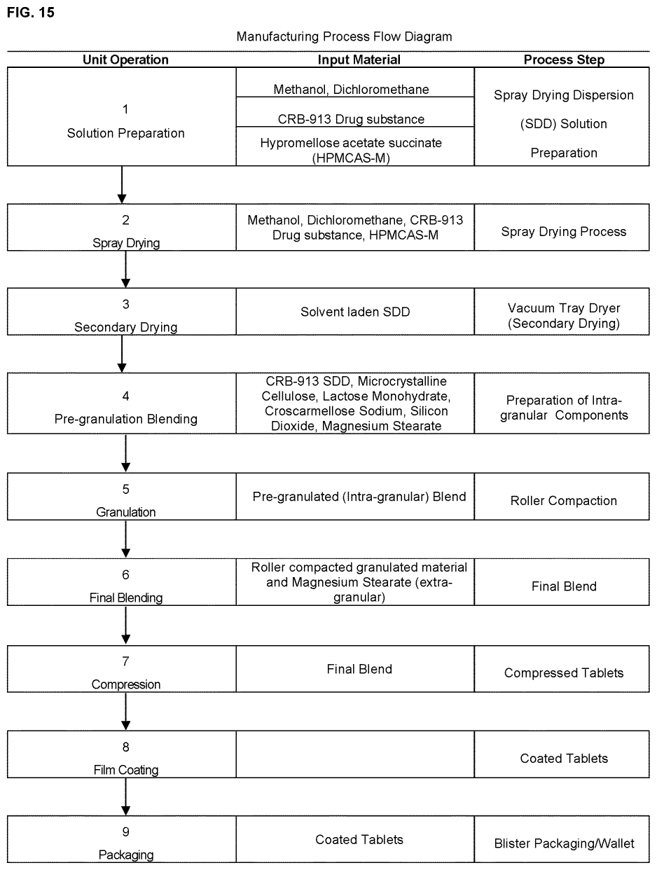

FIG. 15 is a schematic outlining the manufacturing process for the CRB-913 tablet.

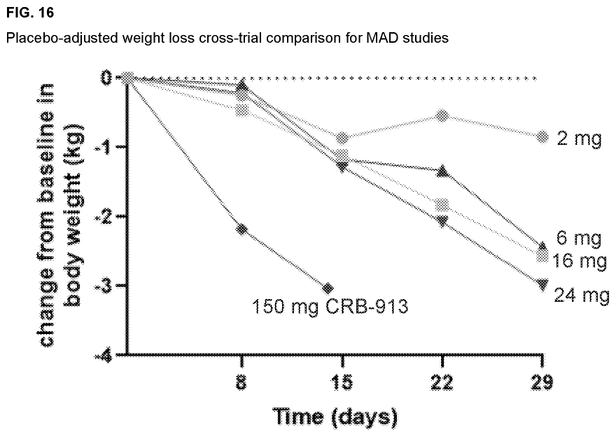

FIG. 16 is a line graph outlining the placebo-adjusted weight loss cross-trial comparison for MAD studies of CRB-913 vs. orfoglipron. The orgoglipron data is extracted from: Pratt et al. Diabetes Obes. Metab., September 2023; 25(9):2634-2641. The box data markers correspond to a 150 mg dose of CRB-913. The downward triangle data marker corresponds to a 24 mg dose of orfoglipron. The box data marker corresponds to a 16 mg dose of orfoglipron. The circle data marker corresponds to a 2 mg dose of orfoglipron.

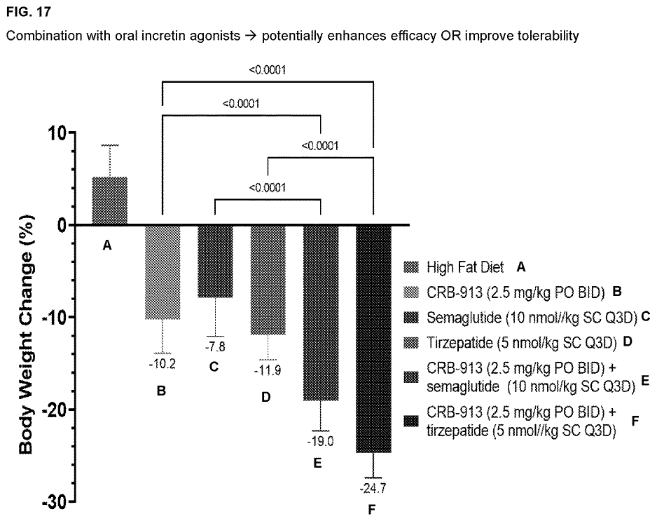

FIG. 17 is a bar graph outlining the synergistic effect achieved with combination therapies of CRB-913 and semaglutide as well as CRB-913 and tirzepatide.

### DETAILED DESCRIPTION OF THE INVENTION

The present disclosure provides formulations of CRB-913 and methods involving administration of said formulations to treat weight conditions such as obesity and co-morbidities related to obesity.

CRB-913 is a CB1 receptor inverse agonist that can treat obesity and co-morbidities thereof. The formulations of the invention can provide an advantage in the stability and/or bioavailability of CRB-913.

Accordingly, the present disclosure provides formulations to increase the oral bioavailability and/or to reduce patient-to-patient variability in pharmacokinetic behavior of CRB-913. These formulations include use of a solid dispersion system with a polymer capable of maintaining CRB-913 in an amorphous form (e.g., substantially amorphous), thereby offering improved shelf stability over other formulations.

In one aspect, the present invention is directed to pharmaceutical compositions and dosage forms thereof containing CRB-913 as the active pharmaceutical ingredient (API). CRB-913 may be present in a pharmaceutical composition of the present invention in 20% w/w to 80% w/w. In specific embodiments, CRB-913 is present in the pharmaceutical composition between 45% w/w to 55 w/w % (e.g., 48% w/w to 52% w/w, 49% w/w to 51% w/w, or 50% w/w). In further specific embodiments, CRB-913 is present in 50% w/w. In some embodiments, CRB-913 is formulated as an SDD (e.g., as described herein). Particular dosage forms include tablets (e.g., as described herein).

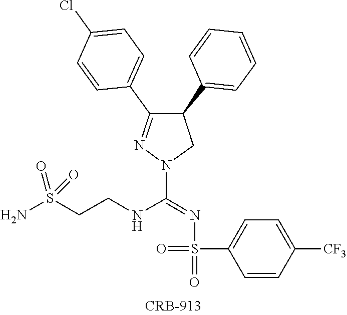 [SMILES: NS(=O)(=O)CCN/C(=N\S(=O)(=O)c1ccc(C(F)(F)F)cc1)N1C[C@H](c2ccccc2)C(c2ccc(Cl)cc2)=N1]

The synthesis, purification, and characterization of CRB-913 is described in U.S. Patent Application Publication No. US 2025-0248973, which is incorporated by reference here.

Notably, CRB-913 was able to reduce the weight of obese subjects with little to no undesired side effects. For instance, administration of CRB-913 in doses up to 600 mg did not result in significant adverse effects. Namely, CSSRS, PHQ-9, and GAD-7 scores were all low (see, e.g., Example 7). This is surprising when compared to cannabinoid receptor agonists, e.g., monlunabant, which have resulted in elevated scores in PHQ-9 and GAD-7 in some individuals (see, e.g., Example 7 and FIGS. 6A and 6B). Additionally, unlike GLP-1 agonist, CRB-913 may be administered to subjects with type 1 diabetes. As such, the use of CRB-913 for reducing weight as well as maintaining weight in subjects with diabetes, such as type 1 diabetes, could be advantageous.

### Pharmaceutical Compositions

A pharmaceutical composition of the present disclosure contains CRB-913 as the therapeutic compound. In addition to a therapeutically effective amount of the compound, the pharmaceutical compositions also contain a pharmaceutically acceptable excipient, which can be formulated by methods known to those skilled in the art. In some embodiments, the pharmaceutical compositions may be formulated and/or administered with or without other therapeutics for a particular condition. Examples of such therapeutics (second therapeutic agents) are described herein.

Exemplary routes of administration of the pharmaceutical compositions (or the compounds of the composition) include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

### Formulations for Oral Administration

The pharmaceutical compositions of the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Solid Dispersion Pharmaceutical Compositions

### Polymers for Making Solid Dispersions

Polymers (e.g., the polymeric component) for use in making the compositions disclosed herein may include one or more polymers capable of forming a solid dispersion with CRB-913. Such solid dispersion may be formed, for example, by spray dry. In some embodiments, the polymer is a pharmaceutically acceptable polymer.

Exemplary T_{g} (glass transition) values for a solid dispersion including CRB-913 and one or more polymers (e.g., the polymeric component) are from 50 °C to 125 °C (e.g., from 50 °C to 85 °C, from 50 °C to 80 °C, from 50 °C to 75 °C, from 50 °C to 70 °C, from 50 °C to 65 °C, from 50 °C to 60 °C, from 75 °C to 100 °C, from 75 °C to 95 °C, from 75 °C to 90 °C, from 75 °C to 85 °C, from 75 °C to 80 °C, from 80 °C to 110 °C, from 80 °C to 105 °C, from 80 °C to 100 °C, from 80 °C to 95 °C, from 80 °C to 90 °C, from 80 °C to 85 °C, from 85 °C to 125 °C, from 85 °C to 120 °C, from 85 °C to 115 °C, from 85 °C to 110 °C, from 85 °C to 105 °C, from 85 °C to 100 °C, from 85 °C to 95 °C, from 85 °C to 90 °C, from 90 °C to 125 °C, from 90 °C to 120 °C, from 90 °C to 115 °C, from 90 °C to 110 °C, from 90 °C to 105 °C, from 90 °C to 100 °C, from 90 °C to 95 °C, from 95 °C to 125 °C, from 95 °C to 120 °C, from 95 °C to 115 °C, from 95 °C to 110 °C, from 95 °C to 105 °C, or from 95 °C to 100 °C).

Examples of polymers (e.g., the polymeric component) which can be used in the solid dispersions of the present disclosure are, without limitation, cellulose derivatives, polyacrylates, polyvinyl pyrrolidones, polyvinyl acetates, polyethers (e.g., PEG), and copolymers thereof. These components are further described below.

### a) Cellulose Derivatives

In some embodiments, the polymeric component of the solid dispersions of the present disclosure can include one or more cellulose derivatives. Cellulose derivatives generally include those having any number of modifications to the free hydroxyl groups in cellulose.

In some examples, the cellulose derivative is a cellulose acetate having from 10% to 50% acetyl. Referring to cellulose derivatives, % refers to the proportion of the free hydroxyl groups esterified with a functional group. For example, “10% acetyl” refers to a derivative having 10% of the free hydroxyl groups in cellulose esterified with an acetyl group.

Particular examples of cellulose acetates are cellulose acetate phthalates (CAP), such as those having 35% phthalyl, 24% acetyl (available as Cellacefate); methylcellulose acetate phthalates; hydroxypropylmethyl cellulose acetates; and hydroxypropylmethyl cellulose acetate succinates (HPMCAS), such as M grade having 9% acetyl/11% succinoyl (e.g., HPMCAS having a mean particle size of 5 μm (i.e., HPMCAS-MF, fine powder grade) or having a mean particle size of 1 mm (i.e., HPMCAS-MG, granular grade)), H grade having 12% acetyl/6% succinoyl (e.g., HPMCAS having a mean particle size of 5 μm (i.e., HPMCAS-HF, fine powder grade) or having a mean particle size of 1 mm (i.e., HPMCAS-HG, granular grade)), and L grade having 8% acetyl/15% succinoyl (e.g., HPMCAS having a mean particle size of 5 μm (i.e., HPMCAS-LF, fine powder grade) or having a mean particle size of 1 mm (i.e., HPMCAS-LG, granular grade)).

Additional exemplary cellulose derivatives are alkyl celluloses, such as methyl cellulose (METHOCEL™ A) or ethylcellulose (ETHOCEL®); hydroxyalkyl celluloses, such as hydroxymethyl cellulose, hydroxyethyl cellulose, hydroxypropyl cellulose (HPC, e.g., low-substituted HPC having 11% hydroxypropyl or 8% hydroxypropyl), and hydroxybutyl cellulose; hydroxyalkylalkyl celluloses, such as hydroxyethylmethyl cellulose and hydroxypropylmethyl cellulose (hypromellose, HPMC, e.g., those having 19-24% methoxyl/7-12% hydroxypropyl (METHOCEL™ K, including those having apparent viscosity (2% in water at 20 °C) of 80-120 cP (METHOCEL™ K100), 3,000-5,600 cP (METHOCEL™ K4M), 11,250-21,000 cP (METHOCEL™ K15M), 80,000-120,000 cP (METHOCEL™ K100M)), 28-30% methoxyl/7-12% hydroxypropyl (METHOCEL™ E, including those having apparent viscosity (2% in water at 20 °C) of 3,000-5,600 cP (METHOCEL™ E4M) and 7,500-14,000 cP (METHOCEL™ E10M), also available from Dow Chemical Co.), 23% methoxyl/10% hydroxypropyl (METOLOSE® SR, available from Shin-Etsu Chemical Co., Ltd., Tokyo, Japan), 23%-29% methoxyl/8%-9% hydroxypropyl (METOLOSE®, also available from Shin-Etsu Chemical Co., Ltd.), 29% methoxyl/9% hydroxypropyl (Hypromellose USP, substitution 2910), and 23% methoxyl/6% hydroxypropyl (Hypromellose USP, substitution 2208)); hydroxyalkylalkyl cellulose esters, such as hydroxypropylmethyl cellulose phthalate (HPMCP) (e.g., HP 55 grade having 31% nominal phthalyl content and HP-55S or HP-50 grades having 24% nominal phthalyl content); carboxyalkyl celluloses, such as carboxymethyl cellulose and alkali metal salts thereof, such as sodium salts and potassium salts; carboxyalkylalkyl celluloses, such as carboxymethylethyl cellulose; and carboxyalkyl cellulose esters, such as carboxymethyl cellulose butyrate, carboxymethyl cellulose propionate, carboxymethyl cellulose acetate butyrate, and carboxymethyl cellulose acetate propionate. Any of the cellulose derivatives herein can be further cross-linked or copolymerized (e.g., with any polymer described herein).

### b) Polyacrylates

In some embodiments, the polymeric component of the solid dispersions of the present disclosure can include one or more polyacrylates or copolymers thereof. Exemplary polyacrylates are polymethacrylates; methacrylate copolymers, such as methacrylic acid-methyl methacrylate copolymers having a 1:1 ratio of free carboxyl groups to ester groups (e.g., EUDRAGIT® L 100, MW ~125,000 Da) and a 1:2 ratio of free carboxyl groups to ester groups (EUDRAGIT® S 100, MW ~125,000 Da), dimethylaminoethyl methacrylate-butyl methacrylate-methyl methacrylate copolymers (e.g., having a ratio of 2:1:1 of dimethylaminoethyl methacrylate-butyl methacrylate-methyl methacrylate and in powder, granule, or solution forms, i.e., EUDRAGIT® E PO, EUDRAGIT® E 100, or EUDRAGIT® E 12.5, respectively), and diethylaminoethyl methacrylic acid-methyl methacrylate copolymers (e.g., EUDRAGIT® E); and ethacrylate copolymers, such as methacrylic acid ethacrylate copolymers having a 50:50 ratio of methacrylic acid to ethacrylate (e.g., KOLLICOAT® MAE 100P or EUDRAGIT® L 100-55, MW ~320,000 Da).

### c) Polyvinyl Pyrrolidones and Polyvinyl Acetates

In some embodiments, the polymeric component of the solid dispersions of the present disclosure can include one or more polyvinyl pyrrolidones, polyvinyl acetates, or copolymers thereof.

Exemplary polyvinyl pyrrolidones and polyvinyl acetates are polyvinyl pyrrolidones (e.g., povidone, PVP, PVP K30, or soluble povidone) having molecular weights of 2,500 Da (KOLLIDON® 12 PF, weight-average molecular weight between 2,000 to 3,000 Da), 9,000 Da (KOLLIDON® 17 PF, weight-average molecular weight between 7,000 to 11,000 Da), 25,000 Da (KOLLIDON® 25, weight-average molecular weight between 28,000 to 34,000 Da), 50,000 Da (KOLLIDON® 30, weight-average molecular weight between 44,000 to 54,000 Da), and 1,250,000 Da (KOLLIDON® 90 or KOLLIDON® 90F, weight-average molecular weight between 1,000,000 to 1,500,000 Da); polyvinyl acetate esters, such as polyvinyl acetate phthalate (PVAP); polyethylene glycol-polyvinyl acetate copolymers, such as polyethylene glycol-polyvinylcaprolactam-polyvinylacetate copolymer (SOLUPLUS®); and polyvinyl pyrrolidone-polyvinyl acetate copolymers (PVP VA), such as those having a 60:40 ratio of N-vinyl-2-pyrrolidone to vinyl acetate (copovidone, also available as KOLLIDON® VA 64 or PVP VA64) and a 20:80 ratio of N-vinyl-2-pyrrolidone to vinyl acetate (KOLLIDON® SR).

### Methods of Making Solid Dispersion Systems

The CRB-913 in the solid dispersion may be substantially amorphous. The compositions of the present disclosure can be prepared by any useful process, such as spray drying to form a spray dried dispersion (SDD), hot melt extrusion (HME) processes, fluid bed technology, electrospinning method, or kneading method.

### Spray Dried Dispersion (SDD):

The CRB-913 SDD can be made using any useful method. Generally, one or more polymers and CRB-913, are combined either with or without a solvent (e.g., methanol, dichloromethane, or a combination thereof) to form a mixture (e.g., a liquid mixture) or a solution. Optionally, the polymer and CRB-913, either with or without additional excipients (e.g., as described herein), can be heated near or past the glass transition temperature T_{g} or melting temperature Tm to form a liquid mixture. Then, the resultant solution can be spray dried to form a solid dispersion.

In one example, one or more polymers and CRB-913 are combined with one or more solvents (e.g., dichloromethane, methanol, water, or a combination thereof) to form a solution having from 6% w/w to 18% w/w of total solids, e.g., 12% w/w. Percentage (w/w) total solids is determined by dividing the total mass of the compound (e.g., CRB-913) and one or more polymers by the total mass of the compound, one or more polymers, and one or more solvents. The solution can then be spray dried to form a SDD, which can optionally undergo further drying steps, e.g., secondary drying as described herein.

The resultant SDD can be blended with one or more excipients (e.g., as described herein) and then granulated and/or compacted to produce a final blend for encapsulating or tableting. In particular embodiments, the one or more excipients include a binding agent, a filler, a disintegrating agent, a glidant, and a lubricant.

### Other Pharmaceutically Acceptable Excipients

The solid dispersions as described herein may comprise one or more pharmaceutically acceptable excipients or may be combined with one or more pharmaceutically acceptable excipients to form a pharmaceutical composition. Pharmaceutically acceptable excipients useful in the compositions of the present disclosure can further include one or more other ingredients. In particular embodiments, the excipient is a pharmaceutically acceptable polymer, as described herein.

Exemplary excipients include anti-adherents, binders, coatings (e.g., film coating), compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Additional non-limiting exemplary excipients include colorants, flavoring agents, plasticizers, humectants, and buffering agents. In some embodiments, the excipients include one or more of lactose monohydrate (e.g., TABLETTOSE® 80), silicon dioxide (AEROSIL 200®), fumed silica (e.g., AEROSIL®), colloidal silicon dioxide (e.g., AEROSIL® 200), magnesium stearate, croscarmellose sodium, sugar alcohol (e.g., mannitol), hydroxypropyl cellulose (KLUCEL®) and microcrystalline cellulose (e.g., AVICEL® PH101 and/or AVICEL® PH102).

The pharmaceutical composition can include the solid dispersion of CRB-913 described herein in any pharmaceutically acceptable carrier. The solid dispersion may be used for filling any one of the unit dosage forms described herein (e.g., a capsule) or for tableting. The solid dispersion can optionally be further processed before filling or tableting. Exemplary further processing includes spheronizing, pelletizing, milling, injection molding, sieving, and/or calendaring the solid dispersion. In one embodiment a solid dispersion as described herein is administered to a patient in need thereof as a spray dried dispersion (SDD). In another embodiment the present disclosure provides a pharmaceutical composition comprising an SDD including CRB-913, and one or more pharmaceutically acceptable excipients as defined herein. In another embodiment any of the described spray dried dispersions can be formulated as a tablet and coated to form a coated tablet. In an alternative embodiment the spray dried dispersion is formulated into a tablet but is uncoated.

### Binders

Binders may be important to granule and tablet pharmaceutical composition of the present invention. For example, the addition of a binder as an extragranular component of a pharmaceutical composition may improve the mechanical strength of tablets disclosed herein. In some embodiments, a formulation described herein contains one binder. In other embodiments, a pharmaceutical composition described herein contains a combination of binders. Exemplary binders include starch, sugars (e.g., sucrose), natural binders (e.g., acacia), synthetic/semi-synthetic polymers (e.g., methyl cellulose or a polymer described herein above), celluloses (e.g., microcrystalline cellulose (MCC), hydroxypropyl methylcellulose (HPMC), hydroxypropyl cellulose (HPC), or a cellulose or cellulose derivative as described herein above), povidone (PVP), gelatin, polyol (e.g., mannitol or sorbitol), or polyethylene glycol (PEG). In particular embodiments of the present invention, the binder is selected from microcrystalline cellulose, hydroxypropyl cellulose, mannitol, or any combination thereof (e.g., a combination of microcrystalline cellulose and mannitol).

### Surfactants

The pharmaceutical compositions of the present disclosure may optionally include one or more surfactants. Exemplary surfactants are liquid and solid polyethoxylated esters of fatty acids, such as polyoxyl 40 stearate (MYRI® 52, hydrophobic-lipophilic balance (“HLB”)=17), PEG 400 monostearate, also known as polyoxyl 8 stearate (MYRI® 45, HLB=11), and PEG 660 hydroxystearate, also known as PEG 15 hydroxystearate (SOLUTOL® HS15, HLB=14 to 16); polyethoxylated alkyl ethers, such as polyoxyl 10 oleyl ether (BRIJ® 97, HLB=12.4) and PEG 25 cetostearyl ether (CREMOPHOR® A 25, HLB=15 to 17); polyethoxylated sorbitan esters, such as polysorbate 20 (TWEEN® 20, HLB=15) and polysorbate 80 (TWEEN® 80, HLB=11.5); polyethoxylated glyceryl esters having high HLB values (e.g., from 10 to 20), such as polyoxyl 35 castor oil (CREMOPHOR® EL, HLB=12 to 14) and polyoxyl 40 castor oil having 40-45 moles of ethylene oxide (CREMOPHOR® RH-40, HLB=14 to 16); polyethoxylated glyceryl esters of fatty acids having high HLB values (e.g., from 10 to 20), such as a mixture of PEG 6 caprylic/capric glyceryl esters having <2% C₆/50%-80% C₈/20%-50% C₁₀/<3% C₁₂/<1% C₁₄ (SOFTIGEN® 767, HLB=19), a mixture of PEG 8 caprylic/capric glyceryl esters having 50%-80% C₈/20%-50% C₁₀/<3% C₁₂/<1% C₁₈ (LABRASOL®, HLB=14), a mixture of PEG 32 lauryl glyceryl esters having 40%-50% C₁₂/14%-24% C₁₄/4%-10% C₈/3-9% C₁₀/4%-14% C₁6/5%-15% C₁₈ (GELUCIRE® 44/14, HLB=14), and a mixture of PEG 32 stearyl glyceryl esters having 40%-50% C₁₆/48%-58% C₁₈ (GELUCIRE® 50/13, HLB=13); polyethoxylated vitamin analogs, such as D-alpha-tocopheryl PEG 1000 succinate (HLB=13); and ethoxylated propoxylated block copolymers having formula H (OCH₂CH₂) a (OCHCH₃CH₂)_{b}(OCH₂CH₂)ₐOH, where a is 12 and b is 20 (POLOXAMER® 124), where a is 38 and b is 29, where a is 80 and b is 27 (POLOXAMER® 188), where a is 64 and b is 37 (POLOXAMER® 237), where a is 141 and b is 44 (POLOXAMER® 338), where a is 49 and b is 57, and where a is 101 and b is 56 (POLOXAMER® 407).

### Plasticizers

The pharmaceutical compositions of the present disclosure may optionally include one or more plasticizers. Generally, plasticizers can be used to reduce the glass transition temperature T_{g} or to decrease viscosity of a composition. Exemplary plasticizers are polyalkylene oxides, such as polyethylene glycols (e.g., PEG 300, PEG 400, PEG 4000, or PEG 8000) and polypropylene glycols; copolymers of ethylene oxide and propylene oxide, such as ethoxylated propoxylated block copolymers having the formula H (OCH₂CH₂) a (OCHCH₃CH₂)_{b}(OCH₂CH₂)ₐOH, where a is 12 and b is 20 (POLOXAMER® 124), where a is 38 and b is 29, where a is 80 and b is 27 (POLOXAMER® 188), where a is 64 and b is 37 (POLOXAMER® 237), where a is 141 and b is 44 (POLOXAMER® 338), where a is 49 and b is 57, and where a is 101 and b is 56 (POLOXAMER® 407); and polyethoxylated glyceryl esters, such as polyoxyl 35 castor oil (CREMOPHOR® EL, HLB=12 to 14) and polyoxyl 40 castor oil having 40-45 moles of ethylene oxide (CREMOPHOR® RH-40, HLB=14 to 16).

### Weight Conditions, Weight-Related Conditions, and Co-Morbidities

Subjects with a weight condition include those that are characterized as overweight or obese. A subject (e.g., a human subject) with a body mass index (BMI) of ≥30 kg/m² is considered obese. An overweight subject (e.g., a human subject) with a BMI of ≥27 kg/m² may be considered in need of weight reduction intervention if the subject has at least one other weight-related health condition, e.g., high blood pressure, high cholesterol, type 2 diabetes, sleep apnea, coronary artery disease, certain cancers, or polycystic ovary syndrome. Particularly, a subject in need of a weight reduction may include a subject with a BMI ≥27 kg/m² with a co-morbidity such as diabetes. This can include a subject with a BMI of ≥27 kg/m² and a diagnosis of type 2 diabetes according to the World Health Organization classification or other acceptable standard, with HbA1c ≥7% (≥53 mmol/mol) to ≤10% (86 mmol/mol) at Visit 1. An example of obesity includes, but is not limited to, abdominal obesity, also known as central obesity or truncal obesity, characterized as a condition where a subject (e.g., a human subject) has an excessive accumulation of fat in the abdominal region. In human subjects, abdominal obesity is defined as a waist circumference of ≥90 cm for men and ≥80 cm for women; a waist-to-hip ratio of ≥0.90 for men and ≥0.85 for women; and a BMI of ≥30 kg/m².

Subjects with a weight condition can also include subjects with weight-related conditions and co-morbidities of weight conditions. Such weight conditions, weight-related conditions, and comorbidities may include, but are not limited to, metabolic/weight issues (e.g. obesity, overweight, weight gain, binge eating disorder, delaying diabetes progress), non-metabolic/weight issues (e.g., anxiety and depression in schizophrenia), weight management, weight loss (e.g. appetite suppression, satiety), sleep apnea, diabetic nephropathy, cardiovascular disease (e.g. MACE, high blood pressure, heart failure, low HDL-C), NASH/MASH/NAFLD (e.g. fatty liver), Alzheimer's, Prader-Willi syndrome, MC4R deficiency, monogenic obesity syndromes (e.g. leptin deficiency), gastrointestinal conditions (e.g. increasing/reducing gastric motility, accelerating/delaying gastric emptying), Hepatocellular carcinoma, Prostate cancer, Pancreatic cancer, Astrocytomas, Idiopathic Pulmonary Fibrosis, substance use disorders (alcohol and nicotine) and improving blood sugar/lipids (e.g. cholesterol).

Subjects with a weight condition can also include those with sarcopenic obesity (e.g., as described herein), inflammatory myopathies, cachexia, or rapid-weight-loss-induced muscle loss.

### Sarcopenic Obesity

Declines in muscle mass, physical function, and bone density are well-recognized consequences of the natural aging process, numerous diseases such as obesity and diabetes, and the therapeutic interventions used to treat and/or manage these conditions. For example, age-related low muscle mass and/or function is defined as sarcopenia, and sarcopenic obesity describes loss of muscle and function in obese individuals (Murdock D J, Wu N, Grimsby J S, Calle R A, Donahue S, Glass D J, Sleeman M W, Sanchez R J. The prevalence of low muscle mass and/or function associated with obesity in the USA. Skelet. Muscle, December 2022; 21; 12(1):26). Older adults are vulnerable to both sarcopenia and sarcopenic obesity, driven by hormonal changes, chronic inflammation, lifestyle factors, and a higher incidence of precipitating events that contribute to reduced physical activity (Prado C M, Batsis J A, Donini L M, Gonzalez M C, Siervo M. Sarcopenic obesity in older adults: a clinical overview. Nat Rev Endocrinol., Feb. 6, 2024; doi: 10.1038/s41574-023-00943-z. Epub ahead of print. PMID: 38321142). It is estimated that 28.3% of people in the U.S. aged 60 and over have sarcopenic obesity. (Murdock D J, Wu N, Grimsby J S, Calle R A, Donahue S, Glass D J, Sleeman M W, Sanchez R J. The prevalence of low muscle mass and/or function associated with obesity in the USA. Skelet Muscle, Dec. 21, 2022; 12(1):26) Paradoxically, sarcopenic obesity can occur in the diametrically opposing metabolic scenarios of either weight gain or weight loss. In the context of weight gain, individuals can evolve in two ways: (1) they might accrue an average amount of muscle alongside the additional body weight, resulting in a condition of general obesity; and (2) if their rate of muscle accretion is low while they gain weight, they might develop sarcopenic obesity. Conversely, a similar transformation can occur in individuals experiencing considerable weight loss. The weight loss in these individuals can induce varying degrees of muscle loss, potentially leading to the development of sarcopenic obesity, especially if the loss in muscle mass and/or function is substantial. (Prado C M, Batsis J A, Donini L M, Gonzalez M C, Siervo M. Sarcopenic obesity in older adults: a clinical overview. Nat Rev Endocrinol., Feb. 6, 2024) For this reason, an increase in sarcopenic obesity is being observed in younger age groups secondary to certain weight loss treatments.

With all age groups considered, an estimated 28.7 million Americans (15.9%) are estimated to have sarcopenic obesity (Murdock D J, Wu N, Grimsby J S, Calle R A, Donahue S, Glass D J, Sleeman M W, Sanchez R J. The prevalence of low muscle mass and/or function associated with obesity in the USA. Skelet Muscle. Dec. 21, 2022; 12(1):26).

Diabetic patients are more susceptible to muscle loss compared with non-diabetic ones, potentially due to increased oxidative stress, proinflammatory state, and decreased physical activity (Ida, S. Curr. Diabetes. Rev. 2021; 17(3):293-303).

This loss of muscle mass and/or function accompanied by weight loss has been observed in diabetic patients on GLP-1R agonists (McCrimmon, R. J. et al. Diabetologia, 2020; 63(3):473-85), as well as SGLT2 inhibitors (Bolinder, J. et al. J. Clin. Endocrinol. Metab., 2012; 97(3):1020-31). The amount of fat-free mass (FFM) loss (an index of muscle mass and/or function) accounted for up to 40% of weight loss in patients taking semaglutide (Ida, S. Curr. Diabetes. Rev. 2021; 17(3):293-303). GLP-1 activation is also associated with the sympathetic nervous system and the body's stress response (Diz-Chaves, Y. et al. Nutrients, 2020; 12(11)).

One significant challenge with weight loss is weight-loss-associated loss of muscle mass. The potential health benefits of weight loss can be compromised by associated loss of muscle mass, e.g., as discussed in Nutrients, 2021; 13(7), p2473, “Weight Loss Strategies and the Risk of Skeletal Muscle Mass Loss” Mccarthy et al. Other weight loss treatments, such as bariatric surgery, can also be associated with weight-loss-associated loss of muscle mass.

In some embodiments, the subject may have sarcopenic obesity. As such, the patient may be obese and have a pre-existing loss of muscle mass and function (e.g., sarcopenia).

The invention also provides CRB-913 for use in a method of treating a subject in need thereof for sarcopenic obesity or osteosarcopenic adiposity syndrome (OSA), wherein the method includes providing to the subject an effective amount of CRB-913. As such, the treatment may prevent or reduce sarcopenia in an obese subject. OSA includes coexistence of osteopenia (or osteoporosis), sarcopenia, and excess adiposity. The method may accordingly reduce sarcopenia in a subject with OSA.

In some embodiments, the subject may also have diabetes.

In some embodiments, the subject may have weight-loss-associated loss of muscle mass. Optionally wherein the weight-loss-associated loss of muscle mass occurs from dieting, bariatric surgery, and/or treatment with GLP-1R agonist or SGLT2 inhibitor.

### Treatment Methods

In one aspect, an effective amount of CRB-913 formulated as a solid dispersion as described herein is used to treat obesity and co-morbidities of obesity.

In one embodiment, a method for the treatment of obesity is provided that includes the administration of an effective amount of a solid dispersion formulation described herein, optionally in a pharmaceutically acceptable composition. Beneficial or desired results from the method can include, but are not limited to, achieving and maintaining a healthy weight in a subject by weight loss (e.g., decreasing the body mass index (BMI) in a subject from obese to normal BMI; decreasing the waist circumference in a subject to a normal circumference; decreasing the waist-to-hip ratio in a subject to a normal ratio; or decreasing the skinfold thickness to a normal thickness in a subject).

In another embodiment, a method for the treatment of a co-morbidity of obesity is provided that includes the administration of an effective amount of a solid dispersion formulation described herein, optionally in a pharmaceutically acceptable composition. In some embodiments, the co-morbidity of obesity is diabetes, dyslipidemia, Metabolic Syndrome, dementia, a cardiovascular disease, or a hepatic disease. In some embodiments, the co-morbidity of obesity is hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; or increased incidence of malignancy of the ovaries, cervix, uterus, breasts, prostrate, or gallbladder.

In another aspect, the present invention features a method of treating a weight condition in a subject (e.g., an obese subject with a BMI of ≥30 kg/m² or a subject with a BMI of ≥27 kg/m² and at least one other weight-related health condition) wherein the method includes administering a total daily dose of 9±5 mg to 600±5 mg of CRB-913. In some embodiments, the method includes administering a total daily dose of 10±2 mg to 150±2 mg of CRB-913 (e.g., 10±2 mg, 15±2 mg, 20±2 mg, 25±2 mg, 30±2 mg, 35±2 mg, 40±2 mg, 45±2 mg, 50±2 mg, 55±2 mg, 60±2 mg, 65±2 mg, 70±2 mg, 75±2 mg, 80±2 mg, 85±2 mg, 90±2 mg, 95±2 mg, 100±2 mg, 105±2 mg, 110±2 mg, 115±2 mg, 120±2 mg, 125±2 mg, 130±2 mg, 135±2 mg, 140±2 mg, 145±2 mg, or 150±2 mg). In some particular embodiments, the subject is obesity (e.g., has a BMI of >30 kg/m²). In some specific embodiments, the CRB-913 is administered orally. In some further specific embodiments, the CRB-913 orally administered is part of a tablet formulation (e.g., as described herein).

In another aspect, the present invention features a method of reducing the body weight of a subject suffering from obesity (e.g., as described herein), wherein the method includes administering a total daily dose of 9±5 mg to 600±5 mg of CRB-913. In some embodiments, the method includes administering a total daily dose of 10±2 mg to 150±2 mg of CRB-913 (e.g., 10±2 mg, 15±2 mg, 20±2 mg, 25±2 mg, 30±2 mg, 35±2 mg, 40±2 mg, 45±2 mg, 50±2 mg, 55±2 mg, 60±2 mg, 65±2 mg, 70±2 mg, 75±2 mg, 80±2 mg, 85±2 mg, 90±2 mg, 95±2 mg, 100±2 mg, 105±2 mg, 110±2 mg, 115±2 mg, 120±2 mg, 125±2 mg, 130±2 mg, 135±2 mg, 140±2 mg, 145±2 mg, or 150±2 mg). In some specific embodiments, the CRB-913 is administered orally. In some further specific embodiments, the CRB-913 orally administered is part of a tablet formulation (e.g., as described herein).

In another aspect, the present invention features a method of maintaining a BMI <30 kg/m² in a subject previously suffering from obesity (e.g., as described herein), wherein the method includes administering a total daily dose of 9±5 mg to 600±5 mg of CRB-913. In some embodiments, the method includes administering a total daily dose of 10±2 mg to 150±2 mg of CRB-913 (e.g., 10±2 mg, 15±2 mg, 20±2 mg, 25±2 mg, 30±2 mg, 35±2 mg, 40±2 mg, 45±2 mg, 50±2 mg, 55±2 mg, 60±2 mg, 65±2 mg, 70±2 mg, 75±2 mg, 80±2 mg, 85±2 mg, 90±2 mg, 95±2 mg, 100±2 mg, 105±2 mg, 110±2 mg, 115±2 mg, 120±2 mg, 125±2 mg, 130±2 mg, 135±2 mg, 140±2 mg, 145±2 mg, or 150±2 mg). In some specific embodiments, the CRB-913 is administered orally. In some further specific embodiments, the CRB-913 orally administered is part of a tablet formulation (e.g., as described herein).

In another aspect, the present invention features a method of reducing the body weight in an overweight subject (e.g., a BMI >25 kg/m²), wherein the method includes administering a total daily dose of 9±5 mg to 600±5 mg of CRB-913. In some embodiments, the method includes administering a total daily dose of 10±2 mg to 150±2 mg of CRB-913 (e.g., 10±2 mg, 15±2 mg, 20±2 mg, 25±2 mg, 30±2 mg, 35±2 mg, 40±2 mg, 45±2 mg, 50±2 mg, 55±2 mg, 60±2 mg, 65±2 mg, 70±2 mg, 75±2 mg, 80±2 mg, 85±2 mg, 90±2 mg, 95±2 mg, 100±2 mg, 105±2 mg, 110±2 mg, 115±2 mg, 120±2 mg, 125±2 mg, 130±2 mg, 135±2 mg, 140±2 mg, 145±2 mg, or 150±2 mg). In some specific embodiments, the CRB-913 is administered orally. In some further specific embodiments, the CRB-913 orally administered is part of a tablet formulation (e.g., as described herein). In some specific embodiments, the overweight subject has a BMI of ≥27 kg/m².

In some embodiments of any of the aforementioned aspects, the subject has at least one other weight-related health condition including, but not limited to, high blood pressure, high cholesterol, diabetes, sleep apnea, coronary artery disease, certain cancers, or polycystic ovary syndrome. In some particular embodiments, the subject has diabetes. In some further particular embodiments, the subject has type 1 diabetes. In some other further particular embodiments, the subject has type 2 diabetes.

### Combination Therapy

In some embodiments, CRB-913 is administered with a second therapeutic agent, e.g., a therapeutic agent that is suitable for treating any of the disorders described herein.

Examples of second therapeutic agents suitable for administration with CRB-913 include, but are not limited to, PPAR-γ agonists (e.g., glitazones such as troglitazone, pioglitazone, englitazone, MCC-555, and rosiglitazone), biguanides (e.g., metformin and phenformin), insulin or insulin mimetics, sulfonylureas (e.g., tolbutamide or glipizide), α-glucosidase inhibitors (e.g., acarbose), HMG-CoA reductase inhibitors (e.g., lovastatin, simvastatin, pravastatin, fluvastatin, atorvastatin, or rivastatin), sequestrants (e.g., cholestyramine, colestipol, or dialkylaminoalkyl derivatives of crosslinked dextran), nicotinyl alcohol, nicotinic acid or salts thereof, PPAR-α agonists (e.g., fenofibric acid derivatives such as gemfibrozil, clofibrate, fenofibrate, and bezafibrate), inhibitors of cholesterol absorption, acyl CoA: cholesterol acyltransferase inhibitors, probucol, PPAR-α/γ agonists, ileal bile acid transporter inhibitors, insulin receptor activators, dipeptidyl peptidase IV inhibitors, exenatide, pramlintide, FBPase inhibitors, glucagon receptor antagonists, glucagon-like peptide 1, glucagon-like peptide 1 receptor agonists (e.g., orforglipron, liraglutide, semaglutide, exenatide, lixisenatide, dulaglutide, and tirzepatide), growth hormone secretagogues, growth hormone secretagogue receptor agonists, growth hormone secretagogue receptor antagonists, melanocortin agonists, melanocortin 4 receptor agonists, beta-3 agonists, serotonin receptor 2C agonists, orexin antagonists, melanin concentrating hormone 1 antagonists, melanin concentrating hormone 2 agonists, melanin concentrating hormone 2 antagonists, galanin antagonists, CCK agonists, CCK-A agonists, corticotropin-releasing hormone agonists, NPY 5 antagonists, NPY 1 antagonists, histamine receptor-3 modulators, histamine receptor-3 blockers, β-hydroxy steroid dehydrogenase-1 inhibitors, phosphodiesterase inhibitors, phosphodiesterase-3B inhibitors, norepinephrine transport inhibitors, non-selective serotonin/norepinephrine transport inhibitors (e.g., sibutramine, phentermine, or fenfluramine), ghrelin antagonists, leptin derivatives, bombesin receptor subtype 3 agonists, ciliary neurotrophic factor or derivatives thereof (e.g., axokine), monoamine reuptake inhibitors, uncoupling protein-1 activators, uncoupling protein-2 activators, uncoupling protein-3 activators, thyroid hormone beta agonists, fatty acid synthase inhibitors, diacylglycerol acetyltransferase 2 inhibitors, acetyl-CoA carboxylase-2 inhibitors, glucocorticoid antagonists, acyl-estrogens, lipase inhibitors (e.g., orlistat), fatty acid transporter inhibitors, dicarboxylate transporter inhibitors, glucose transporter inhibitors, sodium-glucose co-transporters, phosphate transporter inhibitors, serotonin reuptake inhibitors, thiazolidinediones, Metformin, Topiramate, opiate antagonists (e.g., naltrexone), non-selective transport inhibitors (e.g., bupropion), or MAO inhibitors (e.g., Moclobemide, Brofaromine, BW A616U, Ro 41-1049, RS-2232, SR 95191, Harmaline, Harman, Amiflamine, BW 1370U87, FLA 688, FLA 788; Bifemelane, Clorgyline, LY 51641, MDL 72,394, 5-(4-benzyloxyphenyl)-3-(2-cyanoethyl)-(3H)-1,3,4-oxadiazol-2-one, 5-(4-arylmethoxyphenyl)-2-(2-cyanoethyl) tetrazoles, Lazabemide, Ro 16-6491, Almoxatone, XB308, RS-1636, RS-1653, NW-1015, SL 340026, L-selegiline, Rasagiline, Pargyline, AGN 1135, MDL 72,974, MDL 72, 145, MDL 72,638, LY 54761, MD 780236, MD 240931, Bifemelane, Toloxatone, Cimoxatone, Iproniazid, Phenelzine, Nialamide, phenylhydrazine, 1-phenylcyclopropylamine, Isocarboxazid, or Tranylcypromine).

In some embodiments, CRB-913 is administered with a glucagon-like peptide 1 analog. In some embodiments, CRB-913 is administered with semaglutide. In some embodiments, CRB-913 is administered with orforglipron. In some embodiments, CRB-913 is administered with exenatide. In some embodiments, CRB-913 is administered with semaglutide.

In some embodiments, CRB-913 is administered with a glucose-dependent insulinotropic polypeptide (GIP) modulator or a dual glucose-dependent insulinotropic polypeptide (GIP) modulator a gastric inhibitory polypeptide analog. In some embodiments, CRB-913 is administered with tirzepatide.

In some embodiments, CRB-913 is administered with amylin receptor agonist.

The solid dispersion formulation, optionally in a pharmaceutically acceptable composition, as disclosed herein is also useful for administration in combination (in the same or a different dosage form) or alternation with a second pharmaceutical agent for use in ameliorating or reducing a side effect of the second pharmaceutical agent.

In further embodiments, any of the above methods of treating further includes administering to the subject a second therapeutic agent. In some embodiments, the second therapeutic agent is a PPAR-γ agonist, a biguanide, insulin or an insulin mimetic, a sulfonylurea, an α-glucosidase inhibitor, an HMG-CoA reductase inhibitor, a sequestrant, nicotinyl alcohol, nicotinic acid or a salt thereof, a PPAR-α agonist, an inhibitor of cholesterol absorption, an acyl CoA: cholesterol acyltransferase inhibitor, probucol, a PPAR-α/γ agonist, an ileal bile acid transporter inhibitor, an insulin receptor activator, a dipeptidyl peptidase IV inhibitor, exenatide, pramlintide, an FBPase inhibitor, a glucagon receptor antagonist, glucagon-like peptide 1, a glucagon-like peptide 1 receptor agonist, a growth hormone secretagogue, a growth hormone secretagogue receptor agonist, a growth hormone secretagogue receptor antagonist, a melanocortin agonist, a melanocortin 4 receptor agonist, a beta-3 agonist, a serotonin receptor 2C agonist, an orexin antagonist, a melanin concentrating hormone 1 antagonist, a melanin concentrating hormone 2 agonist, a melanin concentrating hormone 2 antagonist, a galanin antagonist, a CCK agonist, a CCK-A agonist, a corticotropin-releasing hormone agonist, an NPY 5 antagonist, an NPY 1 antagonist, a histamine receptor-3 modulator, a histamine receptor-3 blocker, a β-hydroxy steroid dehydrogenase-1 inhibitor, a phosphodiesterase inhibitor, a phosphodiesterase-3B inhibitor, a norepinephrine transport inhibitor, a non-selective serotonin/norepinephrine transport inhibitor, a ghrelin antagonist, a leptin derivative, a bombesin receptor subtype 3 agonist, a ciliary neurotrophic factor or a derivative thereof, a monoamine reuptake inhibitor, an uncoupling protein-1 activator, an uncoupling protein-2 activator, an uncoupling protein-3 activator, a thyroid hormone beta agonist, a fatty acid synthase inhibitor, a diacylglycerol acetyltransferase 2 inhibitor, an acetyl-CoA carboxylase-2 inhibitor, a glucocorticoid antagonist, an acyl-estrogen, a lipase inhibitor, a fatty acid transporter inhibitor, a dicarboxylate transporter inhibitor, a glucose transporter inhibitor, a sodium-glucose co-transporter, a phosphate transporter inhibitor, a serotonin reuptake inhibitor, a thiazolidinedione, Metformin, Topiramate, an opiate antagonist, a non-selective transport inhibitor, a MAO inhibitor, glucose-dependent insulinotropic polypeptide (GIP) modulator, or an amylin receptor agonist. In some embodiments, the second therapeutic agent is a glucagon-like peptide 1 receptor agonist (e.g., liraglutide, semaglutide, exenatide, lixisenatide, dulaglutide, or tirzepatide). In some embodiments, the second therapeutic agent is semaglutide. In some embodiments, the second therapeutic agent is tirzepatide. In other embodiments, the second therapeutic agent is a glucose-dependent insulinotropic polypeptide (GIP) modulator, either as an agonist (e.g., tirzepatide) or antagonist (e.g., AMG133). In some embodiments, the second therapeutic agent is an amylin receptor agonist (e.g., pramlintide or cagrilintide).

### Dosage Form and Dosing Regimen

In general, the present disclosure features methods of treating weight conditions, such as obesity, in a subject in need thereof, wherein the methods include administering (e.g., orally) a formulation (e.g., a pharmaceutical composition as described herein) that includes an effective amount of CRB-913.

In some embodiments, the pharmaceutical composition disclosed herein contains 5 mg to 800 mg of CRB-913. In some embodiments, the pharmaceutical composition contains 5 mg CRB-913. In some embodiments, the pharmaceutical composition contains 9 mg CRB-913. In some embodiments, the pharmaceutical composition contains 10 mg CRB-913. In some embodiments, the pharmaceutical composition contains 20 mg CRB-913. In some embodiments, the pharmaceutical composition contains 30 mg CRB-913. In some embodiments, the pharmaceutical composition contains 40 mg CRB-913. In some embodiments, the pharmaceutical composition contains 50 mg CRB-913. In some embodiments, the pharmaceutical composition contains 60 mg CRB-913. In some embodiments, the pharmaceutical composition contains 70 mg CRB-913. In some embodiments, the pharmaceutical composition contains 80 mg CRB-913. In some embodiments, the pharmaceutical composition contains 90 mg CRB-913. In some embodiments, the pharmaceutical composition contains 100 mg CRB-913. In some embodiments, the pharmaceutical composition contains 110 mg CRB-913. In some embodiments, the pharmaceutical composition contains 120 mg CRB-913. In some embodiments, the pharmaceutical composition contains 130 mg CRB-913. In some embodiments, the pharmaceutical composition contains 140 mg CRB-913. In some embodiments, the pharmaceutical composition contains 150 mg CRB-913. In some embodiments, the pharmaceutical composition contains 160 mg CRB-913. In some embodiments, the pharmaceutical composition contains 170 mg CRB-913. In some embodiments, the pharmaceutical composition contains 180 mg CRB-913. In some embodiments, the pharmaceutical composition contains 190 mg CRB-913. In some embodiments, the pharmaceutical composition contains 200 mg CRB-913. In some embodiments, the pharmaceutical composition contains 210 mg CRB-913. In some embodiments, the pharmaceutical composition contains 220 mg CRB-913. In some embodiments, the pharmaceutical composition contains 230 mg CRB-913. In some embodiments, the pharmaceutical composition contains 240 mg CRB-913. In some embodiments, the pharmaceutical composition contains 250 mg CRB-913. In some embodiments, the pharmaceutical composition contains 260 mg CRB-913. In some embodiments, the pharmaceutical composition contains 270 mg CRB-913. In some embodiments, the pharmaceutical composition contains 280 mg CRB-913. In some embodiments, the pharmaceutical composition contains 290 mg CRB-913. In some embodiments, the pharmaceutical composition contains 300 mg CRB-913. In some embodiments, the pharmaceutical composition contains 310 mg CRB-913. In some embodiments, the pharmaceutical composition contains 320 mg CRB-913. In some embodiments, the pharmaceutical composition contains 330 mg CRB-913. In some embodiments, the pharmaceutical composition contains 340 mg CRB-913. In some embodiments, the pharmaceutical composition contains 350 mg CRB-913. In some embodiments, the pharmaceutical composition contains 360 mg CRB-913. In some embodiments, the pharmaceutical composition contains 370 mg CRB-913. In some embodiments, the pharmaceutical composition contains 380 mg CRB-913. In some embodiments, the pharmaceutical composition contains 390 mg CRB-913. In some embodiments, the pharmaceutical composition contains 400 mg CRB-913. In some embodiments, the pharmaceutical composition contains 410 mg CRB-913. In some embodiments, the pharmaceutical composition contains 420 mg CRB-913. In some embodiments, the pharmaceutical composition contains 430 mg CRB-913. In some embodiments, the pharmaceutical composition contains 440 mg CRB-913. In some embodiments, the pharmaceutical composition contains 450 mg CRB-913. In some embodiments, the pharmaceutical composition contains 460 mg CRB-913. In some embodiments, the pharmaceutical composition contains 470 mg CRB-913. In some embodiments, the pharmaceutical composition contains 480 mg CRB-913. In some embodiments, the pharmaceutical composition contains 490 mg CRB-913. In some embodiments, the pharmaceutical composition contains 500 mg CRB-913. In some embodiments, the pharmaceutical composition contains 510 mg CRB-913. In some embodiments, the pharmaceutical composition contains 520 mg CRB-913. In some embodiments, the pharmaceutical composition contains 530 mg CRB-913. In some embodiments, the pharmaceutical composition contains 540 mg CRB-913. In some embodiments, the pharmaceutical composition contains 550 mg CRB-913. In some embodiments, the pharmaceutical composition contains 560 mg CRB-913. In some embodiments, the pharmaceutical composition contains 570 mg CRB-913. In some embodiments, the pharmaceutical composition contains 580 mg CRB-913. In some embodiments, the pharmaceutical composition contains 590 mg CRB-913. In some embodiments, the pharmaceutical composition contains 600 mg CRB-913. In some embodiments, the pharmaceutical composition contains 610 mg CRB-913. In some embodiments, the pharmaceutical composition contains 620 mg CRB-913. In some embodiments, the pharmaceutical composition contains 630 mg CRB-913. In some embodiments, the pharmaceutical composition contains 640 mg CRB-913. In some embodiments, the pharmaceutical composition contains 650 mg CRB-913. In some embodiments, the pharmaceutical composition contains 660 mg CRB-913. In some embodiments, the pharmaceutical composition contains 670 mg CRB-913. In some embodiments, the pharmaceutical composition contains 680 mg CRB-913. In some embodiments, the pharmaceutical composition contains 690 mg CRB-913. In some embodiments, the pharmaceutical composition contains 700 mg CRB-913. In some embodiments, the pharmaceutical composition contains 710 mg CRB-913. In some embodiments, the pharmaceutical composition contains 720 mg CRB-913. In some embodiments, the pharmaceutical composition contains 730 mg CRB-913. In some embodiments, the pharmaceutical composition contains 740 mg CRB-913. In some embodiments, the pharmaceutical composition contains 750 mg CRB-913. In some embodiments, the pharmaceutical composition contains 760 mg CRB-913. In some embodiments, the pharmaceutical composition contains 770 mg CRB-913. In some embodiments, the pharmaceutical composition contains 780 mg CRB-913. In some embodiments, the pharmaceutical composition contains 790 mg CRB-913. In some embodiments, the pharmaceutical composition contains 800 mg CRB-913.

In one embodiment, the pharmaceutical composition is formulated as a capsule and contains 5 mg to 800 mg (e.g., from 5 mg to 10 mg, from 9 mg to 20 mg, from 20 mg to 40 mg, from 20 mg to 50 mg, from 20 mg to 75 mg, from 20 mg to 100 mg, from 20 mg to 125 mg, from 20 mg to 150 mg, from 20 mg to 175 mg, from 20 mg to 200 mg, from 20 mg to 225 mg, from 30 mg to 40 mg, from 30 mg to 50 mg, from 30 mg to 75 mg, from 30 mg to 100 mg, from 30 mg to 125 mg, from 30 mg to 150 mg, from 30 mg to 175 mg, from 30 mg to 200 mg, from 40 mg to 50 mg, from 40 mg to 75 mg, from 40 mg to 100 mg, from 40 mg to 125 mg, from 40 mg to 150 mg, from 40 mg to 175 mg, from 40 mg to 200 mg, from 50 mg to 75 mg, from 50 mg to 100 mg, from 50 mg to 125 mg, from 50 mg to 150 mg, from 50 mg to 175 mg, from 50 mg to 200 mg, from 60 mg to 75 mg, from 60 mg to 100 mg, from 60 mg to 125 mg, from 60 mg to 150 mg, from 60 mg to 175 mg, from 60 mg to 200 mg, from 70 mg to 75 mg, from 70 mg to 100 mg, from 70 mg to 125 mg, from 70 mg to 150 mg, from 70 mg to 175 mg, from 70 mg to 200 mg, from 80 mg to 100 mg, from 80 mg to 125 mg, from 80 mg to 150 mg, from 80 mg to 175 mg, from 80 mg to 200 mg, from 90 mg to 100 mg, from 90 mg to 125 mg, from 90 mg to 150 mg, from 90 mg to 175 mg, from 90 mg to 200 mg, from 100 mg to 125 mg, from 100 mg to 150 mg, from 100 mg to 175 mg, from 100 mg to 200 mg, from 125 mg to 200 mg, from 150 mg to 200 mg, from 175 mg to 200 mg, from 200 mg to 300 mg, from 225 mg to 300 mg, from 250 mg to 300 mg, from 275 mg to 300 mg, from 300 mg to 325 mg, from 300 mg to 350 mg, from 300 mg to 375 mg, from 400 mg to 500 mg, from 425 mg to 450 mg, from 450 mg to 475 mg, from 500 mg to 600 mg, from 500 mg to 525 mg, from 500 mg to 550 mg, from 500 mg to 575 mg, from 600 mg to 700 mg, from 625 mg to 700 mg, from 650 mg to 700 mg, from 675 mg to 700 mg, from 700 mg to 800 mg, from 725 mg to 800 mg, from 750 mg to 800 mg, and from 775 mg to 800 mg) of CRB-913.

In one embodiment, the pharmaceutical composition is formulated as a tablet and contains 5 mg to 800 mg (e.g., from 5 mg to 10 mg, from 9 mg to 20 mg, from 20 mg to 40 mg, from 20 mg to 50 mg, from 20 mg to 75 mg, from 20 mg to 100 mg, from 20 mg to 125 mg, from 20 mg to 150 mg, from 20 mg to 175 mg, from 20 mg to 200 mg, from 20 mg to 225 mg, from 30 mg to 40 mg, from 30 mg to 50 mg, from 30 mg to 75 mg, from 30 mg to 100 mg, from 30 mg to 125 mg, from 30 mg to 150 mg, from 30 mg to 175 mg, from 30 mg to 200 mg, from 40 mg to 50 mg, from 40 mg to 75 mg, from 40 mg to 100 mg, from 40 mg to 125 mg, from 40 mg to 150 mg, from 40 mg to 175 mg, from 40 mg to 200 mg, from 50 mg to 75 mg, from 50 mg to 100 mg, from 50 mg to 125 mg, from 50 mg to 150 mg, from 50 mg to 175 mg, from 50 mg to 200 mg, from 60 mg to 75 mg, from 60 mg to 100 mg, from 60 mg to 125 mg, from 60 mg to 150 mg, from 60 mg to 175 mg, from 60 mg to 200 mg, from 70 mg to 75 mg, from 70 mg to 100 mg, from 70 mg to 125 mg, from 70 mg to 150 mg, from 70 mg to 175 mg, from 70 mg to 200 mg, from 80 mg to 100 mg, from 80 mg to 125 mg, from 80 mg to 150 mg, from 80 mg to 175 mg, from 80 mg to 200 mg, from 90 mg to 100 mg, from 90 mg to 125 mg, from 90 mg to 150 mg, from 90 mg to 175 mg, from 90 mg to 200 mg, from 100 mg to 125 mg, from 100 mg to 150 mg, from 100 mg to 175 mg, from 100 mg to 200 mg, from 125 mg to 200 mg, from 150 mg to 200 mg, from 175 mg to 200 mg, from 200 mg to 300 mg, from 225 mg to 300 mg, from 250 mg to 300 mg, from 275 mg to 300 mg, from 300 mg to 325 mg, from 300 mg to 350 mg, from 300 mg to 375 mg, from 400 mg to 500 mg, from 425 mg to 450 mg, from 450 mg to 475 mg, from 500 mg to 600 mg, from 500 mg to 525 mg, from 500 mg to 550 mg, from 500 mg to 575 mg, from 600 mg to 700 mg, from 625 mg to 700 mg, from 650 mg to 700 mg, from 675 mg to 700 mg, from 700 mg to 800 mg, from 725 mg to 800 mg, from 750 mg to 800 mg, and from 775 mg to 800 mg) of CRB-913.

In some particular embodiments, the administered dose of CRB-913 may include from 10±2 mg to 600±2 mg of CRB-913. As a linear dose was observed for doses from 9 mg to 150 mg, in specific embodiments, in specific embodiments, an administered dose of CRB-913 may include from 10±2 mg to 150±2 mg.

In some embodiments, the CRB-913 may be part of a SDD as described herein.

### Orally Dosage Forms

The CRB-913 dosage form, e.g., a pharmaceutical composition comprising CRB-913, may be a tablet as described herein. A tablet may include from 9±5 mg to 600±5 mg of CRB-913. In some embodiments, a tablet may include from 10±2 mg to 150±2 mg of CRB-913. In some embodiments, a tablet includes from 20±2 mg to 80±2 mg of CRB-913. In some embodiments, a tablet includes from 80±2 mg to 150±2 mg of CRB-913. In specific embodiments, a tablet of the present invention includes 20±2 mg, 30±10 mg, 40±10 mg, 50±10 mg, 60±10 mg, 70±10 mg, 80±10 mg, 100±20 mg, 120±20 mg, 140±10 mg, 150±2 mg, 300±2 mg, or 450±2 mg of CRB-913. In some embodiments, the tablet includes a CRB-913 SDD as described herein.

A tablet of the present invention may include a coating system. Exemplary coating systems include film coating, enteric coating, or gelatin coating. In particular embodiments, a tablet of the present invention includes a film coating. A film coating may include a solvent or vehicle, binder, plasticizer, colorant or opacifier, or any combination thereof. Exemplary solvents/vehicles include water, ethanol, methylene chloride, and other chlorinated hydrocarbons. Exemplary binders include hydroxypropyl methylcellulose (HPMC), polyvinyl Alcohol (PVA), and acrylic copolymers (e.g., EUDRAGIT®). Exemplary plasticizers include glycerol, polyethylene glycols (PEG), propylene glycol, triacetin, triethyl citrate (TEC), and diethyl phthalate. Exemplary colorants and opacifiers include titanium dioxide, iron oxides like ferric oxide red or yellow, black iron oxide (also known as ferrosoferric oxide, Fe₃O₄), and other dyes such as indigo carmine, tartrazine, allura red, quinoline yellow, pearlescent pigments, vegetable juice, carotenoids, and turmeric. Coating systems may be premixed for single application, e.g., OPADRY®.

### Administration

### General

The formulations described herein can be administered to achieve any amount/and or frequency to achieve to provide an effective amount of CRB-913 to a host to treat obesity or a co-morbidity of obesity, e.g., any of the disorders described herein, which achieves the desired therapeutic result. The amount and timing of the solid dispersion formulations of CRB-913 administered will be dependent on the host being treated, the instructions of the supervising medical specialist, the time course of the exposure, on the manner of administration, on the pharmacokinetic properties of CRB-913, and on the judgment of the prescribing clinician. Thus, because of host-to-host variability, the dosages given below are a guideline and the clinician can titrate doses of the compound to achieve the treatment that the clinician considers appropriate for the host. In considering the degree of treatment desired, the clinician can balance a variety of factors such as age and weight of the host, presence of preexisting disease, as well as presence of other diseases.

An effective amount of a formulation of CRB-913 as described herein can be used in an amount sufficient to (a) inhibit the cannabinoid receptor-1 (CB1R); (b) achieve weight loss in a subject to maintain a normal body mass index; (c) cause a cure of a co-morbidity of obesity. Accordingly, an effective amount of the solid dispersion formula or composition described herein will provide a sufficient amount of CRB-913 when administered to a patient to provide a clinical benefit.

The pharmaceutical composition may be formulated as any pharmaceutically useful form, e.g., a pill, a capsule (e.g., hard gelatin and soft gelatin), a tablet, or a powder. Some dosage forms, such as tablets and capsules, are subdivided into suitably sized unit doses containing appropriate quantities of the active components, e.g., an effective amount of CRB-913 to achieve the desired purpose.

The therapeutically effective dosage of the formulations described herein will be determined by the health care practitioner depending on the condition, size, and age of the patient as well as the route of delivery. In certain embodiments the pharmaceutical composition is in a dosage form that contains from 5 mg to 800 mg, or from 10 mg to 150 mg of the CRB-913. The dosage form can be administered, for example, once a day (QD), twice a day (BID), three times a day (TID), four times a day (QID), five times a day (OD), once every other day (Q2D), once every third day (Q3D), once every fourth day, once every fifth day, once every sixth day, once a week, as needed, or any dosage schedule that provides treatment of a disorder described herein.

The formulations disclosed herein may be administered orally. In some embodiments, CRB-913 is administered without food. In other embodiments, CRB-913 is administered with food.

In accordance with the presently disclosed methods, an oral dosage form for administration can be in any desired form in which the formulation is stable as a solid. The solid dispersion formulations as disclosed in the present disclosure have good pharmacokinetic and pharmacodynamics properties, for instance when administered by the oral routes.

### Tablet

A CRB-913 tablet of the present invention may be administered with food or within 0 to 300 minutes after the subject has consumed food, e.g., within 30 minutes, within 60 minutes, within 240 minutes, or within 300 minutes after food consumption. In some embodiments, a CRB-913 tablet is taken with or within a period of time after a meal, e.g., within 0 to 30 minutes, within 0 to 60 minutes, within 0 to 240 minutes, or within 0 to 300 minutes of breakfast, lunch, or dinner. In some embodiments, a CRB-913 tablet is taken with or within a period of time after at least one meal, e.g., after breakfast, after lunch, after dinner, or any combination thereof.

A tablet of the present invention, e.g., as described herein, may be administered daily, e.g., at least once a day. In some embodiments, the CRB-913 tablet is administered once a day. In some embodiments, the CRB-913 tablet is administered twice a day. In some embodiments, the CRB-913 tablet is administered three times a day.

In some embodiments, a tablet with 9±5 mg to 600±5 mg of CRB-913 is administered one to three times a day. When a tablet is administered multiple times a day (a 24-hour period), there may be between 0 to 12 hours between each administered dose, e.g., two doses of 20 mg CRB-913 at the same time, two doses of 20 mg CRB-913 6 hours apart, or three doses of 20 mg CRB-913 3 hours apart.

In some particular embodiments, a tablet with 10±2 mg to 150±2 mg of CRB-913 is administered one to three times a day.

In some other embodiments, a tablet with 9±5 mg to 600±5 mg of CRB-913 is administered one to four times a week. In some embodiments, a CRB-913 tablet is administered once a week. In some embodiments, a CRB-913 tablet is administered every other day (Q2D). In some embodiments, a CRB-913 tablet is administered every third day (Q3D).

CRB-913 may be administered until the weight of the subject has decreased to a desired point, e.g., a BMI of 18.5 to 25 kg/m², or until the weight condition in the subject is treated. In some embodiments, CRB-913 is administered to a subject until the subject is no longer obese, e.g., a BMI <30 kg/m². In some embodiments, a subject receives chronic administration of CRB-913, e.g., a subject is administered a dose to maintain a BMI <30 kg/m².

### Dosing Regimen

In some embodiments, the total daily dose administered is administered according to a dosing regimen, e.g., drug titration, tapering off method, or a combination thereof. In some embodiments, a dosing regimen, e.g., as described herein, is preferable to reduce undesired GI responses.

### I. Ascending Dose

In some embodiments, CRB-913 is administered to the subject in ascending doses.

In some embodiments, the ascending dose administration comprises:
(i) an initial administration of an average daily dose over a first period of at least 7 days (e.g., 7 to 28 days, 7 to 180 days, or 7 to 360 days); and
(ii) following step (i), administration of 1.5 to 4 times the average daily dose over a second period of at least 7 days (e.g., 7 to 28 days, 7 to 180 days, or 7 to 360 days).

In some embodiments, step (i) comprises administering to the subject during the first period an average daily dose of from 10±5 mg to 100±5 mg (e.g., from 10±5 mg to 50±5 mg, from 20±5 mg to 80±5 mg, or from 50±5 mg to 100±5 mg). In some embodiments, step (i) comprises administering to the subject during the first period an average daily dose of from 10±2 mg to 20±2 mg (e.g. 10±2 mg, 12±2 mg, 14±2 mg, 16±2 mg, 18±2 mg, or 20±2 mg) of CRB-913. In some embodiments, step (i) comprises administering to the subject during the first period an average daily dose of from 20±2 mg to 40±2 mg (e.g. 20±2 mg, 22±2 mg, 24±2 mg, 26±2 mg, 28±2 mg, 30±2 mg, 32±2 mg, 34±2 mg, 36±2 mg, 38±2 mg, or 40±2 mg) of CRB-913. In some embodiments, step (i) comprises administering to the subject during the first period an average daily dose of from 40±2 mg to 60±2 mg (e.g. 40±2 mg, 42±2 mg, 44±2 mg, 46±2 mg, 48±2 mg, 50±2 mg, 52±2 mg, 54±2 mg, 56±2 mg, 58±2 mg, or 60±2 mg) of CRB-913. In some embodiments, step (i) comprises administering to the subject during the first period an average daily dose of from 60±2 mg to 80±2 mg (e.g. 60±2 mg, 62±2 mg, 64±2 mg, 66±2 mg, 68±2 mg, 70±2 mg, 72±2 mg, 74±2 mg, 76±2 mg, 78±2 mg, or 80±2 mg) of CRB-913. In some embodiments, step (i) comprises administering to the subject during the first period an average daily dose of from 80±2 mg to 100±2 mg (e.g. 80±2 mg, 82±2 mg, 84±2 mg, 86±2 mg, 88±2 mg, 90±2 mg, 92±2 mg, 94±2 mg, 96±2 mg, 98±2 mg, or 100±2 mg) of CRB-913.

In some embodiments, step (ii) comprises administering to the subject during the second period an average daily dose of from 20±5 mg to 200±5 mg (e.g., from 20±5 mg to 80±5 mg, from 50±5 mg to 150±5 mg, from 100±5 mg to 150±5 mg, or from 100±5 mg to 200±5 mg). In some embodiments, step (ii) comprises administering to the subject during the second period an average daily dose of from 20±5 mg to 100±5 mg (e.g., 20±5 mg, 25±5 mg, 30±5 mg, 35±5 mg, 40±5 mg, 45±5 mg, 50±5 mg, 55±5 mg, 60±5 mg, 65±5 mg, 70±5 mg, 75±5 mg, 80±5 mg, 85±5 mg, 90±5 mg, 95±5 mg, or 100±5 mg) of CRB-913. In some embodiments, step (ii) comprises administering to the subject during the second period an average daily dose of from 80±5 mg to 200±5 mg (e.g., 80±5 mg, 85±5 mg, 90±5 mg, 95±5 mg, 100±5 mg, 105±5 mg, 110±5 mg, 115±5 mg, 120±5 mg, 125±5 mg, 130±5 mg, 135±5 mg, 140±5 mg, 145±5 mg, 150±5 mg, 155±5 mg, 160±5 mg, 165±5 mg, 170±5 mg, 175±5 mg, 180±5 mg, 185±5 mg, 190±5 mg, 195±5 mg, or 200±5 mg) of CRB-913.

In some embodiments, the method further comprises: (iii) following step (ii), administration of 2 to 8 times the average daily dose over a third period of at least 7 days (e.g., 7 to 28 days, 7 to 180 days, or 7 to 360 days), wherein the average daily dose over the second period is less than the average daily dose administered over a third period.

In some embodiments, step (iii) comprises administering to the subject during the third period an average daily dose of from 40±5 mg to 350±5 mg (e.g., from 40±5 mg to 120±5 mg, from 50±5 mg to 150±5 mg, from 100±5 mg to 300±5 mg, from 150±5 mg to 250±5 mg, from 150±5 mg to 350±5 mg, or from 250±5 mg to 350±5 mg). In some embodiments, step (iii) comprises administering to the subject during the third period an average daily dose of from 40±5 mg to 100±5 mg (e.g., 40±5 mg, 45±5 mg, 50±5 mg, 55±5 mg, 60±5 mg, 65±5 mg, 70±5 mg, 75±5 mg, 80±5 mg, 85±5 mg, 90±5 mg, 95±5 mg, or 100±5 mg) of CRB-913. In some embodiments, step (iii) comprises administering to the subject during the third period an average daily dose of from 140±5 mg to 200±5 mg (e.g., 140±5 mg, 145±5 mg, 150±5 mg, 155±5 mg, 160±5 mg, 165±5 mg, 170±5 mg, 175±5 mg, 180±5 mg, 185±5 mg, 190±5 mg, 195±5 mg, or 200±5 mg) of CRB-913. In some embodiments, step (iii) comprises administering to the subject during the third period an average daily dose of from 150±5 mg to 300±5 mg (e.g., 150±5 mg, 155±5 mg, 160±5 mg, 165±5 mg, 170±5 mg, 175±5 mg, 180±5 mg, 185±5 mg, 190±5 mg, 195±5 mg, 200±5 mg, 205±5 mg, 210±5 mg, 215±5 mg, 220±5 mg, 225±5 mg, 230±5 mg, 235±5 mg, 240±5 mg, 245±5 mg, 250±5 mg, 255±5 mg, 260±5 mg, 265±5 mg, 270±5 mg, 275±5 mg, 280±5 mg, 285±5 mg, 290±5 mg, or 300±5 mg) of CRB-913.

In some embodiments, the method further comprises (iv) following step (iii), administration of 4 to 10 times the average daily dose over a fourth period of at least 7 days (e.g., 7 to 28 days, 7 to 180 days, or 7 to 360 days), wherein the average daily dose over the third period is less than the average daily dose administered over a fourth period.

In some embodiments, step (iv) comprises administering to the subject during the fourth period an average daily dose of from 80±5 mg to 600±5 mg (e.g., from 80±5 mg to 150±5 mg, from 100±5 mg to 300±5 mg, from 150±5 mg to 450±5 mg, from 200±5 mg to 500±5 mg, from 250±5 mg to 450±5 mg, from 400±5 mg to 600±5 mg, or from 300±5 mg to 600±5 mg). In some embodiments, step (iv) comprises administering to the subject during the fourth period an average daily dose of from 80±5 mg to 150±5 mg (e.g., 80±5 mg, 85±5 mg, 90±5 mg, 95±5 mg, 100±5 mg, 105±5 mg, 110±5 mg, 115±5 mg, 120±5 mg, 125±5 mg, 130±5 mg, 135±5 mg, 140±5 mg, 145±5 mg, or 150±5 mg). In some embodiments, step (iv) comprises administering to the subject during the fourth period an average daily dose of from 180±5 mg to 250±5 mg (e.g., 180±5 mg, 185±5 mg, 190±5 mg, 195±5 mg, 200±5 mg, 205±5 mg, 210±5 mg, 215±5 mg, 220±5 mg, 225±5 mg, 230±5 mg, 235±5 mg, 240±5 mg, 245±5 mg, or 250±5 mg). In some embodiments, step (iv) comprises administering to the subject during the fourth period an average daily dose of from 280±5 mg to 350±5 mg (e.g., 280±5 mg, 285±5 mg, 290±5 mg, 295±5 mg, 300±5 mg, 305±5 mg, 310±5 mg, 315±5 mg, 320±5 mg, 325±5 mg, 330±5 mg, 335±5 mg, 340±5 mg, 345±5 mg, or 350±5 mg). In some embodiments, step (iv) comprises administering to the subject during the fourth period an average daily dose of from 380±5 mg to 450±5 mg (e.g., 380±5 mg, 385±5 mg, 390±5 mg, 395±5 mg, 400±5 mg, 405±5 mg, 410±5 mg, 415±5 mg, 420±5 mg, 425±5 mg, 430±5 mg, 435±5 mg, 440±5 mg, 445±5 mg, or 450±5 mg).

### II. Descending Dose

In some embodiments, a tapering off method is utilized wherein after the subject has been administered a dosing regimen of CRB-913 for a period of time, e.g., from 7 to 120 days, or until the BMI of the subject is <30 kg/m², a descending dose of CRB-913 is administered. In some embodiments, a descending dose of CRB-913 is administered until the dose reaches 0 mg. In some specific embodiments, the dose of CRB-913 administered is at least 10% less (e.g., 10% to 20% less, 10% to 50% less, or 20% to 60% less) than average daily dose. In some specific embodiments, the dose of CRB-913 administered is decreased by at least 10% (e.g., 10% to 20% less, 10% to 50% less, or 20% to 60% less) of the average daily dose for 7 to 28 days. In some specific embodiments, the dose of CRB-913 administered is decreased by at least 10% (e.g., 10% to 20% less, 10% to 50% less, or 20% to 60% less) of the average daily dose a week until the dose is 0 mg.

### EXAMPLES

The disclosure is further illustrated by the following examples, which are not to be construed as limiting this disclosure in scope or spirit to the specific procedures herein. It is to be understood that the examples are provided to illustrate certain embodiments and that no limitation to the scope of the disclosure is intended thereby.

### Example 1: CRB-913 Solvent Screening

In this experiment, various spray drying solvents were screened to determine which solvent is most suitable for developing a spray-dried dispersion (SDD) formulation of CRB-913. CRB-913 showed good solubility in the tested solvents, shown in Table 1.

TABLE 1
Approximate solubility of CRB-913 in Organic Solvents

| Solvent | Approximate solubility (mg/mL) |
|---|---|
| Methanol | 69 < S < 97 |
| Ethanol | 44 < S < 58 |
| Dichloromethane | 11 < S < 13 |
| Acetone | 107 < S < 144 |
| Ethyl Acetate | 138 < S < 208 |
| Tetrahydrofuran | 67 < S < 81 |
| Water | S < 2 |

Considering the solubility data in Table 1, the solvent properties, safety classification, and polymer solubility, a mixture of dichloromethane/methanol (70/30, v/v) was chosen as the spray drying solvent to develop SDD formulations of CRB-913. Alternatively, neat acetone or neat methanol were also considered as viable solvents to produce spray dried SDD formulations of CRB-913.

### Example 2: Determination of Drug Loading in CRB-913 SDD Formulations

Prior to screening SDD polymers, experiments were conducted to determine the drug load using Hypromellose 2910. The aim of the drug load experiments was to determine the optimal CRB-913 loading for the SDD formulation.

Four SDD powder batches were prepared to test the suitability of different drug loadings between 20%-50% CRB-913 (e.g., 20%, 30%, 40%, and 50% CRB-913) using Hypromellose 2910 as the polymer. The 20% CRB-913 SDD used for this characterization was used as reference.

A Buchi 290 spray dryer was used for the SDD manufacturing and the dichloromethane/methanol (70/30 v/v) solvent mixture was utilized.

The SDD powers obtained were evaluated by appearance, polarized light microscopy (PLM), SEM, HPLC (purity and drug load), XRPD, mDSC, TGA, DVS and water content (KF). No significant difference was detected with the different drug loads in any of the characterization tests with the exception of a higher water uptake for the 20% drug load vs. 50% drug load due to the higher presence of Hypromellose 2910. The SDD batches (20% and 50% CRB-913 drug load) characterized were all amorphous.

The kinetic solubility of the four SDD drug loadings was tested in biorelevant media (SGF and FaSSIF), to evaluate the drug release performance. CRB-913 drug substance was used as a control. No significant difference was detected with the different drug loads in any of biorelevant media used.

The SDD powders were also placed on a short-term stability program of 4-weeks at real time (25 °C/60% RH) and accelerated (40 °C/75% RH) conditions in open containers. No significant difference was detected with the different drug loads in any of the conditions tested. No degradation was observed, and no new impurities were formed. As expected in open containers, some water was absorbed by the SDD powder but never exceeding 2.5% intake from initial. All SDDs tested remained amorphous for the duration of the study.

In conclusion, there was no obvious difference in dissolution behavior between 20% to 50% drug load SDD. In addition, all SDD compositions were chemically and physically stable for at least 4 weeks at real time and accelerated conditions in open containers.

### Example 3: Polymer Screening for SDD Formulation Development

Nine polymers were screened using two methods (precipitation inhibition and fast evaporation). An appropriate polymer is required to produce an amorphous solid dispersion, leading to improved solubility in biorelevant media.

### Precipitation Inhibition Study

A 1% solution of each excipient in FaSSIF was spiked with CRB-913 in DMSO solution to achieve a nominal 2.5 mg/mL CRB-913 concentration. The solutions were stirred at 500 rpm at 37 °C. Samples were taken at each timepoint, centrifuged and the supernatant was diluted for HPLC analysis. Final pH of each solution was also recorded. The kinetic solubility results from this experiment are summarized in Table 2.

TABLE 2
Precipitation Inhibitor for Polymer Screening

| | Kinetic solubility (μg/mL) in FaSSIF | | | | |
|---|---|---|---|---|---|
| | (API target concentration 2.5 mg/mL) | | | | |
| Formulation | 15 | 30 | 60 | 120 | Final |
| Composition | min | min | min | min | pH |
| CRB-913 drug substance | 112 | 98 | 57 | 35 | 6.64 |
| PVP K30 (1% w/v) | 116 | 107 | 56 | 42 | 6.58 |
| PVP VA64 (1% w/v) | 132 | 128 | 69 | 85 | 6.60 |
| HPMC E5 (1% w/v) | 120 | 118 | 115 | 121 | 6.67 |
| HPMC AS LG (1% w/v) | 133 | 137 | 147 | 188 | 6.73 |
| HPMC AS MG (1% w/v) | 280 | 305 | 341 | 348 | 6.51 |
| HPMC AS HG (1% w/v) | 1252 | 1211 | 1334 | 1394 | 6.67 |
| EUDRAGIT ® L100 | 121 | 138 | 100 | 58 | 6.64 |
| (1% w/v) | | | | | |
| EUDRAGIT ® E100 | 531 | 555 | 588 | 335 | 4.94 |
| (1% w/v) | | | | | |
| Soluplus (1% w/v) | 859 | 1184 | 774 | 690 | 6.62 |

These results shown in Table 2 demonstrate that the Soluplus, HPMC-AS HG, HPMC-AS MG, and Eudragit E100 polymers exhibited good precipitation inhibition for CRB-913.

### Fast Evaporation Dispersions Kinetic Solubility Study

A solution of each polymer and CRB-913 at a 20% drug loading was prepared. The solutions were stored at approximately 80 °C for about 1 h under fast evaporation until a liquid free solid dispersion is achieved. A control of CRB-913 alone was treated in the same way. To determine solubility in gastric and intestinal fluids, the preparations were added to SGF fluid and CRB-913 concentrations were evaluated at 15, 30, and 60 minutes post addition. The SGF samples (solutions/suspensions) were then diluted into FaSSIF fluid and the media was sampled at 65, 90, 120, and 180 minutes to determine soluble concentrations of CRB-913. The results from the fast evaporation study are summarized in Table 3.

TABLE 3
Fast Evaporation Study

| | Kinetic solubility (μg/mL) | | | | | | | | |
|---|---|---|---|---|---|---|---|---|---|
| Formulation | SGF API target conc. 5 mg/mL | | | | SGF 2 × FaSSIF API target conc. 2.5 mg/mL | | | | Final |
| Composition | 15 min | 30 min | 60 min | pH | 65 min | 90 min | 120 min | 180 min | pH |
| Fast Evaporated | 1 | 2 | 4 | NR | 181 | 179 | 180 | 156 | 6.95 |
| CRB-913 | | | | | | | | | |
| CRB-913/PVP K30 | 50 | 50 | 59 | NR | 165 | 157 | 158 | 146 | 6.90 |
| (20%:80% w/w) | | | | | | | | | |
| CRB-913/PVP VA64 | 19 | 23 | 29 | NR | 192 | 192 | 198 | 188 | 6.91 |
| (20%:80% w/w) | | | | | | | | | |
| CRB-913/HPMC E5 | 506 | 471 | 499 | NR | 278 | 220 | 209 | 183 | 6.96 |
| (20%:80% w/w) | | | | | | | | | |
| CRB-913/HPMC-AS | ND | ND | ND | NR | 215 | 222 | 244 | 256 | 5.58 |
| LG (20%:80% w/W) | | | | | | | | | |
| CRB-913/HPMC AS | ND | ND | ND | NR | 202 | 200 | 219 | 244 | 5.93 |
| MG (20%:80% w/w) | | | | | | | | | |
| CRB-913/HPMC-AS | ND | ND | ND | NR | 76 | 117 | 138 | 143 | 6.51 |
| HG (20%:80% w/w) | | | | | | | | | |
| CRB-913/EUDRAGIT ® | ND | ND | ND | NR | 201 | 188 | 192 | 180 | 5.55 |
| L100 | | | | | | | | | |
| (20%:80% w/w) | | | | | | | | | |
| CRB-913/EUDRAGIT ® | 438 | 450 | 426 | NR | 24 | 16 | 18 | 17 | 7.28 |
| E100 | | | | | | | | | |
| (20%:80% w/w) | | | | | | | | | |
| CRB-913/Soluplus | 2 | 3 | 6 | NR | 65 | 60 | 63 | 64 | 6.92 |
| (20%:80% w/w) | | | | | | | | | |

NR = not recorded; ND = not detected

These results show good precipitation inhibition for HPMC E5, HPMC-AS LG, and HPMC-AS MG.

As a result of the polymer screening, HPMC-AS MG, HPMC E5, and EUDRAGIT® E100 were chosen for further development of SDD formulations.

To inform the selection of the best polymer for SDD formulation development, further studies were undertaken and 20% SDD powders were prepared with different grades of the following polymers of interest: HPMC-AS; HPMC E5, EUDRAGIT® E100, and SOLUPLUS® (polyvinyl caprolactam polyvinyl acetate-polyethylene glycol grafted copolymer). The resultant SDD powders were evaluated for characterization, kinetic solubility, and stability.

The SDD powders were evaluated by appearance, polarized light microscopy (PLM), SEM, HPLC (purity and drug load), XRPD, mDSC, TGA, DVS, water content (KF), and GC. No significant difference was detected with the different drug loads in any of the characterization tests.

The kinetic solubility of the three SDD formulations were tested in biorelevant media (SGF and FaSSIF) to evaluate the drug release performance. No significant difference was detected in any of biorelevant media used.

The SDD powders were also placed on a short-term stability program of 4-weeks at real time (25 °C/60% RH) and accelerated (40 °C/75% RH) conditions in open and closed containers. The EUDRAGIT® E100 SDD formulation was physically and chemically stable for at least 4 weeks (25 °C/60% RH open) but degraded at 40 °C/75% RH in open and closed containers after 2 weeks. No significant difference was detected with the HPMCAS MG SDD formulation and the HPMC E5 SDD formulation, which indicated that CRB-913 in these formulations was physically and chemically stable at all tested timepoints. Chiral analysis performed on SDD batches shows enantiomer level similar to the ingoing CRB-913 material.

Because of the degradation observed, the EUDRAGIT® E100 SDD formulation was considered less favorable, while HPMC E5 and HPMCAS MG formulations were further evaluated.

The HPMCAS MG and HPMC E5 SDD formulations at 50% drug load were prepared by spray drying. To make the SDD, CRB-913 was weighed into a 250 mL glass vial, the suitable selected solvent was added, and the mixture was stirred and ultrasonicated for about 30 minutes until a clear solution was obtained (the concentration of CRB-913 was 30 mg/mL). The resulting solution was then added to the corresponding polymer, and the mixture was stirred and ultrasonicated until clear solutions were obtained, followed by spray drying using the Buchi 290 to form SDDs.

The resultant SDD powders were evaluated for characterization, kinetic solubility, and stability exactly as it was done for the 20% drug load and no significant difference in the testing parameters were observed.

Dog PK studies comparing SDD 50% drug loading with HPMC E5 vs HPMC-AS MG as polymers were performed. Both polymers have a very similar pharmacokinetic behavior. HPMC E5 has a slightly inferior AUC and larger variability.

### Conclusions

Considering the solubility data, the solvent properties, and polymer solubility, dichloromethane: Methanol (70/30, v/v) was chosen as the spray drying solvent. HPMC-AS MG was chosen for further development. There was no significant difference in behavior between 20% to 50% drug load SDD.

### Example 4: Preclinical SDD formulations

Based on the experiments described in Examples 1-3, SDD formulations at two CRB-913 strengths, 50% w/w and 20% w/w, were characterized using the hypromellose acetate succinate polymer (HPMCAS, MG grade) to determine which formulation resulted in optimal properties.

TABLE 4
Formulation Composition of the CRB-913 SDD Feedstock Solutions

| | | 50% SDD Composition | | 20% SDD Composition | |
|---|---|---|---|---|---|
| | | Quantity | | Quantity | |
| | | per 100 g | % w/w | per 100 g | % w/w |
| Ingredient | Function | (solids) | (solids) | (solids) | (solids) |
| CRB-913 drug | API | 50.0 | 50.0 | 20.0 | 20.0 |
| substance | | | | | |
| Hypromellose acetate | SDD Polymer | 50.0 | 50.0 | 80.0 | 80.0 |
| succinate (HPMCAS) | | | | | |
| MG grade | | | | | |
| Methanol^{a} | SDD solvent | 270.0 | — | 270.0 | — |
| Dichloromethane^{a} | SDD solvent | 630.0 | — | 630.0 | — |

| | | | | |
|---|---|---|---|---|
| Total weight (solids) | 100.0 | 100.0 | 100.0 | 100.0 |

^{a}The solvents are removed by evaporation during manufacture.

The two formulations were prepared via the following method:
The SDD solvents were mixed, followed by the addition of CRB-913, and was stirred until dissolved.
The HPMCAS MG grade polymer was added to the solution, and was stirred until dissolved.
The solution was spray dried to produce an amorphous SDD.

The two formulation development batches were tested under two conditions, real time (25 °C/60% RH) and accelerated (40 °C/75% RH) for 28 days. The results showed an amorphous dispersion was achieved that was stable under both conditions for 28 days.

### Conclusions

The formulation composition was finalized based on stability studies and the vehicle compatibility studies. The 50% w/w SDD was chosen for further development due to a lower bulk that is expected to result in an easier reconstitution process for the oral suspension and to provide more flexibility for optimizing tablet drug load and balancing excipient bulk in a tablet formulation. The final formulation (with a representative batch size of 100 g) for the oral suspension (3 mg to 800 mg of CRB-913) comprising the 50% w/w CRB-913 SDD formulation is presented in Table 5.

TABLE 5
Formulation for the CRB-913 Powder for Oral Suspension,
3 mg to 800 mg (50% w/w CRB-913 SDD)

| | | 50% SDD Composition | |
|---|---|---|---|
| | | Quantity | |
| | | per 100 g | % w/w |
| Ingredient | Function | (Solids) | (solids) |
| CRB-913 drug substance | Active | 50.0 | 50.0 |
| Hypromellose acetate | SDD Polymer | 50.0 | 50.0 |
| succinate (HPMCAS) MG | | | |
| grade | | | |
| Methanol^{a} | SDD solvent | 270.0 | — |
| Dichloromethane^{a} | SDD solvent | 630.0 | — |

| | | |
|---|---|---|
| Total weight (solids) | 100.0 | 100.0 |

^{a}The solvents are removed by evaporation during manufacture.

### Example 5: Pharmacokinetics of CRB-913 in Cynomolgus Monkeys

The pharmacokinetic profiles of CRB-913 after single IV dose of 0.5 mg/kg CRB-913 (Formulation 1), oral dose of 10 mg/kg CRB-913 (Formulation 2), and oral dose of 10 mg/kg (Formulation 3, which is CRB-913 formulated as a spray dried dispersion comprising 19.5% (w/w) CRB-913 and 80.5% (w/w) HPMC E3) was determined in fasted male Cynomolgus monkeys. Formulations 1-3 are prepared as described below:

### Formulation 1

Formulation 1 is designed for IV administration and comprises 0.25 mg/mL CRB-913 in 5% DMSO, 5% Kolliphor HS15 in saline. Formulation 1 was prepared by the following method:
1) Weighed 12.46 mg of CRB-913 into a new tube.
2) Added 2.462 mL of DMSO into the tube.
3) Stirred the tube for 1 min.
4) Added 2.462 mL of Kolliphor HS15 into the tube.
5) Stirred the tube for 1 min.
6) Added 44.318 mL of saline into the tube. Stirred the tube for 1 min.

### Formulation 2

Formulation 2 is designed for oral administration and comprises 2 mg/mL CRB-913, 0.5% methylcellulose 400 cP in purified water as a homogenous suspension. Formulation 2 was prepared by the following method:
1) Weighed 184.70 mg of CRB-913 into a new tube.
2) Added 91.242 mL of 0.5% methylcellulose 400 cP in purified water into the tube.
3) Stirred the tube for 12 min and sonicated it for 22 min.
4) Homogenized the mixture with ULTRA-TURRAX T25 on medium speed for about 4 min.
5) Stirred the suspension at all times prior to dosing.

### Formulation 3

Formulation 3 is an SDD formulation comprising 19.5% CRB-913 and 80.5% HPMC E3 designed for oral administration, wherein the concentration of CRB-913 is 2 mg/mL. The SDD powder is dissolved in 0.5% HPMC in water to form a homogeneous suspension. Formulation 3 was prepared by the following method:
1) Weighed 927.56 mg of CRB-913 SDD powder into a new tube.
2) Added 90.405 mL of 0.5% HPMC in water into the tube.
3) Stirred the tube for 32 min.
4) Homogenized the mixture with ULTRA-TURRAX® T25 on medium speed for about 3 min.
5) Stirred the suspension at all times prior to dosing.

### Study Subjects and Dosing

A total of 9 male Cynomolgus monkeys, non-naive, approximately 4~6 kg of bodyweight, originally purchased from Hainan Jingang Biotech Co. LTD and with Qualification No. of SCXK (GD) 2015-0001 460012000000323 was taken from stock and placed on study. The animals were fasted overnight prior to dosing and the food was resumed at 4 hours post dose.

The IV dose was conducted via cephalic vein injection and the oral dose was conducted via gavage. The animals were restrained manually, and blood was collected from cephalic and saphenous veins into pre-cooled K2EDTA tubes. The blood sample was put on wet ice and centrifuged at 4 °C to obtain plasma within 15 minutes of sample collection. LC-MS/MS was used to measure the amount of CRB-913 from the plasma. The pharmacokinetic parameters were calculated using non-compartmental analysis using PHOENIX WINNONLIN® 8.2 software (Certara) with the method of linear trapezoidal linear interpolation.

### PK Results

After the IV dose of CRB-913 as API at 0.5 mg/kg in male Cynomolgus monkeys, concentrations of CRB-40913 in monkey plasma declined with a terminal half life (T_{1/2}) of 17.9±3.00 hours. The MRT_{INF} was 23.4±2.96 hour. The total clearance (CL) and volume of distribution at steady state (Vₛₛ) were 0.098±0.0254 L/hr/kg and 2.24±0.306 L/kg, respectively. The area under the curve from time 0 to last time point (AUCₗₐₛₜ), from time 0 to infinity (AUC_{INF}) and from 0 to 24 hr (AUC₀₋₂₄ₕᵣ) were 3361±677 hr*ng/ml, 5330±1340 hr*ng/ml and 3361±677 hr*ng/ml, respectively.

After PO dose of CRB-40913 as API at 10 mg/kg, concentrations of CRB-40913 in monkey plasma declined with a terminal half-life (T_{1/2}) of 53.4 hours. The lag-time (T_{lag}) was 0.500 hours. The Cₘₐₓ value was 256±200 ng/ml, and corresponding mean Tₘₐₓ value was 18.7±9.24 hours. The MRT_{INF} was 80 hours. The area under the curve from time 0 to last quantifiable concentration (AUCₗₐₛₜ), from time 0 to infinity (AUC_{INF}) and from 0 to 24 hr (AUC₀₋₂₄ₕᵣ) were 4513±3804 hr*ng/ml, 11527 hr*ng/ml and 4513±3804 hr*ng/ml, respectively. Based on the AUC_{INF} after the IV dose of 0.5 mg/kg and the PO dose of 10 mg/kg, the bioavailability of this compound in male Cynomolgus monkey was estimated to be 6.71±5.66%.

After PO dose of CRB-40913 in formulated SDD2 at 10 mg/kg, concentrations of CRB-40913 in monkey plasma declined with a terminal half-life (T_{1/2}) of 24.2 hours. The lag-time (T_{lag}) was 0.250 hours. The Cₘₐₓ value was 863±107 ng/ml, and corresponding mean Tₘₐₓ value was 14.7±8.33 hours. The MRT_{INF} was 38.3 hours. The area under the curve from time 0 to last quantifiable concentration (AUCₗₐₛₜ), from time 0 to infinity (AUC_{INF}) and from 0 to 24 hr (AUC₀₋₂₄ₕᵣ) were 16403±303 hr*ng/ml, 37651 hr*ng/ml and 16403±303 hr*ng/ml, respectively. Based on the AUC_{INF} after the IV dose of 0.5 mg/kg and the PO dose of 10 mg/kg, the bioavailability of this compound in male Cynomolgus monkey was estimated to be 24.4±0.450%.

Table 6 summarizes the pharmacokinetics of CRB-913 administered in Cynomolgus monkeys.

TABLE 6
Pharmacokinetic Parameters of CRB-913 after an IV Dose
of Formulation 1 at 0.5 mg/kg, an Oral Dose of Formulation
2 at 10 mg/kg, and an Oral Dose of Formulation 3 in
Male Cynomolgus Monkeys (N = 3/time point)

| | | | Formulation 3 |
|---|---|---|---|
| PK parameters | Formulation 1 | Formulation 2 | (SDD formulation, |
| (units) | (IV 0.5 mg/kg) | (Oral 10 mg/kg) | oral 10 mg/kg) |
| Rsq_adjusted | 0.837 | 0.997 | 0.882 |
| CL (L/hr/kg) | 0.0980 | | |
| Vₛₛ (L/kg) | 2.24 | | |
| Tₘₐₓ (hr) | | 18.7 | 14.7 |
| Cₘₐₓ (ng/mL) | | 256 | 863 |
| T_{1/2} (hr) | 17.9 | 53.4 | 24.2 |
| C₀ (ng/mL) | 1947 | | |
| T_{lag} (hr) | | 0.500 | 0.250 |
| Regression | NA | NA | NA |
| Points (hr) | | | |
| AUCₗₐₛₜ (hr · | 3361 | 4513 | 16403 |
| ng/mL) | | | |
| AUC_{INF} (hr · | 5330 | 11527 | 37651 |
| ng/mL) | | | |
| AUC_{0-24 hr} (hr · | 3361 | 4513 | 16403 |
| ng/mL) | | | |
| MRT_{INF} (hr) | 23.4 | 80.0 | 38.3 |
| F (%) | | 6.71% | 24.4% |

After a single oral dose of Formulation 2 or Formulation 3 administered to fasted male cynomolgus monkeys at 10 mg/kg, the plasma concentrations of CRB-913 in Formulation 3 and in Formulation 2 declined from peak with a t_{1/2} of 24.2 and 53.4 hours, respectively, though these differences are not significant given the short PK time course evaluated. The Cₘₐₓ of the Formulation 3 was more than 3-fold greater than that of Formulation 2 (863 and 256 ng/ml, respectively) with a Tₘₐₓ of 14.7 and 18.7 hours, respectively. Additionally, Formulation 3 resulted in >3-fold higher AUCₗₐₛₜ and AUC_{inf} (16,400 and 37,600 h·ng/mL, respectively) compared to Formulation 2 (4510 and 11,500 h·ng/ml, respectively). After a single IV dose of CRB-913 at 0.5 mg/kg (Formulation 1), the AUC_{inf} was 5330 h·ng/ml. Based on the AUC_{inf} after oral and IV dosing at 10 and 0.5 mg/kg, respectively, the bioavailability of both formulations of CRB-913 was low. However, Formulation 3 exhibited approximately 3.6-fold higher (24.4%) bioavailability compared to the Formulation 2 (6.71%). The results in Table 6 demonstrate that the SDD formulation improved the pharmacokinetic profile and bioavailability of CRB-913 in monkeys compared to non-SDD formulations.

The mean plasma concentration time profiles of Formulations 1-3 were also analyzed in this study. As shown in FIG. 1, Formulation 3 exhibited superior mean concentration of CRB-913 after 24 hours of administration compared to Formulations 1 and 2. At the 24 hour timepoint, Formulation 3 exhibited 9-fold higher plasma concentration of CRB-913 compared to Formulation 1, and 3-fold higher plasma concentration of CRB-913 compared to Formulation 2. Thus, the SDD formulation (Formulation 3) demonstrated a superior pharmacokinetic profile of CRB-913 compared to non-SDD formulations in Cynomolgus monkeys.

### Example 6: Pre-Clinical Studies of CRB-913 Therapy

The nonclinical toxicology program of CRB-913 was evaluated in pivotal GLP toxicology studies of 4 weeks and 13 weeks duration in male and female CD-1 mice and beagle dogs.

Oral administration of CRB-913 doses up to 1000 mg/kg/day to mice once daily (QD) for up to 90 days was well tolerated and without adverse changes. The no-observed-adverse-effect-level NOAEL in the 13-week study was 1000 mg/kg, which was associated with exposures that represent a >13-fold safety margin relative to the expected exposures at steady state.

The NOAEL dose in dogs was 10 mg/kg/day, which was associated with exposures that represent a >7.6-fold safety margin relative to the expected exposures at steady state.

### Example 7: Phase 1a Clinical Studies of CRB-913 Therapy

The clinical study was a 2-part Phase 1 study. The objective of this study was to assess the safety, tolerability, and PK of single (Part 1) and multiple (Part 2) ascending doses of CRB-913 administered as an oral suspension to healthy, and overweight or obese but otherwise healthy (Part 2 only), male and female subjects.

Part 1 was a single ascending dose (SAD) study design. It enrolled 7 cohorts each containing 8 subjects. Within each cohort, subjects were randomized in a ratio of 6 active: 2 placebo. A single dose was administered ranging from 9 mg to 600 mg to each participant in each cohort. Two additional cohorts looked at food effects with normal and high BMI at 150 mg single dose administration. The doses administered were 9 mg, 25 mg, 75 mg, 150 mg, 300 mg and 600 mg in healthy fasted subjects (Table 7A). Food effects were tested in the 150 mg dose cohort with healthy subjects and a second cohort of overweight or obese subjects. For Cohorts 1 to 6, subjects had a BMI in the range of 18.0 kg/m² to 32.0 kg/m²; for Cohort 7, subjects had a BMI of >32.0 kg/m².

Part 2 was a multiple ascending dose (MAD) study design. It enrolled 4 cohorts each containing 12 subjects. Within each cohort, subjects were randomized in a ratio of 9 active: 3 placebo. Doses were administered once daily for seven (7) days, where appropriate, to each cohort. Each cohort received a dose ranging from 25 mg to 150 mg without food prior to dosing. A fourth cohort at 150 mg tested CRB-913 with food in a cohort of high BMI participants. The MAD doses were 25 mg, 75 mg and 150 mg (Table 7B).

The study plan for Part 1 and Part 2 is outlined in FIGS. 2A and 2B and the following table outlines the dosing regimen for each cohort (Table 7A and Table 7B).

TABLE 7A
SAD Dosing Regimen

| Cohort | Regimen | Investigational Medicinal Product | Dose | | Route of Administration |
|---|---|---|---|---|---|
| 1 | A | CRB-913 or matching placebo | 9 | mg | Oral, Fasted |
| 2 | B | CRB-913 or matching placebo | 25 | mg | Oral, Fasted |
| 3 | C | CRB-913 or matching placebo | 75 | mg | Oral, Fasted |
| 4 | D | CRB-913 or matching placebo | 150 | mg | Oral, Fasted |
| 4 | E | CRB-913 or matching placebo | 150 | mg | Oral, Fed |
| 5 | F | CRB-913 or matching placebo | 300 | mg | Oral, Fasted |
| 6 | G | CRB-913 or matching placebo | 600 | mg | Oral, Fasted |
| 7 | H | CRB-913 or matching placebo | 150 | mg | Oral, Fed |
| (high BMI | | | | | |
| cohort) | | | | | |

Regimens administered on a single occasion (Day 1).

TABLE 7B
MAD Dosing Regimen

| Cohort | Regimen | Investigational Medicinal Product | Dose | | Route of Administration |
|---|---|---|---|---|---|
| 1 | A | CRB-913 or matching placebo | 25 | mg | Oral, Fasted |
| 2 | B | CRB-913 or matching placebo | 75 | mg | Oral, Fasted |
| 3 | C | CRB-913 or matching placebo | 150 | mg | Oral, Fasted or Oral, Fed |
| 4 | D | CRB-913 or matching placebo | 150 | mg | Oral, Fasted or Oral, Fed |
| (high BMI | | | | | |
| cohort) | | | | | |

Regimens administered once daily for 7 consecutive days (Day 1 to 7).

### Phase 1a Formulation

CRB-913 is a small molecule formulated for oral delivery. CRB-913 powder for oral suspension was compounded in unit dose strengths of 9 mg to 600 mg. The CRB-913 powder for oral suspension was composed of a spray-dried dispersion (SDD) containing 50% w/w CRB-913 drug substance and hypromellose acetate succinate.

The CRB-913 powder for oral suspension (all strengths) was suspended in 60 mL of ORA-BLEND®, a citrus berry flavored oral suspending vehicle, at the clinic to produce the final CRB-913 oral suspension.

The spray-dried formulation was designed to increase drug solubility and has been shown to enhance bioavailability of the drug in animal models and decrease variability of exposure.

### Study Design Part 1

Each cohort followed the same study sequence. Subjects underwent preliminary screening procedures for the study at the screening visit (Day-28 to Day-2). Subjects were admitted to the clinical unit in the morning on the day before dosing (Day-1) and administered a single dose of IMP at the appropriate dose for each cohort. Subjects remained on site until discharge from the clinical unit at 168 h post-dose (Day 8).

Additionally, subjects in Cohort 4 returned to the clinical unit on the morning of Period 2 Day-1, following a washout of 5 half-lives of the IMP, and remained on site until discharge from the clinical unit at 168 h post-dose (Day 8). On the day before fasted dosing, subjects were fasted overnight for a minimum of 10 h until the following morning (Day 1), when they were administered investigational medicinal product (IMP) in the fasted state. On the day before fed dosing (planned to be Regimens E and H), subjects were fast from all food and drink (except water) for a minimum of 10 h until the following morning, when they were provided with a standard high-fat breakfast; IMP administered approximately 30 min after the high-fat breakfast. For each regimen, subjects were given a LISTERINE® strip immediately prior to dosing to mask the taste of the suspension and maintain the blind. Blood samples were collected at regular intervals for safety and PK assessments from Day 1 until discharge from the clinical unit. A follow-up visit was taken place 3 to 7 days post-discharge (Days 11 to 15; Period 2 only for Cohort 4) to ensure the ongoing wellbeing of the subjects.

Subjects were asked to self-report AEs up to 30 days post-final dose of study treatment, at which point a final follow-up call was taken on Day 31, during which each subject underwent Columbia-Suicide Severity Rating Scale (C-SSRS), Patient Health Questionnaire (PHQ-9) and General Anxiety Disorder Questionnaire (GAD-7) assessments conducted by an investigator or nurse trained in using the C-SSRS, PHQ-9 and GAD-7.

### Study Design Part 2

Each cohort followed the same study sequence. Subjects underwent preliminary screening procedures for the study at the screening visit (Day-28 to Day-2). Subjects were admitted to the clinical unit in the morning on the day before dosing (Day-1) and remained on site until discharge from the clinical unit at 168 h post-final dose (Day 14). Subjects were administered IMP once daily in the morning of Days 1 to 7, where appropriate; subjects were fasted overnight for a minimum of 10 h and were administered IMP in the fasted state. For the fed cohorts IMP was administered approximately 30 min after a high-fat breakfast on Days 1 and 7 and a standard breakfast on Days 2 to 6. Subjects were given a Listerine strip immediately prior to dosing to mask the taste of the suspension and maintain the blind.

Blood samples were collected at regular intervals for safety and PK assessments from Day 1 until discharge from the clinical unit. A follow-up visit took place 3 to 7 days post-discharge (Days 17 to 21) to ensure the ongoing wellbeing of the subjects. Subjects were asked to self-report AEs up to 30 days post-final dose of study treatment, at which point a final follow-up call was taken place (Day 37), during which each subject underwent C-SSRS, PHQ-9 and GAD-7 assessments conducted by an investigator or nurse trained in using the C-SSRS, PHQ-9 and GAD-7.

### Safety Data

CRB-913 was administered to healthy volunteers in the fasted state as SAD ranging from 9 mg to 600 mg, a fed state single dose at 150 mg, and MAD ranging from 25 mg to 150 mg daily for 7 days. All single doses were well tolerated with no treatment-related AEs reported at any dose. Multiple doses up to 150 mg QD for 7 days were event-free and well tolerated. AEs deemed related to study intervention were infrequent and included mild headache and epigastric pain. There were no clinically significant changes in electrocardiogram (ECG), physical examinations, or laboratory assessments.

### Clinical Experience

CRB-913 was evaluated in a Phase 1a single ascending dose (SAD)/multiple ascending dose (MAD) study (see FIGS. 2A and 2B). For the Phase 1a studies, CRB-913 oral powder for reconstitution was utilized. Results are summarized below.

Dose range and Safety Data

CRB-913 was administered to healthy volunteers in the fasted state as SAD ranging from 9 mg to 600 mg including a fed state single dose cohort at 150 mg. CRB-913 was additionally administered to healthy volunteers with a high BMI in the fasted state at 150 mg. CRB-913 was administered to healthy volunteers in the MAD ranging from 25 mg to 150 mg daily (e.g., 25 mg/day, 75 mg/day, and 150 mg/day) for 7 days, where appropriate, including healthy volunteers with a high BMI in the fasted state at 150 mg. All single doses were well tolerated with no treatment-related AEs reported at any dose. Multiple doses up to 150 mg QD for 7 days, where appropriate, were event-free and well tolerated. See FIGS. 3, 4, 5A, and 5B.

### Pharmacokinetics and Metabolism

The PK in humans was characterized by dose increases in exposure (maximum plasma concentration [Cₘₐₓ], area under the plasma concentration-time curve from time 0 to the time of the last measurable (positive) concentration [AUC₀₋ₗₐₛₜ], and area under the plasma concentration-time curve from time 0 to infinity [AUC_{0-inf}]) after single (9 mg to 600 mg) and multiple doses (25 to 150 mg/day) in normal BMI participants under fasted conditions. After a single dose in normal BMI participants, CRB-913 was absorbed with a time to maximum plasma concentration (Tₘₐₓ) of approximately 4 hours post-dose in the fasted state and 5 hours in the fed state; in high BMI participants, Tₘₐₓ occurred at 6 hours in the fed state. Exposure was higher in the fed state compared with the fasted state, resulting in a 1.3-fold increase in Cₘₐₓ and 1.6-fold increase in AUC₀₋ₗₐₛₜ.

### Risk Assessment

A Phase 1a study demonstrated that CRB-913 was well tolerated in healthy volunteers at single doses from 9 mg to 600 mg and of up to 7 daily doses from 25 mg/day to 150 mg/day. No clinically significant adverse events (AEs) were reported in healthy (non-obese) volunteers. Loss of appetite, an anticipated effect of CRB-913, was reported by 3 participants. Vital signs were generally typical of healthy volunteer populations and normal variability was seen across placebo and actively treated groups. The degree of subclinical/undiagnosed hypertension was most evident in the obese population as expected and, again, also present in placebo treated subjects. The summative assessments using PHQ9, GAD-7, and C-SSRS were administered every day that the patients were on the Phase 1a unit and no measurement ever went above the threshold for normality (Table 8). This is notable when compared to the psychiatric adverse events observed for Monlunabant during phase 2a clinical trial, which saw >60% of AEs in the first 14 days, >95% AEs in the first 60 days, 180 patients on the treatment experienced 111 events, cases of depression where noted in phase 1a and phase 2a, and the psychiatric AEs were the most common cause of dropout in the study (FIGS. 6A and 6B).

TABLE 8
CRB-913 Daily Neuropsychic Assessments
Negative at All Timepoints

| | | | | | Subjects |
|---|---|---|---|---|---|
| | | | | | with |
| | | | | | obesity |
| | All | 25 mg/ | 75 mg/ | 150 mg/ | 150 mg/ |
| | SAD | day × 7 | day × 7 | day × 7 | day × 7 |
| | cohorts | days | days | days | days |
| CSSRS | Negative | Negative | Negative | Negative | Negative |
| PHQ-9 | Negative | Negative | Negative | Negative | Negative |
| GAD-7 | Negative | Negative | Negative | Negative | Negative |
| Suicidality, | Negative | Negative | Negative | Negative | Negative |
| depression, | | | | | |
| dysphoria | | | | | |
| Insomnia | Negative | Negative | Negative | Negative | Negative |
| Mild | None | None | None | None | N = 3 |
| anxiety* | | | | | |
| (negative | | | | | |
| GAD-7, | | | | | |
| PHQ9, and | | | | | |
| CSSRS) | | | | | |
| Mild | None | None | None | None | N = 1 |
| irritability* | | | | | |
| (negative | | | | | |
| GAD-7, | | | | | |
| PHQ9, and | | | | | |
| CSSRS) | | | | | |
| Weight loss? | No | No | Yes | Yes | Yes |

*Transient and fully resolved without any intervention

### GI Tolerability Profile

CRB-913 demonstrated a highly favorable GI tolerability profile. This is notable considering the GI AE rates observed during the Phase 2a clinical trials for Monlunabant, which saw >90% of GI AEs in the first 14 days, GI AE rates of 51-79% depending on dose, and the GI adverse events were the second most common cause of drop out in the study (FIGS. 7A and 7B; Table 9)

TABLE 9
CRB-913 Phase 1a GI Tolerability Profile (n = 112)

| GI AE | CRB-913 | Monlunabant |
|---|---|---|
| Nausea | None | 36%-50% |
| Diarrhea | N = 1 | 25%-37% |
| Vomiting | None | 16%-20% |

### Weight Loss

Weight loss with CRB-913 was observed to start early and deepen (FIG. 4). Compared to similar treatments, Rimonabant and Monlunabant, the 150 mg dose of CRB-913 demonstrated notable efficacy (FIG. 8). However, a lower potential for weight loss was observed in the non-obese cohort (FIGS. 5A and 5B). The MAD data from the CRB-913 studies suggest that, compared to orfoglipron, the placebo-adjusted weight loss for CRB-913 potentially provides a deeper and faster weight loss (FIG. 16). Table A summarizes the weight loss in 2 weeks across oral MAD obesity clinical data sets.

TABLE A
Weight Loss in 2 Weeks Across Oral
MAD Obesity Clinical Data Sets^{#}

| Drug | Placebo Adjusted WL (%) | Type |
|---|---|---|
| CRB-913 (150 mg) | −2.9% | Small molecule |
| Orforglipron (2 mg) | −1.4% | Small molecule |
| Aleniglipron (5 mg) | −1.3% | Small molecule |
| Elecoglipron (50 mg) | 0% | Small molecule |
| Semaglutide (40 mg) | −0.7% | Peptide |
| VK2735 (30 mg) | −1.8% | Peptide |

^{#}data points extracted from Pratt et al. Diabetes Obes. Metab., September 2023; 25(9): 2634-2641; Haggag et al. Diabetes Obes. Metab., February 2025; 27(2): 551-562; Davies et al. JAMA., October 2017; 17; 318(15): 1460-1470; Viking therapeutics corporate presentation, February 2026; and ACCESS Phase 2b Structure Therapeutics Press Release, November 2024.

Conclusions from the CRB-913 SAD/MAD Data
CRB-913 elicits weight loss that starts early and deepens
effect driven by best-in-class peripheral restriction
weight loss not driven by GIAEs
high peripheral restriction=favorable safety and tolerability

### Example 8: Phase 1b Clinical Study Design for CRB-913 Therapy

A 2-part, Phase 1b clinical study designed to evaluate the safety, PK, and efficacy of CRB-913 in participants with obesity will be performed. Part 1 is open label and Part 2 is double blind.

### Part 1

Part 1 is designed to measure the PK parameters of a tableted formulation of CRB-913. The study is a single dose with PK measurements over 3 days post dose with a 28-day follow-up period. The dose to be tested in Part 1 is 80 mg, which has been shown to be well tolerated.

Following a maximum 28-day screening period, 12 healthy male and female volunteers will receive a single dose of CRB-913 administered in a 20-mg tablet formulation at a total dose of 80 mg (4 tablets).

The volunteers will have assessments on Days 1 and 2 and then discharged on Day 3. Participants will return on Day 28 for follow-up assessments.

### Part 2

Part 2 will randomize approximately 240 participants, with a BMI ≥30 kg/m² but otherwise healthy males and females. The aim of Part 2 is to determine the efficacy of CRB-913 in weight loss in participants with obesity of BMI ≥30 kg/m², safety, and PK. There will be a 28-day screening period, a 12-week treatment period, and a 28-day follow-up period.

After the 28-day screening period, study participants will be randomized in a 1:1:1:1 ratio to one of 4 dose arms: matched placebo, 20, 40, and 60 mg CRB-913. Study participants assigned to 40 mg CRB-913 will be started at 20 mg/day for 2 weeks and then escalated to 40 mg/day on Day 15; those assigned to 60 mg will be started at 20 mg/day for 2 weeks, escalated to 40 mg/day starting on Day 15 and escalated to 60 mg/day on Day 29 through the remainder of the dosing period. CRB-913 will be taken QD with food for 12 weeks followed by a 28-day post-dose evaluation period. The objectives will be to assess the safety, tolerability, and PK of CRB-913 taken QD for 12 weeks and to assess the efficacy of CRB-913 with respect to body weight change versus placebo.

### Scientific Rationale for Study Design

### a) Study Population Rationale

This study will enroll male and female adult participants with obesity defined as having BMI ≥30 kg/m². As CRB-913 is a therapeutic designed to induce weight loss, this represents a patient population that can potentially benefit from CRB-913 treatment.

### b) Dose Rational

Part 1 is designed to evaluate relative bioavailability of the tablet formulation compared to data previously obtained with the dose suspension formulation (e.g., as described herein). The dose of 80 mg in Part 1 was selected to provide a dose comparable to the 75 mg dose evaluated previously (e.g., as described herein). The 75 mg dose was safe and well tolerated.

The doses selected for Part 2, 20 mg/day, 40 mg/day, and 60 mg/day, have been demonstrated to be safe and well tolerated in healthy volunteers for up to 7 days. These doses are predicted to result in a range of steady state exposures (AUC₀₋ₜₐᵤ) that were associated with significant weight loss (from 8.9% to 22%) in a murine model of diet induced obesity. The doses will result in graded increases in exposure, which will allow the evaluation of dose-related effects on safety and efficacy endpoints. The protocol includes a within-patient dose-escalation to potentially allow habituation to the effects of the study intervention and further improve tolerability at the higher doses. Neuropsychiatric AEs can show habituation/accommodation just as much as they can show accumulation or sensitization, and this graduated lead-in was considered the most likely approach minimize occurrence of untoward AEs.

The study interventions that will be used in this part of the clinical study are presented in Table 10.

TABLE 10
Study Interventions

| Study Intervention | CRB-913 Tablets for oral | Placebo for CRB-913 Tablets |
|---|---|---|
| Name: | administration | for oral administration |
| Product Type: | Active | Placebo |
| Dosage Formulation: | Round Tablet | Round Tablet |
| Unit Dose Strength ^{a}: | 20 mg | 0 mg |
| Doses tested: | 20 mg, 40 mg, 60 mg | 0 mg |
| Route of Administration: | Oral | Oral |
| Packaging and Labeling: | Upon each visit, the participant will | Upon each visit, the participant will |
| | receive a wallet made of 16 × 3 blister | receive a wallet made of 16 × 3 blister |
| | pockets. Each pocket is filled with a | pockets. Each pocket is filled with a |
| | tablet. The wallet is designed to cover | tablet. The wallet is designed to cover |
| | 16 days of dosing where each line | 16 days of dosing where each line |
| | represents 1 day of administration. | represents 1 day of administration. |
| Dosing Instructions: | The participant is to take 3 tablets a day. | The participant is to take 3 tablets a day. |
| Storage and Handling: | The study interventions can be stored at | The study interventions can be stored at |
| | room temperature. | room temperature. |

^{a} Dose strength refers to CRB-913 as the free drug equivalent.

### Efficacy Assessments

For Part 2, participants should void their bladder before weight measurements are taken. Weight (pounds or kilogram, as applicable) and waist circumference (inches or centimeters, as applicable) will be measured in triplicate using a standard measuring method. A scale calibrated according to the manufacturer's recommendation should be used at all visits for a given participant. A medical-grade scale for weight should be used.

Waist circumference should be measured at the level of the umbilicus and around the top of the iliac crest. The same measuring device should be used at all visits for a given participant. The tape should go around the participant and should be parallel to the floor in the plane that includes the umbilicus and the top of the iliac crest.

### Safety Assessments

### a) Physical Examinations

For Part 1, a full physical exam should be performed at screening and Day 3. A brief physical exam should be performed at admission.

For Part 2, a full physical examination should be performed at screening and EOT and a symptom-directed physical exam at all other visits.

A complete physical examination will include, at a minimum, assessments of the cardiovascular, respiratory, gastrointestinal, and neurological systems. Height and weight will also be measured and recorded.

A brief physical examination will include, at a minimum, assessments of the skin, lungs, cardiovascular system, and abdomen (liver and spleen).

### b) Vital Signs

Blood pressure and pulse rate will be measured in a supine position after resting for 5 minutes.

### c) Electrocardiograms (ECGs)

In Part 1 and Part 2, triplicate 12-lead ECGs will be obtained using an ECG machine that automatically calculates the heart rate and measures PR, QRS, QT, and QTc intervals. ECGs should be performed after the participant has been resting in a supine position for at least 5 minutes, as feasible, and prior to other procedures scheduled at that same time (e.g., blood draws). The evaluation of ECGs will include assessment of changes in the following ECG parameters: heart rate, PR interval, QRS, QT, and QTcF intervals. If ECG results are abnormal, and considered clinically significant by the investigator, additional ECGs should be done at the investigator's discretion. QTc prolongations reported by the site will be assessed according to National Cancer Institute Common Terminology Criteria for Adverse Events Version 5.0.

### Suicidal Ideation and Behavior (SIB) Risk Monitoring

CRB-913 is related to products with an increased risk of suicidal ideation or behavior. Participants being treated with CRB-913 should be monitored appropriately and observed closely for SIB or any other unusual changes in behavior, especially at the beginning and end of the course of intervention, or at the time of dose changes, either increases or decreases. Participants who experience signs of SIB should undergo a risk assessment. All factors contributing to SIB should be evaluated and consideration should be given to discontinuation of the study intervention.

Baseline assessment of suicidal ideation and behavior/intervention-emergent suicidal ideation and behavior will be monitored during CRB-913-02 using C-SSRS assessment.

### Columbia-Suicide Severity Rating Scale (C-SSRS)

The “Baseline/Screening” C-SSRS questionnaire template will be used at the screening visit, and the “Since Last Visit” C-SSRS questionnaire template will be used for other timepoints. Any clinically significant abnormality will be reported as an AE. The results of the C-SSRS assessment from screening will be shared with a qualified mental health professional (e.g., a licensed psychiatrist or clinical psychologist) for confirmation that each participant is eligible for the study. Any participant who develops a new-onset case of SIB during the study will be referred to an appropriate healthcare professional.

### Patient Health Questionnaire-9 (PHQ-9)

Any clinically significant abnormality will be reported as an AE. The results of the PHQ-9 questionnaire (see: Kroenke K, Spitzer R L, Williams J B. The PHQ-9: validity of a brief depression severity measure. J Gen Intern Med. 2001; 16 (9): 606-613) from screening will be shared with a qualified mental health professional (e.g., a licensed psychiatrist or clinical psychologist) for confirmation that each participant is eligible for the study. Any participant who develops a new-onset case of depression during the study will be referred to an appropriate healthcare professional.

### General Anxiety Disorder-7 Questionnaire (GAD-7)

Any clinically significant abnormality will be reported as an AE. The results of the GAD-7 questionnaire (see: Lowe B, Decker O, Müller S, et al. Validation and standardization of the Generalized Anxiety Disorder Screener (GAD-7) in the general population. Med Care. 2008; 46(3):266-274) from screening will be shared with a qualified mental health professional (e.g., a licensed psychiatrist or clinical psychologist) for confirmation that each participant is eligible for the study. Any participant who develops a new-onset case of anxiety during the study will be referred to an appropriate healthcare professional.

### Neurological Examination

Participants in Part 1 and Part 2 will undergo neurological examinations. A full neurological examination will be carried out according to a pre-determined script and will cover central and peripheral neurological disorders by assessing mental status, cranial nerves, motor function, coordination, reflexes, and sensory functions. In addition, a Romberg test will be performed to assess for ataxia. A participant may be referred to an appropriate healthcare professional based on the results of their full neurological examination and the judgement of the investigator.

### Food Noise Questionnaire (FNQ)

The FNQ is a patient-reported outcome instrument developed to assess the intensity and frequency of intrusive or persistent thoughts about food, eating, and appetite. In this study, the FNQ will be used to evaluate changes in “food noise” associated with study intervention in adults with obesity. The FNQ will be completed by participants using a validated eCOA system provided at site. Participants will complete the questionnaire on a site-provided device (e.g., tablet or handheld unit) under supervision of study staff, in a private setting to ensure confidentiality. Participants should complete the questionnaire prior to any study procedures that might bias responses (e.g., before weight measurement, waist measurement, or dosing).

### Dual Energy X-ray Absorptiometry (DXA)

A subset of participants in Part 2 will undergo DXA assessments to evaluate changes in bone mineral density and body composition associated with exposure to study intervention. Scans will include whole-body composition (total and regional lean and fat mass). All scans will be performed using standardized positioning and acquisition protocols on appropriately maintained and calibrated DXA equipment. The same device and software version will be used for each participant whenever feasible. DXA involves minimal radiation exposure. Participants with contraindications to DXA (e.g., pregnancy, inability to lie flat, or body habitus exceeding scanner limits) will be excluded from this sub-study.

### Pharmacokinetics

PK samples will be collected in Part 1 and Part 2. Blood samples will be processed to plasma, divided into 2 approximately equal aliquots, and frozen for future analysis of CRB-913 concentrations. Detailed instructions regarding collection, processing, and storage of samples will be provided to the sites in the form of a laboratory manual. Samples will be shipped to central laboratory for analysis of CRB-913 by GLP-validated liquid chromatography-tandem mass spectrometry methods.

Part 1: To characterize the PK and estimate relative bioavailability of the 80 mg tablet dose, PK samples may be collected at various timepoints from zero to 72 hours post dose.

Part 2: PK samples will be collected at various timepoints, e.g., Part 2 day 1, day 15, day 29, day 43, day 57, day 71, and/or day 85.

### Pharmacodynamics

For Part 2:
Glucose metabolism: non-fasting glucose, insulin, HbA1c
Lipid profile: triglycerides, high-density lipoprotein, low-density lipoprotein, total cholesterol
Adipokines and hormones: leptin, adiponectin, ghrelin, GLP-1, peptide YY, cortisol

Weight will also be assessed at every clinic visit. DXA scan measurement will be performed to assess the degree of preservation of muscle mass versus fat mass.

### Biomarkers

For Part 2:
High-sensitivity C-reactive protein
N-terminal pro-B-type natriuretic peptide

### Multiplicity Adjustment

No multiplicity adjustment will be made for Part 1.

No inferential analyses will be performed for safety assessments.

A hierarchical testing method will be employed to control the overall type 1 error rate for the comparison of each active CRB-913 dose to placebo for the secondary endpoint of change from baseline to Week 12 in absolute weight. Statistical testing will be performed at the 2-sided 0.05 level in the prespecified below order, with formal testing not continuing once non-significance is observed. The prespecified order is:
1. Change in absolute body weight from baseline to Week 12 for CRB-913 60 mg daily versus placebo
2. Change in absolute body weight from baseline to Week 12 for CRB-913 40 mg daily versus placebo
3. Change in absolute body weight from baseline to Week 12 for CRB-913 20 mg daily versus placebo

All other secondary and exploratory endpoints, and supportive analyses, will not be formally tested and no procedures will be implemented to account for multiple comparisons.

### Analysis of Primary Endpoints-Part 1

The primary endpoint for Part 1 is to evaluate the PK parameters of CRB-913.

The PK concentration for CRB-913 versus time will be summarized using nominal timepoints using appropriate statistical descriptors. Individual and mean PK concentrations versus time will be presented graphically. Individual PK concentration for CRB-913 will be listed with nominal and actual times.

The following PK parameters for CRB-913 will be derived using non-compartmental analysis methods:
Maximum plasma concentration (Cₘₐₓ);
Time to Cₘₐₓ (Tₘₐₓ);
Area under the plasma concentration-time curve from time 0 to 24 hours (AUC₀₋₂₄ₕ);
AUC from time 0 to 48 hours (AUC₀₋₄₈h);
AUC from time 0 to the time of the last measurable (positive) concentration (AUCₗₐₛₜ);
AUC from time 0 to infinity (AUC_{0-inf});
Terminal elimination half-life (t_{1/2});
Apparent total clearance (CL/F).

Additional PK parameter will be derived as data permits.

### Analysis of Primary Endpoints—Part 2

The primary endpoint for Part 2 is to evaluate the safety CRB-913. Safety data will be analyzed using the Safety Set and summarized by visit with descriptive summaries based on the treatment actually received.

Safety endpoints will include TEAEs, SAEs, AESIs, clinical laboratory assessments, vital signs, and 12-lead ECGs. Descriptive summaries will be presented, and no inferential statistical analyses are planned for the safety endpoints.

AEs will be coded using the Medical Dictionary for Regulatory Activities (latest version), and data will be summarized by SOC and preferred term. The number and percentage of participants reporting each AE will be summarized by treatment group, as appropriate.

TEAEs are defined as AEs that occurred or worsened following the first administration of study intervention. TEAEs by severity status, relationship to study intervention, SAEs, and AESIs will be summarized. All AEs regardless of treatment emergence will be listed.

Absolute laboratory values, changes from baseline in laboratory values, and laboratory abnormalities will be displayed for each participant and treatment group and summarized over time.

Absolute vital sign values and changes from baseline in vital sign values will be displayed for each participant and treatment group and summarized over time.

Absolute ECG values and changes from baseline in ECG values will be displayed for each participant and treatment group and summarized over time.

### Analyses Supporting Secondary Objectives

The secondary endpoint for Part 1 is to evaluate the safety of a single dose of CRB-913.

For part 2, the key secondary endpoint is the change from baseline in absolute body weight at Week 12. Weight will be summarized using descriptive statistics and graphs (mean±SD) for each treatment group at each timepoint.

### Estimands

The estimand for the Part 2 key secondary efficacy analysis is a hypothetical strategy estimand and is described below:
The population for the study is defined through appropriate inclusion/exclusion criteria to reflect the targeted participant population. The analysis population is based on the FAS and includes all randomized participants who received at least 1 dose of study intervention.
The variable of interest is the change from baseline in body weight at Week 12.
The ability to evaluate treatment effect using the variable of interest may be affected by intercurrent events. Receipt of prohibited medications for the treatment of weight during the treatment period is an intercurrent event of interest because participants' use of such medications may make the treatment effect difficult to interpret. Additionally, permanent treatment discontinuation would also affect the interpretation of the treatment effect. As the evaluation of the key secondary endpoint may be affected by these intercurrent events, data obtained from a participant following the initiation of a prohibited medication for the treatment of weight or after permanent treatment discontinuation will be excluded from the primary analysis.
The population level summary measure is the difference in least squares (LS) means for the change in weight from baseline to Week 12 estimated from a Mixed Model for Repeated Measures (MMRM) model. The MMRM model will include terms for treatment, visit, sex, treatment by visit interaction, participant as a random effect, and baseline weight. Results will be expressed as the difference between each CRB-913 dose and placebo, along with 95% confidence intervals (Cls) and p-values.

### Supplementary Analyses

As a supplemental analysis to the key secondary efficacy analysis, the change from baseline to Week 12 in absolute body weight will be evaluated using an analysis of covariance (ANCOVA) model with a fixed effect for the treatment group and covariates of baseline body weight and sex. Analyses will be performed using the observed data with no imputation for missing values.

### Other Efficacy Analyses

Part 2 continuous efficacy endpoints will be analyzed using similar methods as in the key secondary efficacy analysis. The 2-sided 95% CI for LS means will be provided. Additional sensitivity and supplemental analyses may be carried out under secondary estimands and/or various assumptions for missing data.

### Analyses Supporting Pharmacokinetic Objective for Part 2

The secondary PK endpoint for Part 2 is to evaluate the PK concentration of CRB-913 overtime and PK parameters.

The PK concentration for CRB-913 versus time will be summarized using nominal timepoints using appropriate statistical descriptors. Individual and mean PK concentrations versus time will be presented graphically. Individual PK concentration for CRB-913 will be listed with nominal and actual times.

### Analyses Supporting Exploratory Objectives

The exploratory endpoints will be analyzed similarly to the primary and secondary endpoints.

### DXA Sub-study Analyses

The DXA analysis population will include all participants enrolled in the DXA sub-study who have a valid baseline DXA measurement and at least 1 postbaseline DXA assessment. The exploratory endpoint will be whole-body composition parameters (e.g., total lean mass, fat mass, appendicular lean mass, percent body fat).

DXA endpoints will be summarized descriptively by treatment group and visit. Summary statistics will include n, mean, SD, median, minimum, and maximum, as well as change from baseline with 95% Cls.

Exploratory between-group comparisons may be conducted using an ANCOVA model, with treatment group as a fixed effect and baseline value as a covariate. Repeated-measures models may also be employed to evaluate longitudinal patterns over time, assuming missing-at-random data. No formal multiplicity adjustment is planned for exploratory DXA endpoints.

Artifacts and vertebra exclusions will follow prespecified central reading criteria. All DXA data will be reviewed by a blinded central reader to ensure standardization across sites.

### Other Safety Analyses

The number and percentage of participants experiencing treatment-emergent suicidal behavior or ideation, anxiety, and depression as determined from the C-SSRS, PHQ-9, and GAD-7 will be summarized. The results of the participants' neurological examinations over time will be listed. The FNQ outcomes will be summarized.

### Example 9: Additional Dose Screening

### Elevated Dose Screening

CRB-913 in a total daily from 80 mg to 300 mg as single or multiple doses are evaluated. Administration can include:
80 mg dose administered as a single daily dose
80 mg dose administered as two daily doses
80 mg dose administered once a week
80 mg dose administered Q2D
80 mg dose administered Q3D
100 mg dose administered as a single daily dose
100 mg dose administered as two daily doses
100 mg dose administered once a week
100 mg dose administered Q2D
100 mg dose administered Q3D
125 mg dose administered as a single daily dose
125 mg dose administered as two daily doses
125 mg dose administered once a week
125 mg dose administered Q2D
125 mg dose administered Q3D
150 mg dose administered as a single daily dose
150 mg dose administered as two daily doses
150 mg dose administered once a week
150 mg dose administered Q2D
150 mg dose administered Q3D
175 mg dose administered as a single daily dose
175 mg dose administered as two daily doses
175 mg dose administered once a week
175 mg dose administered Q2D
175 mg dose administered Q3D
200 mg dose administered as a single daily dose
200 mg dose administered as two daily doses
200 mg dose administered once a week
200 mg dose administered Q2D
200 mg dose administered Q3D
225 mg dose administered as a single daily dose
225 mg dose administered as two daily doses
225 mg dose administered once a week
225 mg dose administered Q2D
125 mg dose administered Q3D
250 mg dose administered as a single daily dose
250 mg dose administered as two daily doses
250 mg dose administered once a week
250 mg dose administered Q2D
250 mg dose administered Q3D
275 mg dose administered as a single daily dose
275 mg dose administered as two daily doses
275 mg dose administered once a week
275 mg dose administered Q2D
275 mg dose administered Q3D

An ascending dose regimen, e.g., as described herein, may be utilized until the target total daily dose is reached. Each dose will be continually administered for a period of 7 to 28 days, 1 to 12 weeks, or 1 to 12 months. Study parameters as outlined in Example 7 can be used to assess the elevated doses and dosing regimens.

CRB-913 in a total daily dose of 300 mg, 400 mg, and 600 mg as single or multiple doses are evaluated. Administration can include:
300 mg dose administered as a single daily dose
300 mg dose administered as two daily doses
300 mg dose administered once a week
300 mg dose administered Q2D
300 mg dose administered Q3D
400 mg dose administered as a single daily dose
400 mg dose administered once a week
400 mg dose administered Q2D
400 mg dose administered Q3D
600 mg dose administered as a single daily dose
600 mg dose administered once a week
600 mg dose administered Q2D

600 mg dose administered Q3DAn ascending dose regimen, e.g., as described herein, may be utilized until the 300 mg, 400 mg, or 600 mg dose is reached. The 300 mg, 400 mg, or 600 mg dose will be continually administered for a period of 7 to 28 days, 1 to 12 weeks, or 1 to 12 months. Study parameters as outlined in Example 7 can be used to assess the elevated doses and dosing regimens.

### Low Dose Screening

CRB-913 in a total daily dose of 5 mg, 8 mg, 10 mg, 12 mg, 15 mg, and 18 mg, as single or multiple doses, will also be evaluated. Administration will include daily administration: once a day, twice a day, or three times a day, and weekly administration: once a week, Q2D, or Q3D. Each elected dose will be continually administered for a period of 7 to 28 days, 1 to 12 weeks, or 1 to 12 months. Study parameters as outlined in Examples 7 and 8 can be used to assess the elevated doses and dosing regimens.

### Example 10: CRB-913 Tablet Formulations

### Formulation Development Studies

The tablet formulation focused on developing a compacted tablet drug product using the lead 50/50 CRB-913/HPMCAS-M SDD formulation. This was accomplished by utilizing two relevant tablet excipient blends, as described by Table 11, compressed into 100 mg compacts which were evaluated for manufacturability, chemical compatibility, and disintegration of tablets.

TABLE 11
Formulation Screening Blends

| | | Excipient | Excipient |
|---|---|---|---|
| | | Blend 1 | Blend 2 |
| Function | Ingredient | Composition (%) | |
| Intermediate | 50/50 CRB-913/HPMCAS-M SDD | 50.0 | 50.0 |
| Ductile Filler | Microcrystalline Cellulose | 21.0 | 20.5 |
| Brittle Filler | Lactose Monohydrate | 21.0 | NA |
| Brittle Filler | Mannitol | NA | 20.5 |
| Disintegrant | Croscarmellose Sodium | 6.0 | NA¹ |
| Disintegrant | Crospovidone | NA | 6.0 |
| Glidant | Silicon Dioxide | 1.0 | 1.0 |
| Lubricant | Magnesium Stearate | 1.0 | NA |
| Lubricant | Sodium Stearyl Fumarate | NA | 2.0 |

The compaction simulator studies were performed to assess manufacturability of the SDD blends. Blends were manufactured by mixing for 1 minute in a Resodyn LabRAM II at 40 G. The Styl'One Evo Compaction Simulator was used to compress each blend to a target weight of 100 mg and a tablet tensile strength of 2.5 MPa. Ejection forces were within the expected range for this tablet size. No punch sticking was observed during or after the process. Die filling exhibited minimal variation, indicating consistent performance.

Disintegration testing was performed for each compact blend using a disintegration apparatus and 37 °C, 0.01N HCl, pH 2.0 media. Both compact blends disintegrated quickly.

### Chemical Stability of the Two Compacts.

The compact blends were placed in stability chambers and tested for chiral purity as well as assay and related substances. The chiral purity of compact blend samples was unchanged after 4 weeks relative to initial testing, showing no significant change in enantiomer content for all conditions. The excipient blend 1 had no detectable impurities after 4 weeks of storage. Excipient blend 2 had some levels of impurities detected after 4 weeks at all conditions.

Overall, excipient blend (1 and 2) showed acceptable compaction and fast disintegration. The Excipient blend 2 showed impurity growth at 4 weeks compared to no impurity growth for the excipient blend 1. Notably, excipient blend 1 was the identified as optimal for progression.

### Dosage Form

CRB-913 tablets were formulated as immediate release tablets in 20 mg and 50 mg strengths. The CRB-913 tablet is composed of a spray dried dispersion (SDD) containing 50% w/w CRB-913 drug substance and hypromellose acetate succinate. The quality reference and function of each component is presented in the Table 12. The polymer hypromellose acetate succinate (inactive ingredient) used for CRB-913 SDD was within the limits for oral route of administration, as listed in the FDA inactive ingredient database. The two solvents, methanol and dichloromethane, are removed from the drug product during the spray drying process and controlled within the ICH Q3C recommended limits. All the other excipients used in CRB-913 tablet are compendial excipients which comply with the requirement of USP/NF, Ph. Eur. OPADRY® II Brown was used as a film coating system for CRB-913 tablet drug product (Table 13).

TABLE 12
Tablet Composition

| | | | |
|---|---|---|---|
| | Quality | | Composition |
| Component | Standard | Function | (w/w %) |

CRB-913 SDD

| | | | |
|---|---|---|---|
| CRB-913 | In-house | Active | 50 |
| Hypromellose acetate | NF, Ph. Eur. | Dispersion | 50 |
| Succinate (HPMCAS-M) | | polymer | |
| Dichloromethane (DCM) | NF, Ph. Eur. | Spray drying | —3 |
| | | solvent | |
| Methanol (MeOH) | USP/NF, Ph. Eur. | Spray drying | —3 |
| | | solvent | |

| | Quality | | Composition | | | |
|---|---|---|---|---|---|---|
| Component | Standard | Function | % w/w | mg/tab | % w/w | mg/tab |

CRB 913 Granulation and Compression

| | | | | | | |
|---|---|---|---|---|---|---|
| CRB 913 SDD (50% w/w) | In-house | Intermediate | 20.00 | 40.0 | 50.00 | 100.00 |
| Microcrystalline Cellulose | NF, Ph. Eur. | Filler | 36.25 | 72.5 | 21.25 | 42.5 |
| Lactose Monohydrate | USP/NF, Ph. Eur. | Filler | 36.25 | 72.5 | 21.25 | 42.5 |
| Croscarmellose Sodium | NF, Ph. Eur. | Disintegrant | 6.00 | 12.0 | 6.00 | 12.0 |
| Silicon Dioxide | USP/NF, Ph. Eur. | Glidant | 1.00 | 2.0 | 1.00 | 2.0 |
| Magnesium Stearate | NF, Ph. Eur. | Lubricant | 0.50 | 1.00 | 0.50 | 1.00 |

| | | | | |
|---|---|---|---|---|
| Tablet Core | 100.00 | 200.00 | 100.00 | 200.00 |

CRB 913 Coating System

| | | | | | | |
|---|---|---|---|---|---|---|
| Opadry II Brown | Manufacturer | Film coating | 15¹ | 6² | 15¹ | 6² |
| | standard | system | | | | |
| Distilled Water | In-house | Solvent | 85¹ | —3 | 85¹ | —³ |

| | | | | |
|---|---|---|---|---|
| Final Coated Tablet | 100.00 | 206.0 | 100.00 | 206.0 |

¹Represents composition of the coating suspension, not overall coated tablet;
²Represents 3.0 wt. % coat weight nominate on the tablet core;
³Removed during processing and not part of the finished product.

TABLE 13
Qualitative Composition of OPADRY ® II Brown (85F265096)

| Component | Compendial Reference |
|---|---|
| Polyvinyl Alcohol (PVA) | USP, FCC, Ph. Eur., JPE, ChP, GB |
| Polyethylene Glycol (PEG)/ | USP, FCC, Ph. Eur., JECFA, JP |
| Macrogol | |
| Titanium Dioxide | USP, FCC, Ph. Eur., JP, JSFA, ChP, GB |
| Talc | USP, FCC, Ph. Eur., JP, JECFA, ChP |
| Ferric Oxide Red | NF, JPE, JECFA, ChP |
| Ferric Oxide Yellow | NF, JPE, JECFA, ChP |
| Ferrosoferric Oxide NF/Black | JPE, JECFA, ChP |
| Iron Oxide | |

JP = Japanese Pharmacopoeia,
NF = National Formulary,
Ph. Eur. = European Pharmacopoeia,
USP = United States Pharmacopoeia,
JECFA = Joint Evaluation Committee on Common Additives,
FCC = Food Chemical Index,
ChP = Chinese Pharmacopoeia,
JPE = Japanese Pharmaceutical Excipients,
GB = China National Standard

### Development of Manufacturing Process

Based on the tablet development study, excipient blend 1 containing lactose and microcrystalline cellulose (as the fillers), croscarmellose sodium (as the disintegrant) and magnesium stearate (as the lubricant) was identified. Different ratios were used for the 20 mg and 50 mg tablets. The composition of both active formulations are summarized in Table 12.

The pre-granulation blends true density and flow was characterized using the following techniques: Accupyc, bulk and tapped density, and shear cell. Flow measurements indicate 20 mg tablet blend flows better and is more dense than 50 mg tablet blend. This is expected based on the SDD concentration.

Granulation was performed using a Gerteis Mini-Pactor with an inline mill. A target ribbon tensile strength of 0.6 MPa was selected for both 20 mg and 50 mg tablet blends. The ribbon solid fraction remained consistent throughout the run for both the 20 mg and 50 mg tablet blends.

The extra-granular excipient quantities were adjusted based on the amount of in-going granulation.

The flow of the final blend was characterized via bulk and tapped density, and shear cell. The Final Flow Characteristics (FFC) and Cohesion factors for both the 20 mg and 50 mg tablet formulations had sufficient flow for tableting.

The 20 mg and 50 mg tablets were compressed at the target weight across a range of hardness values to identify a target hardness that would be robust with respect to performance, image matching, and friability. Tablets were manufactured using a Styl'One Evo compaction simulator at and around the proposed target tensile strengths—2.0 MPa for the 20 mg tablet and 3.0 MPa for the 50 mg tablet. These tensile strength targets yielded tablets with similar thicknesses, within ±0.2 mm, to meet visual match criteria. The differing tensile strength requirements reflect the variation in compressibility of the final blends for the 20 mg and 50 mg tablets, due to differences in the concentrations of brittle components and SDD.

Disintegration times were rapid (at ≤5 minute) for all hardnesses tested of both 20 mg and 50 mg tablets. Both tablet dose strengths displayed acceptable friability and remained <0.3% weight loss on 100 drops at all hardnesses tested. Tablet thicknesses were within +/−0.2 mm for all tablet hardness tested of the 20 mg and 50 mg tablets indicating tablets were visually matching. Tablet hardnesses of 9.0 and 14.0 corresponding to tensile strengths of 2.0 and 3.0 MPa were selected for the 20 mg and 50 mg tablets, respectively.

All bulk compressions were performed using a Korsch XL100 tablet press. A hardness bracketing experiment was performed prior to bulk compression to ensure the target tablet tensile strength was robust with respect to disintegration time, friability, and image matching criteria. For the hardness bracketing experiment, 20 mg and 50 mg tablets were compressed at their low, target, and high tensile strengths for approximately 2 minutes and tested for disintegration in 0.01N HCl media and friability. Disintegration, image, and friability results were consistent with those found on the compaction simulator for both tablet dose strengths.

The bulk compression was performed targeting a tablet tensile strength of 3.0 MPa and a tablet weight of 200 mg. In-process testing of the tablet weight, thickness, and hardness was performed every 10 minutes. During compression, three tablet samples were taken during the beginning, middle, and end of the run. These tablets were tested for disintegration time in a 0.01N HCl media and friability. The disintegration times for all dosages were less than 5 minutes. Additionally, one hundred tablets were visually inspected for defects after completing the bulk compression.

The tablets were coated on a Vector LDCS5 pan coater using an OPADRY® II Brown coating system. The coating suspension was prepared using an overhead agitator. The suspension composition is 15% OPADRY® II Brown and 85% water (deionized). A weight gain of 3.0 wt % with an acceptable range of 2.5-4.0 wt % was selected in order to ensure complete coating of the core tablet and also to maintain consistency between the active and placebo tablets. Processing was conducted at a scale representative of the intended clinical manufacturing scale to enable direct transfer of coating conditions.

Coated tablets were tested for disintegration in a 0.01N HCl media per USP <701> at 37 °C±0.5 °C. No differences were observed in disintegration of coated as well as uncoated tablets. Therefore, the coating was deemed successful. Both strengths (20 mg and 50 mg) of CRB-913 tablets were coated with non-functional film coat to achieve identical appearance (e.g., same size, shape, weight, thickness, and color).

Sink dissolution testing was performed on uncoated, 20 and 50 mg CRB-913 tablets as a function of tensile strength. Tablets used for dissolution as a function of tensile strength were made in a compaction simulator during the demonstration run. Results for the 20 mg uncoated tablets showed consistent performance for centerline bracketing tensile strengths, reaching 80% dose within 10 minutes. The 50 mg uncoated tablets reached 80% dose between 25 and 30 minutes and showed a minor impact from tensile strength; with the high (3.5 MPa) tensile strength showing slightly slower dissolution for the first 30 minutes. The 50 mg tablets performance was consistent after 30 minutes.

Content uniformity (CU), assay, and related substances of CRB-913 film coated tablets (20 mg and 50 mg) were evaluated via HPLC. For both 20 mg and 50 mg strengths, assay values were consistent across replicate analysis. No related substances were detected. For both 20 mg and 50 mg strengths, CU acceptance values (AV) were below the acceptance limit of 15 (for N=10).

The chiral purity of CRB-913 film coated tablets (20 mg and 50 mg) was evaluated by HPLC. Comparable purity was observed for the 20 mg and 50 mg CRB-913 tablets to the CRB-913 control.

CRB-913 20 mg and 50 mg tablets were analyzed by PXRD to evaluate CRB-913 crystallization over time. No CRB-913 related crystalline peaks were identified.

### Manufacturing Process

The tablet manufacturing process is outlined in FIG. 15.

### Example 11: CRB-913 for Weight Maintenance

CRB-913 is a peripherally restricted cannabinoid type 1 receptor inverse agonist that has shown preclinical efficacy in mice with diet-induced obesity as a monotherapy and in combination with incretin analogs. CRB-913 was also investigated as a maintenance therapy following semaglutide-induced weight loss.

The introduction of incretin analogs has transformed the treatment of obesity and given rise to a range of different therapeutic options capable of inducing significant weight loss within several months (Chakhtoura M, Haber R, Ghezzawi M, Rhayem C, Tcheroyan R, Mantzoros C S. Pharmacotherapy of obesity: an update on the available medications and drugs under investigation. EClinicalMedicine. 2023; 58:101882). However, as with earlier generations of weight loss medications, discontinuation of incretin analogs can result in weight regain; therefore, continued long-term administration is required to sustain treatment benefits (Wu H, Yang W, Guo T, Cai X, Ji L. Trajectory of the body weight after drug discontinuation in the treatment of anti-obesity medications. BMC Medicine. 2025; 23(1):398; Rubino D, Abrahamsson N, Davies M, et al. Effect of continued weekly subcutaneous semaglutide vs placebo on weight loss maintenance in adults with overweight or obesity: the STEP 4 randomized clinical trial. JAMA. 2021; 325(14):1414-1425; Berg S, Stickle H, Rose S J, Nemec E C. Discontinuing glucagon-like peptide-1 receptor agonists and body habitus: a systematic review and meta-analysis. Obes Rev. 2025; 26(8):e13929). This long-term usage may be limited by issues related to tolerability, loss of muscle mass and function, affordability, and supply chain or manufacturing challenges (Mechanick J I, Butsch W S, Christensen S M, et al. Strategies for minimizing muscle loss during use of incretin-mimetic drugs for treatment of obesity. Obes Rev. 2025; 26(1):e13841; Karagiannis T, Bekiari E, Tsapas A. Socioeconomic aspects of incretin-based therapy. Diabetologia. 2023; 66(10):1859-1868; Whitley H P, Trujillo J M, Neumiller J J. Special report: potential strategies for addressing GLP-1 and dual GLP-1/GIP receptor agonist shortages. Clin Diabetes. 2023; 41(3):467-473). As a result, real-world treatment adherence is suboptimal, with approximately one-third of patients persisting with prescribed regimens after 1 year of treatment (Gleason P P, Urick B Y, Marshall L Z, Friedlander N, Qiu Y, Leslie R S. Real-world persistence and adherence to glucagon-like peptide-1 receptor agonists among obese commercially insured adults without diabetes. J Manag Care Spec Pharm. 2024; 30(8):860-867).

The implementation of an induction-maintenance treatment strategy may offer a potential solution to these challenges. Weight loss would be induced with a time-limited course of incretin analog therapy, followed by transition to a different class of medication for chronic weight loss maintenance. The success of this approach is dependent on identifying a maintenance therapy that is safe and well tolerated, easy to administer, widely accessible, and of sufficient efficacy.

Targeting the endocannabinoid system via cannabinoid type 1 receptor (CB1) inverse agonism is a clinically validated approach that may offer an alternative route to weight loss (O'Sullivan S E, Yates A S, Porter R K. The peripheral cannabinoid receptor type 1 (CB1) as a molecular target for modulating body weight in man. Molecules. 2021; 26 (20): 6178). However, previous clinical development efforts for this class of drugs were halted owing to the risk of neuropsychiatric events, which led to the market withdrawal of the first-generation CB1 inverse agonist, rimonabant (Cohen Y, Kolodziej A, Morningstar M. Seventeen years since rimonabant's downfall: reassessing its suicidality risk profile. Obesity. 2024; 32(7):1235-1244).

With the objective of mitigating this risk, a new generation of CB1 inverse agonists has been developed to minimize central nervous system penetration (Cinar R, lyer M R, Kunos G. The therapeutic potential of second and third generation CB1R antagonists. Pharmacol Ther. 2020; 208:107477; Knop F K, Kunos G, Dicker D, et al. Efficacy and safety of monlunabant in adults with obesity and metabolic syndrome: a double-blind, randomized, placebo-controlled, phase 2a trial. Lancet Diabetes Endocrinol. 2025; 13(11):911-923). CRB-913 is an orally administered, peripherally restricted CB1 inverse agonist with markedly lower brain penetration than rimonabant. In a mouse model of diet-induced obesity (DIO), CRB-913 monotherapy induced weight loss and improved metabolic outcomes with minimal effects on muscle mass (Morningstar M, Kolodziej A, Ferreira S, Blumen T, Brake R, Cohen Y. Novel cannabinoid receptor 1 inverse agonist CRB-913 enhances efficacy of tirzepatide, semaglutide, and liraglutide in the diet-induced obesity mouse model. Obesity. 2023; 31(11):2676-2688). Furthermore, CRB-913 exerted an additive effect on weight loss when co-administered with the incretin analogs liraglutide, semaglutide, or tirzepatide, which suggests potential utility as a combination therapy (FIG. 17).

An induction-maintenance protocol using the DIO mouse model was devised to evaluate the potential of CRB-913 in a third preclinical setting as a maintenance therapy following weight loss induction with incretin analogs. This sequential approach to treatment aims to address the concerns associated with long-term incretin analog use by transitioning to a mechanistically distinct therapy that maintains long-term weight loss benefits.

### Methods

A diet-induced obesity mouse protocol was used to evaluate an induction-maintenance regimen. Mice were randomized to receive: (1) vehicle for 42 days; (2) CRB-913 for 42 days; (3) semaglutide for 42 days; (4) semaglutide for 21 days then CRB-913 for 21 days; or (5) semaglutide for 21 days then vehicle for 21 days. Body weight and composition, fat deposition, and liver histology were assessed.

### DIO Mouse Generation

A high-fat diet was used to induce obesity in mice as described previously (Morningstar M, Kolodziej A, Ferreira S, Blumen T, Brake R, Cohen Y. Novel cannabinoid receptor 1 inverse agonist CRB-913 enhances efficacy of tirzepatide, semaglutide, and liraglutide in the diet-induced obesity mouse model. Obesity. 2023; 31(11):2676-2688). Briefly, 5-week-old male C57BL/6J mice were housed (5 mice/cage) in a controlled environment (20-24 °C with 30-70% relative humidity) with a 12-hour dark/light cycle. Mice were fed a 60% fat diet for at least 15 weeks, had obesity (mean [range] weight ~46 g [44.8-48.8]), demonstrated mild to moderate hyperglycemia (fasting blood glucose ≥120 mg/dL), and showed impaired glucose tolerance. Two weeks before studying drug treatment initiation, mice received placebo once daily (QD) orally (PO) alongside the high-fat diet to habituate them to the dosing procedure and minimize stress-related weight fluctuations. Mice with unstable body weight following habituation were excluded.

### Treatment Formulation

CRB-913 was produced by Corbus Pharmaceuticals Inc., and semaglutide was sourced from Aladdin Scientific. CRB-913 formulations were freshly prepared every 3 days as a 1.0 mg/mL CRB-913 suspension in 0.5% hydroxypropyl methylcellulose acetate succinate (HPMC-AS). Phosphate-buffered saline (pH 7.4) containing 1.0 mg/mL HMPC-AS (dosed at 10 mg/kg) was used as vehicle. Semaglutide formulations were diluted in saline from frozen concentrate (1.00 mg/mL, stored at −80 °C) to 1.0 nmol/mL the day before dosing. All formulations were stored at 2-8 °C.

### Study Design

Mice (n=10 per group) were randomized 1:1:1:1:1 to one of five treatment groups. The VEH/VEH group was administered vehicle QD PO for 42 days. The CRB-913/CRB-913 group received CRB-913 10 mg/kg QD PO for 42 days. The SEMA/SEMA group received 10 nmol/kg semaglutide once every 3 days (Q3D) subcutaneously (SC) for 42 days. Two sequential treatment groups were included: the SEMA/CRB-913 group received semaglutide 10 nmol/kg Q3D SC for 21 days followed by CRB-913 10 mg/kg QD PO for 21 days; the SEMA/VEH group received semaglutide 10 nmol/kg Q3D SC for 21 days followed by vehicle QD PO for 21 days (FIG. 9). Groups were balanced based on body weight and serum levels of alanine aminotransferase (ALT), low-density lipoprotein cholesterol (LDL-C), and glucose. The study director supervised the allocation of animals in accordance with the protocol. Blinding of study personnel to treatment groups was not implemented, as it was considered unnecessary for ensuring accurate measurement and reporting of study endpoints.

### Food Intake and Body Weight Measurements

Food pellets were provided ad libitum, and food intake was measured daily at the start of each dark cycle. Food consumption was recorded at the cage level and average food consumption per mouse was calculated (total food consumption divided by the number of animals per cage). Individual body weights were measured each day after food intake measurements but before treatment administration. Food consumption is reported up to day 40, and body weight measurements are reported up to day 41 before overnight fasting.

### Dual-Energy X-Ray Absorptiometry

Baseline dual-energy X-ray absorptiometry (DEXA) body composition measurements from 10 randomly selected animals were obtained 8 days before treatment initiation using an InAlyzer densitometer (MEDIKORS). Body composition was then assessed for all animals in each arm on day 41. The following data were collected: weight of lean tissue (lean mass, g), weight of fat tissue (fat mass, g), bone mineral density (g/cm²), and bone mineral content (g). Mice were anesthetized by isoflurane inhalation during testing.

### Necropsy, Blood, and Tissue Collection Procedure

On day 42, following an overnight fast, animals were euthanized 2 hours post dose using CO₂ inhalation. Immediately after euthanasia, animals were perfused intracardially with ice-cold saline. Whole blood samples were collected for serum analysis of ALT, aspartate aminotransferase (AST), and LDL-C. Liver and body weights were recorded. The left lateral lobe of the liver was fixed and processed for histological evaluation using hematoxylin and eosin (H&E) staining. Adipose tissues, including brown adipose tissue (scapular) and white adipose depots (perirenal, epididymal, and inguinal) were also collected and weighed.

### Plasma and Tissue Analysis

Serum ALT, AST, and LDL-C concentrations were analyzed using a 7180 Clinical Analyzer (Hitachi). Blood glucose levels were measured using the ACCU-CHEK Active blood glucose meter (Roche Diagnostics Ltd). Hepatic tissue analysis included histological evaluation of ballooning degeneration, steatosis, and inflammation, which were scored according to the validated Pathology Committee of the Nonalcoholic Steatohepatitis Clinical Research Network scoring system (Kleiner D E, Brunt E M, Van Natta M, et al. Design and validation of a histological scoring system for nonalcoholic fatty liver disease. Hepatology. 2005; 41(6):1313-1321; Hjelkrem M, Stauch C, Shaw J, Harrison S A. Validation of the non-alcoholic fatty liver disease activity score. Aliment Pharmacol Ther. 2011; 34(2):214-218). The nonalcoholic fatty liver disease activity score (NAS) represents the sum of the scores for steatosis (0-3 points), lobular inflammation (0-3 points), and hepatocyte ballooning (0-2 points). A NAS of 5 or above in humans is strongly associated with a diagnosis of metabolic dysfunction-associated steatohepatitis, whereas a score of 2 or below is not.

### Statistical Analysis

Statistical analyses were performed using GraphPad Prism version 10.3.1 (GraphPad Software). Data are presented as mean±standard error of the mean (SEM). Group comparisons were conducted using analysis of variance (ANOVA). When ANOVA indicated a significant effect (p<0.05), Dunnett's post hoc test was used to compare treatment groups with the control.

### Results

Switching from semaglutide induction (10 nmol/kg/day) to CRB-913 maintenance (10 mg/kg/day) resulted in an additional 3.5% absolute weight loss versus continuous semaglutide at day 41 (p<0.01). In contrast, switching from semaglutide induction to vehicle maintenance resulted in rapid and complete weight regain. Dual-energy X-ray absorptiometry scans revealed that additional weight loss with CRB-913 maintenance therapy was associated with reduced fat levels compared with continuous semaglutide treatment; liver histology improvements followed a similar trend.

### CRB-913 Demonstrates Additional Weight Loss Following Induction by Semaglutide

An induction-maintenance protocol (FIG. 9) was designed to evaluate if CRB-913 could maintain weight loss after an induction period of semaglutide therapy. Doses of CRB-913 (10 mg/kg) and semaglutide (10 nmol/kg Q3D) were selected based on previous findings (Morningstar M, Kolodziej A, Ferreira S, Blumen T, Brake R, Cohen Y. Novel cannabinoid receptor 1 inverse agonist CRB-913 enhances efficacy of tirzepatide, semaglutide, and liraglutide in the diet-induced obesity mouse model. Obesity. 2023; 31(11):2676-2688), which indicated that both doses confer similar levels of weight loss. This intermediate degree of weight change was considered optimal for detecting changes in weight-related endpoints following the treatment crossover. By day 22, the CRB-913/CRB-913 group had a mean body weight reduction of 19.2% (SEM, 1.05%; FIG. 10). CRB-913-associated weight loss continued to progress throughout the study, reaching a mean reduction of 22.8% (SEM, 1.02%) by day 41. The SEMA/SEMA group had a mean reduction of 13.7% (SEM, 0.51%) by day 22 (corresponding to day 3 in the Q3D dosing regimen), culminating at 13.6% (SEM, 0.50%) on day 41. The SEMA/CRB-913 group had a mean weight loss of 17.1% (SEM, 1.03%) by day 41, representing a 3.5% greater absolute reduction versus the SEMA/SEMA group (p<0.01). In contrast, the SEMA/VEH group had a complete and rapid regain of body weight from day 22, after discontinuation of semaglutide and its replacement with vehicle.

Changes in body weight were accompanied by changes in food intake (FIG. 11). Compared with the VEH/VEH group, all treatment regimens resulted in an initial marked reduction in food intake during the first 2 weeks. After this point, intake stabilized for the CRB-913/CRB-913 and SEMA/CRB-913 groups to a level that remained significantly below the VEH/VEH group until day 40. In the SEMA/SEMA group, oscillations in food intake continued throughout the induction and maintenance periods, reflecting the Q3D dosing schedule. In the SEMA/VEH group, intake increased as soon as semaglutide treatment was withdrawn, with no significant differences observed between the VEH/VEH and SEMA/VEH group during the maintenance phase.

### CRB-913 Induces Further Fat Reduction Following Semaglutide

DEXA scan measurements at the end of study revealed that mice in the CRB-913/CRB-913, SEMA/SEMA, and SEMA/CRB-913 treatment groups had significantly lower levels of fat mass than the VEH/VEH group (p<0.001 for all; FIG. 12A). The largest difference was observed in the CRB-913/CRB-913 group, which had a mean fat mass that was 10.2 g lower than the VEH/VEH group (FIG. 12B). Similarly, mean fat mass was 9.6 g lower in the SEMA/CRB-913 group than in the VEH/VEH group. In the SEMA/SEMA and SEMA/VEH groups, the differences versus vehicle were less substantial (−6.0 g and −2.0 g, respectively). Small differences in lean mass were observed between the VEH/VEH group and all treatment groups (FIG. 12A and FIG. 12B); mean lean mass was 2.3 g and 1.9 g lower in the CRB-913/CRB-913 and SEMA/SEMA groups, respectively (p<0.05) (FIG. 12C and FIG. 12D).

### Liver Biomarkers Mirror Weight Loss Effects

We next compared the effects of treatment on liver pathology steatosis markers. Mean liver organ weight at the end of study was significantly lower in all active treatment groups (FIG. 13A; p<0.001 for all groups vs. VEH/VEH), with the trends across groups mirroring the changes in body weight. Mice in the VEH/VEH group showed clear signs of severe steatohepatitis, including steatosis (mean score, 1.9), ballooning (mean score, 2.0), and lobular inflammation (mean score, 1.6), resulting in an overall NAS of 5.5 (SEM, 0.17; FIG. 13B). In contrast, mice in the CRB-913/CRB-913 and SEMA/SEMA groups had significantly lower NAS (means of 3.2 [p<0.01] and 2.7 [p<0.001], respectively) owing to significantly lower levels of steatosis, ballooning degeneration, and inflammation (FIGS. 13C-13E). Notably, the SEMA/CRB-913 group had the lowest mean NAS of 1.6, and markedly low levels of inflammation (mean inflammation score, 0.1), indicating that liver pathology was below the threshold typically associated with liver disease (Kleiner D E, Brunt E M, Van Natta M, et al. Design and validation of a histological scoring system for nonalcoholic fatty liver disease. Hepatology. 2005; 41 (6): 1313-1321). Mice in the SEMA/VEH group exhibited substantial liver pathology, with a NAS that was not significantly different to the VEH/VEH group (mean NAS, 4.2 vs. 5.5).

The improvements in liver pathology corresponded with reductions in AST and ALT, biomarkers of liver inflammation and/or damage. AST and ALT concentrations were significantly lower in the CRB-913/CRB-913 and SEMA/SEMA groups than the VEH/VEH group (p<0.05 for all). Although AST concentrations in the SEMA/CRB-913 group did not differ significantly from those in the VEH/VEH group, ALT levels were significantly reduced to a level similar to those in the CRB-913/CRB-913 and SEMA/SEMA groups (p<0.05). AST and ALT concentrations in the SEMA/VEH group were similar to those in mice treated with vehicle continuously (VEH/VEH) (FIGS. 13F and 13G).

### CRB-913 Leads to Reduction in Fat Deposits

Fat deposits were weighed on day 42 (perirenal, epididymal, and inguinal white adipose tissue, and brown adipose tissue). Mice in the CRB-913/CRB-913 and SEMA/CRB-913 groups had significantly lower levels of all four adipose tissue types by the end of study compared with the VEH/VEH group (FIG. 14). In contrast, continuous semaglutide treatment (SEMA/SEMA) had negligible impact on epididymal and inguinal white adipose tissue levels and the SEMA/VEH group showed no difference in fat deposits versus the VEH/VEH group.

### Conclusions

In the DIO mouse model, induction treatment with semaglutide resulted in an expected rapid reduction in body weight that plateaued during maintenance period (days 22-42). Discontinuation of semaglutide followed by vehicle resulted in rapid weight regain, with mice returning to baseline weight within 3 weeks, similar to the trends reported in the STEP 1 observational clinical study (Wilding J P H, Batterham R L, Davies M, et al. Weight regain and cardiometabolic effects after withdrawal of semaglutide: the STEP 1 trial extension. Diabetes Obes Metab. 2022; 24(8):1553-1564). In contrast, switching from semaglutide induction to CRB-913 maintenance therapy led to an additional 3.5% weight loss compared with continuous semaglutide treatment at day 41 (p<0.01). DEXA body composition analysis revealed that this effect was driven primarily by a 3.6 g greater reduction in body fat levels, which is consistent with the well-established effects of CB1 inverse agonism on increasing energy expenditure and adipocyte fat metabolism (Matias I, Di Marzo V. Endocannabinoids and the control of energy balance. Trends Endocrinol Metab. 2007; 18 (1): 27-37; Ruiz-Pino F, Muñoz E, Ponce-Díaz F J, Nath Rajeev S, Twitty C, Morales S A. 1716-P: Nimacimab, a peripherally restricted CB1 inhibitor, promotes metabolic homeostasis in a diet-induced obesity (DIO) mouse model as demonstrated by weight loss, restored hormonal regulation, and reduced inflammatory biomarkers. Diabetes. 2025; 74 (Supplement_1): 1716-P). By the end of study, continuous CRB-913 treatment resulted in a greater reduction in fat mass than continuous semaglutide treatment, which accounted for apparent differences in weight loss between the CRB-913/CRB-913 and SEMA/SEMA groups at day 41. Furthermore, semaglutide induction followed by CRB-913 maintenance therapy resulted in reductions in inguinal and epididymal white adipose tissue fat stores that were otherwise refractory to continuous semaglutide monotherapy. This regimen also produced greater improvements in liver pathology than either agent alone, further supporting the potential of CRB-913 to maintain and extend the metabolic benefits induced by incretin-based therapies. These findings highlight the potential advantages of sequential therapy using treatments with complementary mechanisms of weight loss.

The preclinical observations reported here suggest that transitioning from incretin analog induction to CRB-913 maintenance therapy may represent a viable approach for sustaining, and potentially enhancing, the weight loss achieved with incretin analogs. CRB-913 is also orally administered, which may offer a practical advantage over injectable incretin analogs. Targeting the CB1 pathway offers an alternative mechanism for weight loss, which may offer improvements in body composition compared with other approaches, as suggested by the changes in fat stores observed in this study. Notably, other CB1 inverse agonists have also been reported to preserve lean mass while promoting weight loss (Knop F K, Kunos G, Dicker D, et al. Efficacy and safety of monlunabant in adults with obesity and metabolic syndrome: a double-blind, randomized, placebo-controlled, phase 2a trial. Lancet Diabetes Endocrinol. 2025; 13 (11): 911-923; Ruiz-Pino F, Muñoz E, Ponce-Díaz FJ, Nath Rajeev S, Twitty C, Morales S A. 1716-P: Nimacimab, a peripherally restricted CB1 inhibitor, promotes metabolic homeostasis in a diet-induced obesity (DIO) mouse model as demonstrated by weight loss, restored hormonal regulation, and reduced inflammatory biomarkers. Diabetes. 2025; 74 (Supplement_1): 1716-P; Food U, Committee DAA. FDA briefing document: Zimulti (rimonabant) Tablets, 20 mg. Rockville: FSA. 2007).

The findings herein introduce a new potential paradigm for obesity management, in which CRB-913 serves as a maintenance therapy following incretin analog-induced weight loss. CB1 inverse agonism may also overcome resistance to incretin analog therapies and potentially address tolerability issues encountered with their use.

### OTHER EMBODIMENTS

While the present disclosure has been described in connection with specific embodiments thereof, it will be understood that it is capable of further modifications and this application is intended to cover any variations, uses, or adaptations of the present disclosure following, in general, the principles of the present disclosure and including such departures from the present disclosure come within known or customary practice within the art to which the present disclosure pertains and may be applied to the essential features hereinbefore set forth.

1. A solid dispersion composition comprising:
at least one polymeric component; and
substantially amorphous CRB-913:

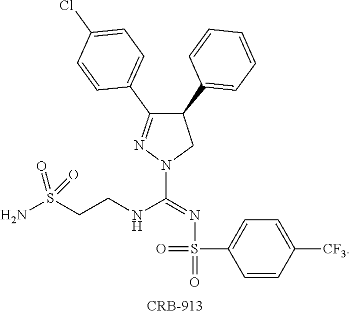 [SMILES: NS(=O)(=O)CCN/C(=N\S(=O)(=O)c1ccc(C(F)(F)F)cc1)N1C[C@H](c2ccccc2)C(c2ccc(Cl)cc2)=N1]

2-50. (canceled)

51. A method of treating a weight condition or weight-related condition in a subject in need thereof, the method comprising orally administering to the subject a total daily dose of 9±5 mg to 600±5 mg of CRB-913:

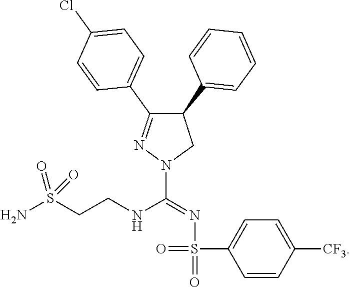 [SMILES: NS(=O)(=O)CCN/C(=N\S(=O)(=O)c1ccc(C(F)(F)F)cc1)N1C[C@H](c2ccccc2)C(c2ccc(Cl)cc2)=N1]

52. A method of maintaining a BMI in a subject previously treated for a weight condition or weight-related condition, the method comprising orally administering to the subject a total daily dose of 9±5 mg to 600±5 mg of CRB-913:

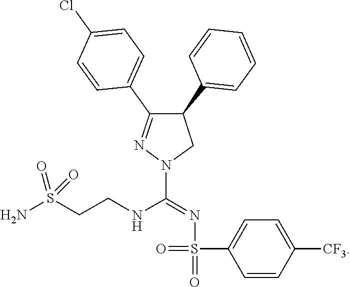 [SMILES: NS(=O)(=O)CCN/C(=N\S(=O)(=O)c1ccc(C(F)(F)F)cc1)N1C[C@H](c2ccccc2)C(c2ccc(Cl)cc2)=N1]

53. The method of claim 51, wherein the subject previously received a weight loss therapy selected from: GLP-1 receptor agonist, a CB1 inverse agonist, a glucose-dependent insulinotropic polypeptide (GIP) modulator, a dual glucose-dependent insulinotropic polypeptide (GIP) modulator and glucagon-like peptide 1 receptor agonist, a triple agonist for weight loss, an amylin receptor agonist, an appetite suppressant, a caloric restriction regimen, gastric bypass, endoscopic sleeve, intragastric balloon, or a fat-absorption inhibitor.

54. The method of claim 51, wherein the weight condition or weight-related condition is obesity, overweight, weight gain, binge eating disorder, bulimia, antipsychotic-induced weight gain, anxiety and depression in schizophrenia, diabetic nephropathy, gastrointestinal conditions, substance use disorders, blood sugar/lipids level imbalances, sleep apnea, cardiovascular disease, Alzheimer's, high cholesterol, or gastrointestinal issues.

55-56. (canceled)

57. The method of claim 51, wherein the method further comprises treating a co-morbidity of obesity, wherein the co-morbidity of obesity is diabetes, dyslipidemia, Metabolic Syndrome, dementia, a cardiovascular disease, a hepatic disease; hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; or increased incidence of malignancy of the ovaries, cervix, uterus, breasts, prostrate, or gallbladder.

58-60. (canceled)

61. The method of claim 51, wherein the subject suffers from type 1 or type 2 diabetes.

62-66. (canceled)

67. The method of claim 1, wherein the total daily dose administered is 9±2 mg to 150±2 mg.

68. The method of claim 1 any one of claims 51 to 66, wherein the total daily dose administered is 9±2 mg to 300±2 mg.

69. (canceled)

70. The method of claim 51, wherein the method comprises administering a total daily dose of 20±2 mg to 80±2 mg of CRB-913.

71. The method of claim 51, wherein the method comprises administering a total daily dose of 70±2 mg to 150±2 mg of CRB-913.

72-87. (canceled)

88. The method of claim 51, wherein the method comprises administering a dose of CRB-913 once a day.

89. The method of claim 88, wherein the daily dose comprises either 20±2 mg, 40±2 mg, 60±2 mg, 75±2 mg, 80±2 mg, 100±2 mg, 150±2 mg, 200±2 mg, 300±2 mg, 400±2 mg, or 600±2 mg of CRB-913.

90. The method of claim 51, wherein the method comprises administering a dose of CRB-913 twice a day or three times a day.

91. (canceled)

92. The method of claim 90, wherein each dose individually comprises either 20±2 mg, 40±2 mg, 60±2 mg, 75±2 mg, 80±2 mg, 100±2 mg, or 150±2 mg of CRB-913.

93. The method of claim 51, wherein CRB-913 is chronically administered to the subject.

94. (canceled)

95. The method of claim 51, wherein the administration comprises:
(i) an initial administration of an average daily dose over a period of 1 to 24 weeks;
(ii) after (i), administering 1.5 to 4 times the average daily dose over a period of 1 to 24 weeks;
(iii) after (ii), administering 1.5 to 4 times the dose previously administered over a period of 1 to 24 weeks; and
(iv) repeating (iii) until at least the subject is a desired weight.

96. (canceled)

97. The method of claim 51, wherein the administering comprises:
(i) an initial administration of an average daily dose over a first period of at least 7 days; and
(ii) following step (i), administration of 1.5 to 4 times the average daily dose over a second period of at least 7 days.

98. (canceled)

99. The method of claim 97, wherein:
step (i) comprises administering to the subject during the first period an average daily dose of from 10±5 mg to 60±5 mg of CRB-913; and
step (ii) comprises administering to the subject during the second period an average daily dose of from 20±5 mg to 200±5 mg of CRB-913.

100-115. (canceled)

116. The method of claim 51, wherein the method further comprises administering to the subject a second therapeutic agent.

117-137. (canceled)
